(19) United States
(12) Patent Application Publication
(10) Pub. No.: US 2017/0173008 A1
(43) Pub. Date: Jun. 22, 2017

(54) ANTIMICROBIAL AGENTS AND SCREENING METHODS

(71) Applicant: PRESIDENT AND FELLOWS OF HARVARD COLLEGE, Cambridge, MA (US)

(72) Inventors: Beckwith Roger JONATHAN, Cambridge, MA (US); Rachel DUTTON, Cambridge, MA (US); Markus ESER, Boston, MA (US); Cristina LANDETA, Boston, MA (US); Jessica L. BLAZYK, Boston, MA (US); Brian M. MEEHAN, Boston, MA (US); Feras HATAHET, Boston, MA (US); Dana BOYD, Medford, MA (US)

(73) Assignee: PRESIDENT AND FELLOWS OF HARVARD COLLEGE, Cambridge, MA (US)

(21) Appl. No.: 15/127,173

(22) PCT Filed: Mar. 19, 2015
(86) PCT No.: PCT/US15/21480

§ 371 Date: Sep. 19, 2016

Related U.S. Application Data
(60) Provisional application No. 61/955,428, filed on Mar. 19, 2014.

(51) Int. Cl. A61K 31/50; A61K 31/501

(52) U.S. Cl. CPC A61K 31/50; A61K 31/501

(57) ABSTRACT

Disclosed herein are antibacterial and antimicrobial compositions and methods of use. Also disclosed are screening assays for identification of an agent that specifically inhibits DsbB or bVKOR. Such methods are useful, for example, in identifying antibacterial and antimicrobial agents and compositions.

### RELATED APPLICATIONS

This Application claims the benefit under 35 U.S.C. §119(e) of U.S. Provisional Application Ser. No. 61/955,428, filed Mar. 19, 2014, the contents of which are incorporated herein by reference in their entirety.

### GOVERNMENTAL SUPPORT

This invention was made with Government support under GMO 41883, NIH U54 AI057159, NIH 5-UL1RR025680-1, and T32-HL07917, each awarded by the National Institutes of Health. The Government has certain rights in the invention.

### FIELD OF THE INVENTION

The present invention relates to the field of antimicrobial agents, particularly antibiotics and screening methods of identification of such agents.

### BACKGROUND OF THE INVENTION

The introduction of disulfide bonds between pairs of cysteines in proteins is an important contributor to the folding and stability of many proteins. In bacteria, this advantageous modification of proteins is generally limited to proteins or domains of proteins that are exported through the cytoplasmic membrane to the cell envelope or beyond. Disulfide bonds are may be critical for the stability of many proteins involved in bacterial virulence. Virulence proteins containing disulfide bonds include toxins, adhesins and those involved in the assembly of flagella, fimbriae, pili, and type II and III secretion systems. Thus, inhibition or inactivation of the enzymes involved in making protein disulfide bonds interfere simultaneously with the folding and activity of multiple virulence factors of these bacterial pathogens. Compounds that inactivate one of these enzymes could have profound effects on the virulence of many bacterial pathogens and thus represent a new class of antibiotics.

It is over twenty years since the enzymes required for protein disulfide bond formation were first discovered in bacteria. Work in a number of laboratories has elaborated on the details of this pathway in E. coli and many other gram-negative bacteria and has provided a host of tools for their study. In these bacteria, disulfide bonds are introduced into substrate proteins as they cross through the cytoplasmic membrane into the cell envelope. The periplasmic enzyme DsbA, a member of the thioredoxin family, oxidizes pairs of cysteines to the disulfide-bonded state through its Cys-X-X-Cys active site motif. The reduced form of DsbA resulting from this reaction is re-oxidized by the membrane protein DsbB, thus regenerating DsbA's activity. DsbB itself is reoxidized by membrane-imbedded quinones, leading to transfer of electrons, under aerobic conditions, to terminal oxidases and oxygen.

While the DsbB/DsbA system is widespread in many classes of the proteo-bacteria, members of certain other classes of bacteria, e.g. the Actinobacteria and Cyanobacteria, use a somewhat different pathway for disulfide bond formation. This alternate pathway retains a DsbA-like protein as the donor of disulfide bonds to substrate proteins, but uses a different membrane protein, VKOR, instead of DsbB to oxidize DsbA. Bacterial VKOR is a homologue of the vertebrate protein, vitamin K epoxide reductase, an endoplasmic reticular enzyme involved in early steps of the blood coagulation pathway and the target of the blood thinner warfarin (Coumadin©). Bacterial VKORs show no homology to DsbB, yet many of their features resemble those of DsbB. Like DsbB, these VKORs have two extra-cytoplasmic soluble domains, each containing a pair of cysteines that are required for the enzyme's function and one of which is a Cys-X-X-Cys motif (not in a thioredoxin-domain). The particular cysteines of DsbA and VKOR that interact are analogous to the ones that interact between DsbA and DsbB. A vkor gene cloned from the Actinobacteria Mycobacterium tuberculosis and expressed in E. coli complements a dsbB null mutant and regenerates oxidized E. coli DsbA.

The main obvious difference between DsbB and VKOR, other than lack of homology, is that the Cys-X-X-Cys motif of the former is in the N-terminal extra-cytoplasmic domain and that of the latter is in the following more C-terminal extra-cytoplasmic domain. Another difference between the two proteins is that at least some bacterial VKORs, like their eukaryotic homologue, are sensitive to the anti-coagulant warfarin (Coumadin©) and other inhibitors of vertebrate VKOR, while DsbB is not. Finally, VKOR is essential for the growth of M. tuberculosis (and M. smegmatis as well) while neither DsbB nor DsbA is essential for the aerobic growth of E. coli.

The spread of multiple drug resistant microbial pathogens (e.g., M. tuberculosis) is an enormous public health problem. The development of antimicrobial agents that have unique targets within the pathogens is needed to facilitate treatment of the multiple drug resistant diseases. The existence of two different bacterial pathways for maintaining the protein DsbA in its oxidized active state, one from certain gram-negative pathogens using DsbB and one from the gram-positive pathogen M. tuberculosis using VKOR, both of which can function in E. coli, suggested a methodology for seeking potential lead compounds for antibiotics active against both classes of bacteria.

### SUMMARY OF THE INVENTION

One aspect of the invention relates to a novel composition (e.g., pharmaceutical and/or antibacterial) comprising a compound of Formula I:

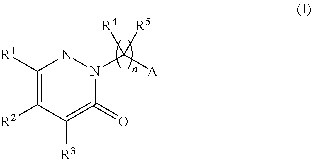

wherein

R¹, R² and R³ are independently selected from the group consisting of hydrogen, deuterium, halogen, cyano, optionally substituted alkyl, optionally substituted cyclyl, optionally substituted heterocyclyl, optionally substituted aryl, optionally substituted heteroaryl, OR⁶, CO₂R⁶, C(O)NR⁶R⁷, OC(O)R⁶, N(R⁶)C(O)R⁶, NR⁶R⁷, SR⁶, S(O)—R⁶, SO₂R⁶, OS(O)₂R⁶, SO₂NR⁶NR⁷, and NO₂;

R⁴ and R⁵ are independently hydrogen, deuterium, optionally substituted alkyl, or halogen, or R⁴ and R⁵ together with the carbon they are attached to form an optionally substituted cyclic alkyl or optionally substituted heterocyclic;

R⁶ and R⁷ are independently for each occurrence hydrogen, optionally substituted alkyl, optionally substituted cyclyl, optionally substituted heterocyclyl, optionally substituted aryl, or optionally substituted heteroaryl; A is aryl, heteroaryl, cyclyl, heterocyclyl, or alkyl, each of which can be optionally substituted; and

n is 0, 1, or 2.

In some embodiments, a compound of Formula I is of Formula II:

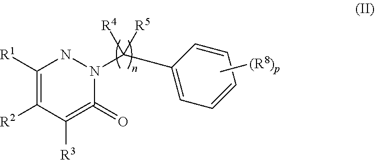

wherein variables are as defined above.

In some embodiments, a compound of Formula II is of Formula II′:

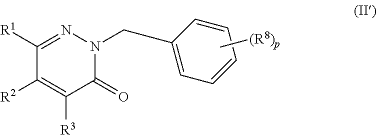

wherein variables are as defined above.

In some other embodiments, a compound of formula II is of formula II″:

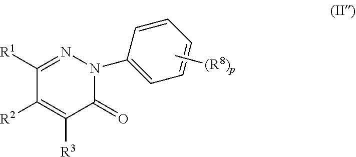

In some embodiments, a compound of Formula I is of Formula III:

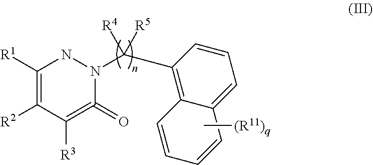

wherein variables are as defined above.

In some embodiments a compound of formula III is of formula III′:

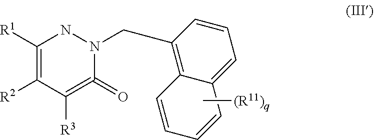

wherein variables are as defined above.

In one embodiment the compound of Formula I, II or III is selected from Table 1.

In one embodiment, the composition (e.g., pharmaceutical and/or antibacterial) comprises a compound of Formula (I), wherein R¹ is H, R² is Cl or Br, R³ is Cl, Br, methyl, methoxy, ethoxy, pyrrolidinyl or butylamino, n is 0 or 1, and A is phenyl, 2-chlorophenyl, 2-bromophenyl, 2-fluorophenyl, 2-methylphenyl, 2-trifluoromethylphenyl, 2-trifluoromethoxyphenyl, 2-cyanophenyl, 2-nitrophenyl, thiophene, 3-chlorothiophene, pyridine or 3-chloropyridine.

In one embodiment, the composition (e.g., pharmaceutical and/or antibacterial) comprises a compound of Formula (I), wherein R¹ is H, R² is Cl or Br, R³ is Cl or Br, n is 0 or 1, and A is phenyl, 2-chlorophenyl, 2-bromophenyl, 2-fluorophenyl, 2-methylphenyl, 2-trifluoromethylphenyl, 2-trifluoromethoxyphenyl, 2-cyanophenyl, 2-nitrophenyl, thiophene, 3-chlorothiophene, pyridine or 3-chloropyridine.

In one embodiment, the compound inhibits DsbB of one or more bacteria, and has an IC50 determined with an in vitro E. coli assay with strain DHB7935 of ≦50 μM, ≦25 μM, ≦12 μM, ≦9 μM, ≦8 μM, ≦6 μM, ≦3 μM, ≦2 μM, ≦1 μM, ≦0.5 μM, ≦0.4 μM, ≦0.3 μM, δ0.2 μM, ≦0.1 μM, ≦0.09 μM, ≦0.08 μM, ≦0.07 μM, ≦0.06 μM, ≦0.05 μM, ≦0.04 μM, ≦0.03 μM, ≦0.02 μM, or ≦0.01 μM.

In one embodiment, the antibacterial composition described above further comprises an agent selected from the group consisting of an antibiotic, an antiseptic, and an antifouling agent.

Another aspect of the invention relates to a matrix impregnated with any of the compositions described above. In one embodiment, the matrix is a gel coating specifically formulated for slow release of the antibacterial composition into a surrounding aqueous environment.

Another aspect of the invention relates to a method comprising administering a therapeutically effective amount of a pharmaceutical composition described herein to a subject with a bacterial infection.

Another aspect of the invention relates to a method of inhibiting a bacteria (e.g., growth of a bacteria) in a subject comprising administering a therapeutically effective amount of a pharmaceutical composition described herein to the subject.

Another aspect of the invention relates to a method of inhibiting a bacteria (e.g., growth of a bacteria) comprising contacting the bacteria with an effective amount of the antibacterial composition described herein.

Another aspect of the invention relates to a method of sensitizing a bacteria to growth inhibition comprising contacting the bacteria with an effective amount of the composition described herein.

Another aspect of the invention relates to a method of inhibiting the development of resistance to an antibiotic by a bacteria comprising, contacting the bacteria with an effective amount of a composition described herein and with an effective amount of the antibiotic.

In one embodiment of the methods herein described, the bacteria is contacted with the compound Formula I of the composition at a concentration of from about 0.25 μM to about 500 μM.

In one embodiment of the methods herein described, the bacterial is a gram (−) bacteria.

In one embodiment of the methods herein described, the bacteria is a pathogen.

In one embodiment of the methods herein described, the bacteria is selected from the group consisting of Salmonella typhimurium, Klebsiella pneumoniae, Vibrio cholera, Haemophilus influenza, Francisella tularensis, Klebsiella oxytoca, Enterobacter cloacae, Enterobacter aerogenes, Citrobacter freundii, Pseudomonas aeruginosa, Acinetobacter baumannii, Helicobacter pylori, and combinations thereof.

Another aspect of the invention relates to a method for identifying an agent that specifically inhibits DsbB. The method comprises testing one or more test agents in a β-gal disulfide bond formation assay using β-gal fused to a bacterial membrane protein, wherein DsbB functions as the oxidant of DsbA in the assay, and identifying test agents that significantly inhibit disulfide bond formation in the assay, and further testing the identified test agent(s) in a β-gal disulfide bond formation assay using β-gal fused to a bacterial membrane protein, wherein bVKOR functions as the oxidant of DsbA in the assay. The ability of the test agent(s) to significantly inhibit disulfide bond formation in the first assay and the inability of the test agent(s) to inhibit disulfide bond formation in the second assay indicates that the test agent(s) specifically inhibits DsbB.

Another aspect of the invention relates to a method for identifying an agent that specifically inhibits bVKOR, comprising the steps testing one or more test agents in a β-gal disulfide bond formation assay using β-gal fused to a bacterial membrane protein, wherein bVKOR functions as the oxidant of DsbA in the assay, and identifying test agents that significantly inhibit disulfide bond formation in the assay, and further testing the identified test agent in a β-gal disulfide bond formation assay using β-gal fused to a bacterial membrane protein, wherein DsbB functions as the oxidant of DsbA in the assay, wherein the ability of the test agent to significantly inhibit disulfide bond formation in the first assay and the inability of the test agent to inhibit disulfide bond formation in the second assay indicates that the test agent specifically inhibits bVKOR.

In one embodiment of the various methods described herein, the β-gal disulfide bond formation assay is performed as a color assay with bacteria grown on agar that comprise 5-bromo-4-chloro-3-indolyl-β-D-galactopyranoside (BCIG), and color readout is performed by a non-human machine.

In one embodiment of the various methods described herein, the bVKOR is from M. tuberculosis.

In one embodiment of the various methods described herein, the β-gal disulfide bond formation assay is performed in E. coli.

In one embodiment of the various methods described herein, the bacterial membrane protein is MalF.

In one embodiment of the various compositions and methods described herein, one or more of the compounds specified in Table 1 and/or Table 9 and/or Table 10 is specifically excluded as the compound. For example, in one embodiment of the various compositions and methods described herein, the compound is not 16.27. In one embodiment of the various compositions and methods described herein, one or more of the following molecules listed in Table 1 and/or Table 9 (1, 4, 8, 23, 18, 16.6, 16.12, 16.20, 16.2, 16.23, 16.13, 16, 16.14, 16.17, 16.24, 16.4, 16.22, 14, 15, 13, 16.8, 12, 17, 16, 16, 16.11, 16.9, 16.7, 16.21, 16.1, 16.3, 16.5, 16.10, 16.15, 16.18, 16.19, 16.25, 16.26, 16.28, 16.29, 16.30, 16.31, 16.32, 16.33, 16.34, 16.35, 16.36, 16.37, 16.38, 16.39, 16.40, 16.41, 16.42, 16.43, or 16.44) is specifically excluded as the compound.

In one embodiment of the various compositions and methods described herein, the compound is 16.25, 16.26, 16.27, 16.28, 16.29, 16.30, 16.31, 16.32, 16.33, 16.34, 16.35, 16.36, 16.37, 16.38, 16.39, 16.40, 16.41, 16.42, 16.43, or 16.44. In one embodiment of the various compositions and methods described herein, the compound is 16.25, 16.26, 16.28, 16.29, 16.30, 16.31, 16.32, 16.33, 16.34, 16.35, 16.36, 16.37, 16.38, 16.39, 16.40, 16.41, 16.42, 16.43, or 16.44.

### Definitions

As the term is used herein, “bacterial VKOR” or “bVKOR” refers to the bacterial homolog of human VKOR that is identified as contained in a variety of microbes (Dutton et al., PNAS 105: 11933-11938 (2008)), such as the microbes identified herein. One example of bacterial VKOR is Mycobacterial tuberculosis VKOR

The compounds referred to in the various tables herein are referred to by a specific assigned number. That number may include a “C” at the beginning, or on occasion the “C” is not present. The assigned numbers with or without the letter “C” designation are intended to refer to the same compound.

As used herein, the term “inhibitor of bacteria”, or “bacterial inhibitor”, or “inhibitor” when used in the context of affecting a bacteria, without further reference, broadly encompasses the inhibition of growth and/or the inhibition of virulence.

As used herein, the term “potentiate” or “potentiator” refers to the activity of the compositions identified herein to potentiate the activity of an agent for inhibiting (e.g., growth or virulence) of a bacteria. Without being bound by theory, it is thought that the potentiating affect arises from the inhibitor activity of the compound, and may also further arise from the activity of the compound to increase access/transport of such agent into the bacteria (e.g. by increasing the porosity of the bacterial outer membrane).

“Beneficial results” may include, but are in no way limited to, lessening or alleviating the severity of the disease condition, preventing the disease condition from worsening, curing the disease condition and prolonging a patient's life or life expectancy. The disease conditions may relate to or may be modulated by the central nervous system.

“Mammal” as used herein refers to any member of the class Mammalia, including, without limitation, humans and nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult and newborn subjects, as well as fetuses, whether male or female, are intended to be included within the scope of this term.

“Small molecule” as used herein refers to an organic compound that may serve to regulate a biological process of the present invention and whose molecular weight limit is approximately 600 Dalton, and may be 900 Dalton or more, allowing for the possibility to rapidly diffuse across cell membranes so that they can reach intracellular sites of action.

“Therapeutic agent” as used herein refers to any substance used internally or externally as a medicine for the treatment, cure, prevention, slowing down, or lessening of a disease or disorder, even if the treatment, cure, prevention, slowing down, or lessening of the disease or disorder is ultimately unsuccessful.

“Therapeutically effective amount” as used herein refers to an amount which is capable of achieving beneficial results in a patient with a condition or a disease condition in which treatment is sought. A therapeutically effective amount can be determined on an individual basis and will be based, at least in part, on consideration of the physiological characteristics of the mammal, the type of delivery system or therapeutic technique used and the time of administration relative to the progression of the disease.

“Treatment” and “treating,” as used herein refer to both therapeutic treatment and prophylactic or preventative measures, wherein the object is to prevent, slow down and/or alleviate the disease or disease condition even if the treatment is ultimately unsuccessful.

The term “derivative” as used herein refers to a chemical substance related structurally to another, i.e., an “original” substance, which can be referred to as a “parent” compound. A “derivative” can be made from the structurally-related parent compound in one or more steps. In one embodiment, the general physical and chemical properties of a derivative can be similar to or different from the parent compound.

The term “subject” and “individual” are used interchangeably herein, and refer to an animal, for example a human, to whom treatment, including prophylactic treatment, with a composition as described herein, is provided. The term “mammal” is intended to encompass a singular “mammal” and plural “mammals,” and includes, but is not limited: to humans, primates such as apes, monkeys, orangutans, and chimpanzees; canids such as dogs and wolves; felids such as cats, lions, and tigers; equids such as horses, donkeys, and zebras, food animals such as cows, pigs, and sheep; ungulates such as deer and giraffes; rodents such as mice, rats, hamsters and guinea pigs; and bears. Preferably, the mammal is a human subject. As used herein, a “subject” refers to a mammal, preferably a human. The term “individual”, “subject”, and “patient” are used interchangeably.

The phrases “parenteral administration” and “administered parenterally” as used herein means modes of administration other than enteral and topical administration, usually by injection, and includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion.

The term “administration” as used herein refers to the presentation of formulations of pharmaceutical compositions described herein, to a subject in a therapeutically effective amount, and includes all routes for dosing or administering drugs or other therapeutics, whether self-administered or administered by medical practitioners. Generally an agent of the present invention is to be administered in the form of a pharmaceutical composition. Pharmaceutical compositions are considered pharmaceutically acceptable for administration to a living organism. For example, they are sterile, the appropriate pH, and ionic strength, for administration. They generally contain the agent formulated in a composition within/in combination with a pharmaceutically acceptable carrier, also known in the art as excipients.

The “pharmaceutically acceptable carrier” means any pharmaceutically acceptable means to mix and/or deliver the targeted delivery composition to a subject. The term “pharmaceutically acceptable carrier” as used herein means a pharmaceutically acceptable material, composition or vehicle, such as a liquid or solid filler, diluent, excipient, solvent or encapsulating material, involved in carrying or transporting the subject agents from one organ, or portion of the body, to another organ, or portion of the body. Each carrier must be “acceptable” in the sense of being compatible with the other ingredients of the formulation and is compatible with administration to a subject, for example a human.

“Inhibit”, as the term is used herein in reference to a bacteria (e.g., to inhibit a bacteria), refers to either partial or complete inhibition of activity, growth, or virulence, or any combination thereof, and is expected to be a reproducibly detectable, statistically significant amount of inhibition, as determined by means known in the art. This activity may be specific for one or more bacteria, examples of which are described herein.

An “indwelling device” is a device that is invasive, placed in or planted within the body, and is associated with a risk of infection.

“Coating agents” are formulations whereby when applied to a substrate surface, a layer or residue of an effective amount of the compound is left deposited on that surface, to thereby inhibit bacteria and/or potentiate a second agent. Examples of coating agents include, without limitation, paints, stains, sealants, waxes, and cleaning products such as disinfectants. In one embodiment, the coating agent is a polymer.

A “substrate surface”, as the term is used herein, refers to the specific surface on which the compound is to be delivered (e.g., via a coating agent). The surface is either external or internal, and is exposed to an aqueous environment which may contain bacteria.

As used herein, the term “test agent” is used to refer to an agent that is to be tested for a specified activity. Once identified as having that activity, it can then be referred to as an agent with that specified activity.

As used herein, a “test agent” or “agent” can be any purified molecule, substantially purified molecule, molecules that are one or more components of a mixture of compounds, or a mixture of a compound with any other material that can be analyzed using the methods of the present invention.

As used herein, the term “aliphatic” means a moiety characterized by a straight or branched chain arrangement of constituent carbon atoms and can be saturated or partially unsaturated with one or more (e.g., one, two, three, four, five or more) double or triple bonds.

As used herein, the term “alicyclic” means a moiety comprising a nonaromatic ring structure. Alicyclic moieties can be saturated or partially unsaturated with one or more double or triple bonds. Alicyclic moieties can also optionally comprise heteroatoms such as nitrogen, oxygen and sulfur. The nitrogen atoms can be optionally quaternerized or oxidized and the sulfur atoms can be optionally oxidized. Examples of alicyclic moieties include, but are not limited to moieties with C₃-C₈ rings such as cyclopropyl, cyclohexane, cyclopentane, cyclopentene, cyclopentadiene, cyclohexane, cyclohexene, cyclohexadiene, cycloheptane, cycloheptene, cycloheptadiene, cyclooctane, cyclooctene, and cyclooctadiene.

As used herein, the term “alkyl” means a straight or branched, saturated aliphatic radical having a chain of carbon atoms. Cₓ alkyl and Cₓ-C_{y}alkyl are typically used where X and Y indicate the number of carbon atoms in the chain. For example, C₁-C₆alkyl includes alkyls that have a chain of between 1 and 6 carbons (e.g., methyl, ethyl, propyl, isopropyl, butyl, sec-butyl, isobutyl, tert-butyl, pentyl, neopentyl, hexyl, and the like). Alkyl represented along with another radical (e.g., as in arylalkyl) means a straight or branched, saturated alkyl divalent radical having the number of atoms indicated or when no atoms are indicated means a bond, e.g., (C₆-C₁₀)aryl(C₀-C₃)alkyl includes phenyl, benzyl, phenethyl, 1-phenylethyl 3-phenylpropyl, and the like. Backbone of the alkyl can be optionally inserted with one or more heteroatoms, such as N, O, or S.

In preferred embodiments, a straight chain or branched chain alkyl has 30 or fewer carbon atoms in its backbone (e.g., C1-C30 for straight chains, C3-C30 for branched chains), and more preferably 20 or fewer. Likewise, preferred cycloalkyls have from 3-10 carbon atoms in their ring structure, and more preferably have 5, 6 or 7 carbons in the ring structure. The term “alkyl” (or “lower alkyl”) as used throughout the specification, examples, and claims is intended to include both “unsubstituted alkyls” and “substituted alkyls”, the latter of which refers to alkyl moieties having one or more substituents replacing a hydrogen on one or more carbons of the hydrocarbon backbone.

Unless the number of carbons is otherwise specified, “lower alkyl” as used herein means an alkyl group, as defined above, but having from one to ten carbons, more preferably from one to six carbon atoms in its backbone structure. Likewise, “lower alkenyl” and “lower alkynyl” have similar chain lengths. Throughout the application, preferred alkyl groups are lower alkyls. In preferred embodiments, a substituent designated herein as alkyl is a lower alkyl.

In one embodiment, alkyl is C1-12alkyl. In one embodiment, alkyl is C1-8alkyl. In one embodiment, alkyl is C1-6alkyl. In one embodiment, alkyl is C1-4alkyl. In one embodiment, alkyl is methyl, ethyl, n-propyl, i-propyl, n-butyl, i-butyl, or t-butyl.

Substituents of a substituted alkyl can include halogen, hydroxy, nitro, thiols, amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF₃, —CN and the like.

As used herein, the term alkyl includes alkenyl and alkynyl. The term “alkenyl” refers to unsaturated straight-chain, branched-chain or cyclic hydrocarbon radicals having at least one carbon-carbon double bond. Cₓ alkenyl and Cₓ-C_{y}alkenyl are typically used where X and Y indicate the number of carbon atoms in the chain. For example, C₂-C₆alkenyl includes alkenyls that have a chain of between 1 and 6 carbons and at least one double bond, e.g., vinyl, allyl, propenyl, isopropenyl, 1-butenyl, 2-butenyl, 3-butenyl, 2-methylallyl, 1-hexenyl, 2-hexenyl, 3-hexenyl, and the like). Alkenyl represented along with another radical (e.g., as in arylalkenyl) means a straight or branched, alkenyl divalent radical having the number of atoms indicated. Backbone of the alkenyl can be optionally inserted with one or more heteroatoms, such as N, O, or S.

As used herein, the term “alkynyl” refers to unsaturated hydrocarbon radicals having at least one carbon-carbon triple bond. Cₓ alkynyl and Cₓ-C_{y}alkynyl are typically used where X and Y indicate the number of carbon atoms in the chain. For example, C₂-C₆alkynyl includes alkynls that have a chain of between 1 and 6 carbons and at least one triple bond, e.g., ethynyl, 1-propynyl, 2-propynyl, 1-butynyl, isopentynyl, 1,3-hexa-diyn-yl, n-hexynyl, 3-pentynyl, 1-hexen-3-ynyl and the like. Alkynyl represented along with another radical (e.g., as in arylalkynyl) means a straight or branched, alkynyl divalent radical having the number of atoms indicated. Backbone of the alkynyl can be optionally inserted with one or more heteroatoms, such as N, O, or S.

The term “heteroalkyl”, as used herein, refers to straight or branched chain, or cyclic carbon-containing radicals, or combinations thereof containing at least one heteroatom. Suitable heteroatoms include, but are not limited to, O, N, Si, P, Se, B, and S, wherein the phosphorous and sulfur atoms are optionally oxidized, and the nitrogen heteroatom is optionally quaternized. Heteroalkyls can be substituted as defined above for alkyl groups.

As used herein, the term “halogen” or “halo” refers to an atom selected from fluorine, chlorine, bromine and iodine. The term “halogen radioisotope” or “halo isotope” refers to a radionuclide of an atom selected from fluorine, chlorine, bromine and iodine.

A “halogen-substituted moiety” or “halo-substituted moiety”, as an isolated group or part of a larger group, means an aliphatic, alicyclic, or aromatic moiety, as described herein, substituted by one or more “halo” atoms, as such terms are defined in this application. For example, halo-substituted alkyl includes haloalkyl, dihaloalkyl, trihaloalkyl, perhaloalkyl and the like (e.g. halosubstituted (C₁-C₃)alkyl includes chloromethyl, dichloromethyl, difluoromethyl, trifluoromethyl (—CF₃), 2,2,2-trifluoroethyl, perfluoroethyl, 2,2,2-trifluoro-1,1-dichloroethyl, and the like).

The term “aryl” refers to monocyclic, bicyclic, or tricyclic fused aromatic ring system Cₓ aryl and Cₓ-C_{y}aryl are typically used where X and Y indicate the number of carbon atoms in the ring system Exemplary aryl groups include, but are not limited to, pyridinyl, pyrimidinyl, furanyl, thienyl, imidazolyl, thiazolyl, pyrazolyl, pyridazinyl, pyrazinyl, triazinyl, tetrazolyl, indolyl, benzyl, phenyl, naphthyl, anthracenyl, azulenyl, fluorenyl, indanyl, indenyl, naphthyl, phenyl, tetrahydronaphthyl, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH carbazolyl, carbolinyl, chromanyl, chromenyl, cinnolinyl, decahydroquinolinyl, 2H, 6H-1,5,2-dithiazinyl, dihydrofuro[2,3 b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl and xanthenyl, and the like. In some embodiments, 1, 2, 3, or 4 hydrogen atoms of each ring can be substituted by a substituent.

The term “heteroaryl” refers to an aromatic 5-8 membered monocyclic, 8-12 membered fused bicyclic, or 11-14 membered fused tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively. Cₓ heteroaryl and Cₓ-C_{y}heteroaryl are typically used where X and Y indicate the number of carbon atoms in the ring system Heteroaryls include, but are not limited to, those derived from benzo[b]furan, benzo[b] thiophene, benzimidazole, imidazo[4,5-c]pyridine, quinazoline, thieno[2,3-c]pyridine, thieno[3,2-b]pyridine, thieno[2, 3-b]pyridine, indolizine, imidazo[1,2a]pyridine, quinoline, isoquinoline, phthalazine, quinoxaline, naphthyridine, quinolizine, indole, isoindole, indazole, indoline, benzoxazole, benzopyrazole, benzothiazole, imidazo[1,5-a]pyridine, pyrazolo[1,5-a]pyridine, imidazo[1,2-a]pyrimidine, imidazo[1,2-c]pyrimidine, imidazo[1,5-a]pyrimidine, imidazo[1,5-c]pyrimidine, pyrrolo[2,3-b]pyridine, pyrrolo[2,3cjpyridine, pyrrolo[3,2-c]pyridine, pyrrolo[3,2-b]pyridine, pyrrolo[2,3-d]pyrimidine, pyrrolo[3,2-d]pyrimidine, pyrrolo [2,3-b]pyrazine, pyrazolo[1,5-a]pyridine, pyrrolo[, 2-b]pyridazine, pyrrolo[1,2-c]pyrimidine, pyrrolo[1,2-a]pyrimidine, pyrrolo[1,2-a]pyrazine, triazo[1,5-a]pyridine, pteridine, purine, carbazole, acridine, phenazine, phenothiazene, phenoxazine, 1,2-dihydropyrrolo[3,2,1-hi]indole, indolizine, pyrido[1,2-a]indole, 2(1H)-pyridinone, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, decahydroquinolinyl, 2H, 6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxepanyl, oxetanyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydropyranyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl and xanthenyl. Some exemplary heteroaryl groups include, but are not limited to, pyridinyl, pyridyl, furyl or furanyl, imidazolyl, benzimidazolyl, pyrimidinyl, thiophenyl or thienyl, pyridazinyl, pyrazinyl, quinolinyl, indolyl, thiazolyl, naphthyridinyl, 2-amino-4-oxo-3,4-dihydropteridin-6-yl, tetrahydroisoquinolinyl, and the like. In some embodiments, 1, 2, 3, or 4 hydrogen atoms of each ring may be substituted by a substituent.

In some embodiments, the heteroaryl can be furan, thiophene, pyrrole, 1,2-oxathiolane, isoxazole, oxazole, or silole. In some embodiments, the heterocyclyl is a 6-membered heterocyclic. In some embodiments, heteroaryl can be pyridine, pyran, oxazine, thiazine, pyrimidine, piperazine, thiine, thiadiazine or dithiazine.

Aryl and heteroaryls can be optionally substituted with one or more substituents selected for example from halogen, alkyl, aralkyl, alkenyl, alkynyl, cycloalkyl, hydroxyl, amino, nitro, sulfhydryl, imino, amido, phosphate, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —OCF₃, —CN, or the like.

The term “cyclyl” or “cycloalkyl” or “cyclic alkyl” refers to saturated and partially unsaturated cyclic hydrocarbon groups having 3 to 12 carbons, for example, 3 to 8 carbons, and, for example, 3 to 6 carbons. Cₓcyclyl and Cₓ-C_{y}cylcyl are typically used where X and Y indicate the number of carbon atoms in the ring system. The cycloalkyl group additionally can be optionally substituted, e.g., with 1, 2, 3, or 4 substituents. C₃-C₁₀cyclyl includes cyclopropyl, cyclobutyl, cyclopentyl, cyclohexyl, cyclohexenyl, 2,5-cyclohexadienyl, cycloheptyl, cyclooctyl, bicyclo[2.2.2]octyl, adamantan-1-yl, decahydronaphthyl, oxocyclohexyl, dioxocyclohexyl, thiocyclohexyl, 2-oxobicyclo [2.2.1]hept-1-yl, and the like.

The term “heterocyclyl” refers to a nonaromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively). Cₓheterocyclyl and Cₓ-C_{y}heterocyclyl are typically used where X and Y indicate the number of carbon atoms in the ring system. In some embodiments, 1, 2 or 3 hydrogen atoms of each ring can be substituted by a substituent. Exemplary heterocyclyl groups include, but are not limited to piperazinyl, pyrrolidinyl, dioxanyl, morpholinyl, tetrahydrofuranyl, piperidyl, 4-morpholyl, 4-piperazinyl, pyrrolidinyl, perhydropyrrolizinyl, 1,4-diazaperhydroepinyl, 1,3-dioxanyl, 1,4-dioxanyland the like.

In some embodiments, the heterocyclyl is a 5-membered heterocyclic. In some other embodiments, the heterocyclyl is a 6-membered heterocyclic.

The terms “bicyclic” and “tricyclic” refers to fused, bridged, or joined by a single bond polycyclic ring assemblies.

The term “cyclylalkylene” means a divalent aryl, heteroaryl, cyclyl, or heterocyclyl.

As used herein, the term “fused ring” refers to a ring that is bonded to another ring to form a compound having a bicyclic structure when the ring atoms that are common to both rings are directly bound to each other. Non-exclusive examples of common fused rings include decalin, naphthalene, anthracene, phenanthrene, indole, furan, benzofuran, quinoline, and the like. Compounds having fused ring systems can be saturated, partially saturated, cyclyl, heterocyclyl, aromatics, heteroaromatics, and the like.

As used herein, the term “carbonyl” means the radical —C(O)—. It is noted that the carbonyl radical can be further substituted with a variety of substituents to form different carbonyl groups including acids, acid halides, amides, esters, ketones, and the like.

The term “carboxy” means the radical —C(O)O—. It is noted that compounds described herein containing carboxy moieties can include protected derivatives thereof, i.e., where the oxygen is substituted with a protecting group. Suitable protecting groups for carboxy moieties include benzyl, tert-butyl, and the like. The term “carboxyl” means —COOH

The term “cyano” means the radical —CN.

The term, “heteroatom” refers to an atom that is not a carbon atom. Particular examples of heteroatoms include, but are not limited to nitrogen, oxygen, sulfur and halogens. A “heteroatom moiety” includes a moiety where the atom by which the moiety is attached is not a carbon. Examples of heteroatom moieties include —N═, —NR^{N}—, —N⁺(O⁻)═, —O—, —S— or —S(O)₂—, —OS(O)₂—, and —SS—, wherein R^{N} is H or a further substituent.

The term “hydroxy” means the radical —OH.

The term “imine derivative” means a derivative comprising the moiety —C(NR)—, wherein R comprises a hydrogen or carbon atom alpha to the nitrogen.

The term “nitro” means the radical —NO₂.

An “oxaaliphatic,” “oxaalicyclic”, or “oxaaromatic” mean an aliphatic, alicyclic, or aromatic, as defined herein, except where one or more oxygen atoms (—O—) are positioned between carbon atoms of the aliphatic, alicyclic, or aromatic respectively.

An “oxoaliphatic,” “oxoalicyclic”, or “oxoaromatic” means an aliphatic, alicyclic, or aromatic, as defined herein, substituted with a carbonyl group. The carbonyl group can be an aldehyde, ketone, ester, amide, acid, or acid halide.

As used herein, the term, “aromatic” means a moiety wherein the constituent atoms make up an unsaturated ring system, all atoms in the ring system are sp² hybridized and the total number of pi electrons is equal to 4n+2. An aromatic ring can be such that the ring atoms are only carbon atoms (e.g., aryl) or can include carbon and non-carbon atoms (e.g., heteroaryl).

As used herein, the term “substituted” refers to independent replacement of one or more (typically 1, 2, 3, 4, or 5) of the hydrogen atoms on the substituted moiety with substituents independently selected from the group of substituents listed below in the definition for “substituents” or otherwise specified. In general, a non-hydrogen substituent can be any substituent that can be bound to an atom of the given moiety that is specified to be substituted. Examples of substituents include, but are not limited to, acyl, acylamino, acyloxy, aldehyde, alicyclic, aliphatic, alkanesulfonamido, alkanesulfonyl, alkaryl, alkenyl, alkoxy, alkoxycarbonyl, alkyl, alkylamino, alkylcarbanoyl, alkylene, alkylidene, alkylthios, alkynyl, amide, amido, amino, amino, aminoalkyl, aralkyl, aralkylsulfonamido, arenesulfonamido, arenesulfonyl, aromatic, aryl, arylamino, arylcarbanoyl, aryloxy, azido, carbamoyl, carbonyl, carbonyls (including ketones, carboxy, carboxylates, CF₃, OCF₃, cyano (CN), cycloalkyl, cycloalkylene, ester, ether, haloalkyl, halogen, halogen, heteroaryl, heterocyclyl, hydroxy, hydroxy, hydroxyalkyl, imino, iminoketone, ketone, mercapto, nitro, oxaalkyl, oxo, oxoalkyl, phosphoryl (including phosphonate and phosphinate), silyl groups, sulfonamido, sulfonyl (including sulfate, sulfamoyl and sulfonate), thiols, and ureido moieties, each of which may optionally also be substituted or unsubstituted. In some cases, two substituents, together with the carbon(s) to which they are attached to, can form a ring.

The terms “alkoxyl” or “alkoxy” as used herein refers to an alkyl group, as defined above, having an oxygen radical attached thereto, i.e. —O-alkyl group. In some embodiments, alkoxy is —O—C1-12alkyl, —O—C1-10alkyl, —O—C1-8alkyl, —O—C1-6alkyl, or —O—C1-4alkyl. Representative alkoxyl groups include methoxy, ethoxy, propyloxy, tert-butoxy, n-propyloxy, iso-propyloxy, n-butyloxy, iso-butyloxy, and the like.

An “ether” is two hydrocarbons covalently linked by an oxygen. Accordingly, the substituent of an alkyl that renders that alkyl an ether is or resembles an alkoxyl, such as can be represented by one of —O-alkyl, —O-alkenyl, and —O-alkynyl. Aroxy can be represented by —O-aryl or O-heteroaryl, wherein aryl and heteroaryl are as defined below. The alkoxy and aroxy groups can be substituted as described above for alkyl.

The term “aralkyl”, as used herein, refers to an alkyl group substituted with an aryl group (e.g., an aromatic or heteroaromatic group).

The term “alkylthio” refers to an alkyl group, as defined above, having a sulfur radical attached thereto. In some embodiments, the “alkylthio” moiety is represented by one of —S-alkyl, —S-alkenyl, and —S-alkynyl. In some embodiments, alkylthio is —S—C1-12alkyl, —S—C1-10alkyl, —S—C1-8alkyl, —S—C1-6alkyl, or —S—C1-4alkyl. Representative alkylthio groups include methylthio, ethylthio, —S-n-propyl, —S-i-propyl, —S-n-butyl, —S-i-butyl, —S-t-butyl, and the like. The term “alkylthio” also encompasses cycloalkyl groups, alkene and cycloalkene groups, and alkyne groups. “Arylthio” refers to aryl or heteroaryl groups.

The term “sulfinyl” means the radical —SO—. It is noted that the sulfinyl radical can be further substituted with a variety of substituents to form different sulfinyl groups including sulfinic acids, sulfinamides, sulfinyl esters, sulfoxides, and the like.

The term “sulfonyl” means the radical —SO₂—. It is noted that the sulfonyl radical can be further substituted with a variety of substituents to form different sulfonyl groups including sulfonic acids (—SO₃H), sulfonamides, sulfonate esters, sulfones, and the like. Exemplary sulfonate groups include mesylate (—OS(O)₂Me), triflate (—OS(O)₂CF₃), besylate (—OS(O)₂Ph) and tosylate (—OS(O)₂C₆H₄CH₃).

The term “thiocarbonyl” means the radical —C(S)—. It is noted that the thiocarbonyl radical can be further substituted with a variety of substituents to form different thiocarbonyl groups including thioacids, thioamides, thioesters, thioketones, and the like.

As used herein, the term “amino” means —NH₂. The term “alkylamino” means a nitrogen moiety having at least one straight or branched unsaturated aliphatic, cyclyl, or heterocyclyl radicals attached to the nitrogen. The term “mono- or di-alkylamino” means —NH(alkyl) or —N(alkyl)(alkyl), respectively. Representative alkylamino groups include —NH(C₁-C₁₀alkyl), —N(C₁-C₁₀alkyl)₂, and the like. In some embodiments, alkylamino is a mono-alkylamino, i.e., —N(H)-alkyl. In some embodiments, mono-alkylamino is —N(H)—C1-12alkyl, —N(H)—C1-10alkyl, —N(H)—C1-8alkyl, —N(H)—C1-6alkyl, or —N(H)—C1-4alkyl. In one embodiment, mono-alkylamino is —N(H)-methyl, —N(H)-ethyl, —N(H)-n-propyl, —N(H)-i-propyl, —N(H)-n-butyl, —N(H)-i-butyl, or —N(H)-t-butyl.

The term “alkylamino” includes “alkenylamino,” “alkynylamino,” “cyclylamino,” and “heterocyclylamino.” The term “arylamino” means a nitrogen moiety having at least one aryl radical attached to the nitrogen. For example —NHaryl, and —N(aryl)₂. The term “heteroarylamino” means a nitrogen moiety having at least one heteroaryl radical attached to the nitrogen. For example —NHheteroaryl, and —N(heteroaryl)₂. Optionally, two substituents together with the nitrogen can also form a ring. Unless indicated otherwise, the compounds described herein containing amino moieties can include protected derivatives thereof. Suitable protecting groups for amino moieties include acetyl, tertbutoxycarbonyl, benzyloxycarbonyl, and the like.

The term “aminoalkyl” means an alkyl, alkenyl, and alkynyl as defined above, except where one or more substituted or unsubstituted nitrogen atoms (—N—) are positioned between carbon atoms of the alkyl, alkenyl, or alkynyl. For example, an (C₂-C₆) aminoalkyl refers to a chain comprising between 2 and 6 carbons and one or more nitrogen atoms positioned between the carbon atoms.

The term “alkoxyalkoxy” means —O-(alkyl)-O-(alkyl), such as —OCH₂CH₂OCH₃, and the like.

The term “alkoxycarbonyl” means —C(O)O-(alkyl), such as —C(═O)OCH₃, —C(═O)OCH₂CH₃, and the like.

The term “alkoxyalkyl” means -(alkyl)-O-(alkyl), such as —CH₂OCH₃, —CH₂OCH₂CH₃, and the like.

The term “aryloxy” means —O-(aryl), such as —O-phenyl, —O-pyridinyl, and the like.

The term “arylalkyl” means -(alkyl)-(aryl), such as benzyl (i.e., —CH₂phenyl), —CH₂— pyrindinyl, and the like.

The term “arylalkyloxy” means —O-(alkyl)-(aryl), such as —O-benzyl, —O—CH₂-pyridinyl, and the like.

The term “cycloalkyloxy” means —O-(cycloalkyl), such as —O-cyclohexyl, and the like.

The term “cycloalkylalkyloxy” means —O-(alkyl)-(cycloalkyl, such as —OCH₂cyclohexyl, and the like.

The term “aminoalkoxy” means —O-(alkyl)-NH₂, such as —OCH₂NH₂, —OCH₂CH₂NH₂, and the like.

The term “mono- or di-alkylaminoalkoxy” means —O-(alkyl)-NH(alkyl) or —O-(alkyl)-N(alkyl)(alkyl), respectively, such as —OCH₂NHCH₃, —OCH₂CH₂N(CH₃)₂, and the like

The term “arylamino” means —NH(aryl), such as —NH-phenyl, —NH-pyridinyl, and the like.

The term “arylalkylamino” means —NH-(alkyl)-(aryl), such as —NH-benzyl, —NHCH₂— pyridinyl, and the like.

The term “alkylamino” means —NH(alkyl), such as —NHCH₃, —NHCH₂CH₃, and the like.

The term “cycloalkylamino” means —NH-(cycloalkyl), such as —NH-cyclohexyl, and the like.

The term “cycloalkylalkylamino”-NH-(alkyl)-(cycloalkyl), such as —NHCH₂— cyclohexyl, and the like.

It is noted in regard to all of the definitions provided herein that the definitions should be interpreted as being open ended in the sense that further substituents beyond those specified can be included. Hence, a C₁ alkyl indicates that there is one carbon atom but does not indicate what are the substituents on the carbon atom. Hence, a C₁ alkyl comprises methyl (i.e., —CH₃) as well as —CRₐR_{b}R_{c} where Rₐ, R_{b}, and R_{c} can each independently be hydrogen or any other substituent where the atom alpha to the carbon is a heteroatom or cyano. Hence, CF₃, CH₂OH and CH₂CN are all C₁ alkyls.

The term “derivative” as used herein refers to a chemical substance related structurally to another, i.e., an “original” substance, which can be referred to as a “parent” compound. A “derivative” can be made from the structurally-related parent compound in one or more steps. In some embodiments, the general physical and chemical properties of a derivative can be similar to or different from the parent compound.

Unless otherwise stated, structures depicted herein are meant to include compounds which differ only in the presence of one or more isotopically enriched atoms. For example, compounds having the present structure except for the replacement of a hydrogen atom by a deuterium or tritium, or the replacement of a carbon atom by a ¹³C- or ¹⁴C-enriched carbon are within the scope of the invention.

A “pharmaceutically acceptable salt”, as used herein, is intended to encompass any compound described herein that is utilized in the form of a salt thereof, especially where the salt confers on the compound improved pharmacokinetic properties as compared to the free form of compound or a different salt form of the compound. The pharmaceutically acceptable salt form can also initially confer desirable pharmacokinetic properties on the compound that it did not previously possess, and may even positively affect the pharmacodynamics of the compound with respect to its therapeutic activity in the body. An example of a pharmacokinetic property that can be favorably affected is the manner in which the compound is transported across cell membranes, which in turn may directly and positively affect the absorption, distribution, biotransformation and excretion of the compound. While the route of administration of the pharmaceutical composition is important, and various anatomical, physiological and pathological factors can critically affect bioavailability, the solubility of the compound is usually dependent upon the character of the particular salt form thereof, which it utilized. One of skill in the art will appreciate that an aqueous solution of the compound will provide the most rapid absorption of the compound into the body of a subject being treated, while lipid solutions and suspensions, as well as solid dosage forms, will result in less rapid absorption of the compound.

Pharmaceutically acceptable salts include those derived from inorganic acids such as sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like. See, for example, Berge et al., “Pharmaceutical Salts”, J. Pharm. Sci. 66:1-19 (1977), the content of which is herein incorporated by reference in its entirety.

Exemplary salts also include the hydrobromide, hydrochloride, sulfate, bisulfate, phosphate, nitrate, acetate, succinate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. Suitable acids which are capable of forming salts with the compounds of the disclosure include inorganic acids such as hydrochloric acid, hydrobromic acid, perchloric acid, nitric acid, thiocyanic acid, sulfuric acid, phosphoric acid, and the like; and organic acids such as 1,2-ethanedisulfonic acid, 2-hydroxyethanesulfonic acid, 2-naphthalenesulfonic acid, 3-phenylpropionic acid, 4-methylbicyclo[2.2.2]oct-2-ene-1-carboxylic acid, 4,4′-mefhylenebis(3-hydroxy-2-ene-1-carboxylic acid), acetic acid, anthranilic acid, benzenesulfonic acid, benzoic acid, camphorsulfonic acid, cinnamic acid, citric acid, cyclopentanepropionic acid, ethanesulfonic acid, formic acid, fumaric acid, glucoheptonic acid, gluconic acid, glutamic acid, glycolic acid, heptanoic acid, hydroxynaphthoic acid, lactic acid, lauryl sulfuric acid, maleic acid, malic acid, malonic acid, mandelic acid, methanesulfonic acid, muconic acid, naphthalene sulfonic acid, o-(4-hydroxybenzoyl)benzoic acid, oxalic acid, p-chlorobenzenesulfonic acid, propionic acid, p-toluenesulfonic acid, pyruvic acid, salicylic acid, stearic acid, succinic acid, sulfanilic acid, tartaric acid, tertiary butylacetic acid, trifluoroacetic acid, trimethylacetic acid, and the like. Suitable bases capable of forming salts with the compounds of the disclosure include inorganic bases such as sodium hydroxide, ammonium hydroxide, sodium carbonate, calcium hydroxide, potassium hydroxide and the like; and organic bases such as mono-, di- and tri-alkyl and aryl amines (e.g., triethylamine, diisopropyl amine, methyl amine, dimethyl amine, N-methylglucamine, pyridine, picoline, dicyclohexylamine, N,N′-dibezylethylenediamine, and the like), and optionally substituted ethanol-amines (e.g., ethanolamine, diethanolamine, trierhanolamine and the like).

In some embodiments, the compounds described herein can be in the form of a prodrug. The term “prodrug” as used herein refers to compounds that can be converted via some chemical or physiological process (e.g., enzymatic processes and metabolic hydrolysis) to compound described herein. Thus, the term “prodrug” also refers to a precursor of a biologically active compound that is pharmaceutically acceptable. A prodrug can be inactive when administered to a subject, i.e. an ester, but is converted in vivo to an active compound, for example, by hydrolysis to the free carboxylic acid or free hydroxyl. The prodrug compound often offers advantages of solubility, tissue compatibility or delayed release in an organism. The term “prodrug” is also meant to include any covalently bonded carriers, which release the active compound in vivo when such prodrug is administered to a subject. Prodrugs of an active compound, as described herein, may be prepared by modifying functional groups present in the active compound in such a way that the modifications are cleaved, either in routine manipulation or in vivo, to the parent active compound. Prodrugs include compounds wherein a hydroxy, amino or mercapto group is bonded to any group that, when the prodrug of the active compound is administered to a subject, cleaves to form a free hydroxy, free amino or free mercapto group, respectively. For example, a compound comprising a hydroxy group can be administered as an ester that is converted by hydrolysis in vivo to the hydroxy compound. Suitable esters that can be converted in vivo into hydroxy compounds include acetates, citrates, lactates, tartrates, malonates, oxalates, salicylates, propionates, succinates, fumarates, formates, benzoates, maleates, methylene-bis-b-hydroxynaphthoates, gentisates, isethionates, di-p-toluoyltartrates, methanesulfonates, ethanesulfonates, benzenesulfonates, p-toluenesulfonates, cyclohexylsulfamates, quinates, esters of amino acids, and the like. Similarly, a compound comprising an amine group can be administered as an amide, e.g., acetamide, fornmamide and benzamide that is converted by hydrolysis in vivo to the amine compound. See Harper, “Drug Latentiation” in Jucker, ed. Progress in Drug Research 4:221-294 (1962); Morozowich et al, “Application of Physical Organic Principles to Prodrug Design” in E. B. Roche ed. Design of Biopharmaceutical Properties through Prodrugs and Analogs, APHA Acad. Pharm. Sci. 40 (1977); Bioreversible Carriers in Drug in Drug Design, Theory and Application, E. B. Roche, ed., APHA Acad. Pharm. Sci. (1987); Design of Prodrugs, H. Bundgaard, Elsevier (1985); Wang et al. “Prodrug approaches to the improved delivery of peptide drug” in Curr. Pharm. Design. 5(4):265-287 (1999); Pauletti et al. (1997) Improvement in peptide bioavailability: Peptidomimetics and Prodrug Strategies, Adv. Drug. Delivery Rev. 27:235-256; Mizen et al. (1998) “The Use of Esters as Prodrugs for Oral Delivery of (3-Lactam antibiotics,” Pharm. Biotech. 11:345-365; Gaignault et al. (1996) “Designing Prodrugs and Bioprecursors I. Carrier Prodrugs,” Pract. Med. Chem. 671-696; Asgharnejad, “Improving Oral Drug Transport”, in Transport Processes in Pharmaceutical Systems, G. L. Amidon, P. I. Lee and E. M. Topp, Eds., Marcell Dekker, p. 185-218 (2000); Balant et al., “Prodrugs for the improvement of drug absorption via different routes of administration”, Eur. J. Drug Metab. Pharmacokinet., 15(2): 143-53 (1990); Balimane and Sinko, “Involvement of multiple transporters in the oral absorption of nucleoside analogues”, Adv. Drug Delivery Rev., 39(1-3): 183-209 (1999); Browne, “Fosphenytoin (Cerebyx)”, Clin. Neurophannrmacol. 20(1): 1-12 (1997); Bundgaard, “Bioreversible derivatization of drugs—principle and applicability to improve the therapeutic effects of drugs”, Arch. Pharm. Chemi 86(1): 1-39 (1979); Bundgaard H. “Improved drug delivery by the prodrug approach”, Controlled Drug Delivery 17: 179-96 (1987); Bundgaard H. “Prodrugs as a means to improve the delivery of peptide drugs”, Arfv. Drug Delivery Rev. 8(1): 1-38 (1992); Fleisher et al. “Improved oral drug delivery: solubility limitations overcome by the use of prodrugs”, Arfv. Drug Delivery Rev. 19(2): 115-130 (1996); Fleisher et al. “Design of prodrugs for improved gastrointestinal absorption by intestinal enzyme targeting”, Methods Enzymol. 112 (Drug Enzyme Targeting, Pt. A): 360-81, (1985); Farquhar D, et al., “Biologically Reversible Phosphate-Protective Groups”, Pharm. Sci., 72(3): 324-325 (1983); Freeman S, et al., “Bioreversible Protection for the Phospho Group: Chemical Stability and Bioactivation of Di(4-acetoxy-benzyl) Methylphosphonate with Carboxyesterase,” Chem. Soc., Chem. Commun., 875-877 (1991); Friis and Bundgaard, “Prodrugs of phosphates and phosphonates: Novel lipophilic alphaacyloxyalkyl ester derivatives of phosphate- or phosphonate containing drugs masking the negative charges of these groups”, Eur. J. Pharm. Sci. 4: 49-59 (1996); Gangwar et al., “Pro-drug, molecular structure and percutaneous delivery”, Des. Biopharm. Prop. Prodrugs Analogs, [Symp.] Meeting Date 1976, 409-21. (1977); Nathwani and Wood, “Penicillins: a current review of their clinical pharmacology and therapeutic use”, Drugs 45(6): 866-94 (1993); Sinhababu and Thakker, “Prodrugs of anticancer agents”, Adv. Drug Delivery Rev. 19(2): 241-273 (1996); Stella et al., “Prodrugs. Do they have advantages in clinical practice?”, Drugs 29(5): 455-73 (1985); Tan et al. “Development and optimization of anti-HIV nucleoside analogs and prodrugs: A review of their cellular pharmacology, structure-activity relationships and pharmacokinetics”, Adv. Drug Delivery Rev. 39(1-3): 117-151 (1999); Taylor, “Improved passive oral drug delivery via prodrugs”, Adv. Drug Delivery Rev., 19(2): 131-148 (1996); Valentino and Borchardt, “Prodrug strategies to enhance the intestinal absorption of peptides”, Drug Discovery Today 2(4): 148-155 (1997); Wiebe and Knaus, “Concepts for the design of anti-HIV nucleoside prodrugs for treating cephalic HIV infection”, Adv. Drug Delivery Rev.: 39(1-3):63-80 (1999); Waller et al., “Prodrugs”, Br. J. Clin. Pharmac. 28: 497-507 (1989), content of all of which are herein incorporated by reference in its entirety.

The term “protected derivatives” means derivatives of compounds described herein in which a reactive site or sites are blocked with protecting groups. Protected derivatives are useful in the preparation of compounds or in themselves can be active. A comprehensive list of suitable protecting groups can be found in T. W. Greene, Protecting Groups in Organic Synthesis, 3rd edition, John Wiley & Sons, Inc. 1999.

“Isomers” mean any compound having identical molecular formulae but differing in the nature or sequence of bonding of their atoms or in the arrangement of their atoms in space. Isomers that differ in the arrangement of their atoms in space are termed “stereoisomers”. Stereoisomers that are not mirror images of one another are termed “diastereomers” and stereoisomers that are nonsuperimposable mirror images are termed “enantiomers” or sometimes “optical isomers”. A carbon atom bonded to four nonidentical substituents is termed a “chiral center”. A compound with one chiral center has two enantiomeric forms of opposite chirality. A mixture of the two enantiomeric forms is termed a “racemic mixture”. A compound that has more than one chiral center has 2ⁿ⁻¹ enantiomeric pairs, where n is the number of chiral centers. Compounds with more than one chiral center may exist as ether an individual diastereomers or as a mixture of diastereomers, termed a “diastereomeric mixture”. When one chiral center is present a stereoisomer may be characterized by the absolute configuration of that chiral center. Absolute configuration refers to the arrangement in space of the substituents attached to the chiral center. Enantiomers are characterized by the absolute configuration of their chiral centers and described by the R- and S-sequencing rules of Cahn, Ingold and Prelog. Conventions for stereochemical nomenclature, methods for the determination of stereochemistry and the separation of stereoisomers are well known in the art (e.g., see “Advanced Organic Chemistry”, 4th edition, March, Jerry, John Wiley & Sons, New York, 1992).

The term “enantiomer” is used to describe one of a pair of molecular isomers which are mirror images of each other and non-superimposable. Other terms used to designate or refer to enantiomers include “stereoisomers” (because of the different arrangement or stereochemistry around the chiral center; although all enantiomers are stereoisomers, not all stereoisomers are enantiomers) or “optical isomers” (because of the optical activity of pure enantiomers, which is the ability of different pure enantiomers to rotate planepolarized light in different directions). Enantiomers generally have identical physical properties, such as melting points and boiling points, and also have identical spectroscopic properties. Enantiomers can differ from each other with respect to their interaction with plane-polarized light and with respect to biological activity.

The designations “R” and “S” are used to denote the absolute configuration of the molecule about its chiral center(s). The designations may appear as a prefix or as a suffix; they may or may not be separated from the isomer by a hyphen; they may or may not be hyphenated; and they may or may not be surrounded by parentheses.

The designations or prefixes “(+)” and “(−)” are employed to designate the sign of rotation of plane-polarized light by the compound, with (−) meaning that the compound is levorotatory (rotates to the left). A compound prefixed with (+) is dextrorotatory (rotates to the right).

The term “racemic mixture,” “racemic compound” or “racenmate” refers to a mixture of the two enantiomers of one compound. An ideal racemic mixture is one wherein there is a 50:50 mixture of both enantiomers of a compound such that the optical rotation of the (+) enantiomer cancels out the optical rotation of the (−) enantiomer.

The term “resolving” or “resolution” when used in reference to a racemic mixture refers to the separation of a racemate into its two enantiomorphic forms (i.e., (+) and (−); 65 (R) and (S) forms). The terms can also refer to enantioselective conversion of one isomer of a racenmate to a product.

The term “enantiomeric excess” or “ee” refers to a reaction product wherein one enantiomer is produced in excess of the other, and is defined for a mixture of (+)- and (−)-enantiomers, with composition given as the mole or weight or volume fraction F(+) and F(−) (where the sum of F(+) and F(−)=1). The enantiomeric excess is defined as * F(+)−F(−)* and the percent enantiomeric excess by 100x*F(+)−F(−)*. The “purity” of an enantiomer is described by its ee or percent ee value (% ee).

Whether expressed as a “purified enantiomer” or a “pure enantiomer” or a “resolved enantiomer” or “a compound in enantiomeric excess”, the terms are meant to indicate that the amount of one enantiomer exceeds the amount of the other. Thus, when referring to an enantiomer preparation, both (or either) of the percent of the major enantiomer (e.g. by mole or by weight or by volume) and (or) the percent enantiomeric excess of the major enantiomer may be used to determine whether the preparation represents a purified enantiomer preparation.

The term “enantiomeric purity” or “enantiomer purity” of an isomer refers to a qualitative or quantitative measure of the purified enantiomer; typically, the measurement is expressed on the basis of ee or enantiomeric excess.

The terms “substantially purified enantiomer,” “substantially resolved enantiomer” “substantially purified enantiomer preparation” are meant to indicate a preparation (e.g. derived from non-optically active starting material, substrate, or intermediate) wherein one enantiomer has been enriched over the other, and more preferably, wherein the other enantiomer represents less than 20%, more preferably less than 10%, and more preferably less than 5%, and still more preferably, less than 2% of the enantiomer or enantiomer preparation.

The terms “purified enantiomer,” “resolved enantiomer” and “purified enantiomer preparation” are meant to indicate a preparation (e.g. derived from non-optically active starting material, substrates or intermediates) wherein one enantiomer (for example, the R-enantiomer) is enriched over the other, and more preferably, wherein the other enantiomer (for example the S-enantiomer) represents less than 30%, preferably less than 20%, more preferably less than 10% (e.g. in this particular instance, the R-enantiomer is substantially free of the S-enantiomer), and more preferably less than 5% and still more preferably, less than 2% of the preparation. A purified enantiomer may be synthesized substantially free of the other enantiomer, or a purified enantiomer may be synthesized in a stereo-preferred procedure, followed by separation steps, or a purified enantiomer may be derived from a racemic mixture.

The term “enantioselectivity,” also called the enantiomeric ratio indicated by the symbol “E,” refers to the selective capacity of an enzyme to generate from a racemic substrate one enantiomer relative to the other in a product racemic mixture; in other words, it is a measure of the ability of the enzyme to distinguish between enantiomers. A nonselective reaction has an E of 1, while resolutions with E's above 20 are generally considered useful for synthesis or resolution. The enantioselectivity resides in a difference in conversion rates between the enantiomers in question. Reaction products are obtained that are enriched in one of the enantiomers; conversely, remaining substrates are enriched in the other enantiomer. For practical purposes it is generally desirable for one of the enantiomers to be obtained in large excess. This is achieved by terminating the conversion process at a certain degree of conversion.

### BRIEF DESCRIPTION OF THE DRAWINGS

This patent or application file contains at least one drawing executed in color. Copies of this patent or patent application publication with color drawing (s) will be provided by the Office upon request and payment of the necessary fee.

FIG. 1 shows results from experiments that indicate the compounds identified from the screen inhibit purified DsbB.

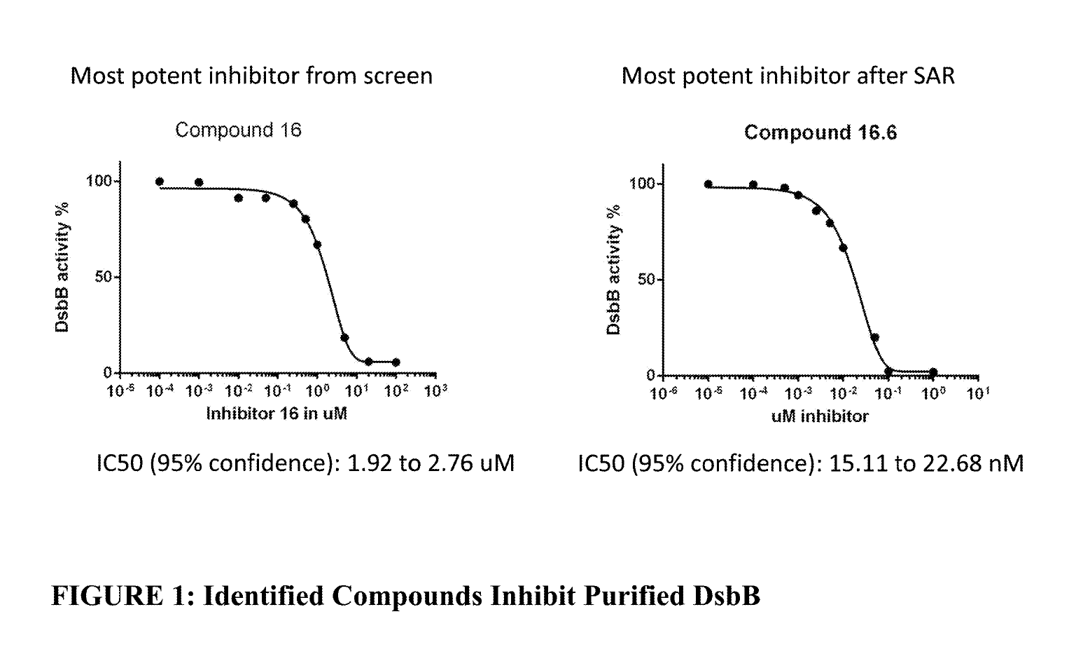

FIG. 2 shows results from experiments that indicate E. coli dsbB inhibitors also inhibit dsbB from other gram-negative bacteria

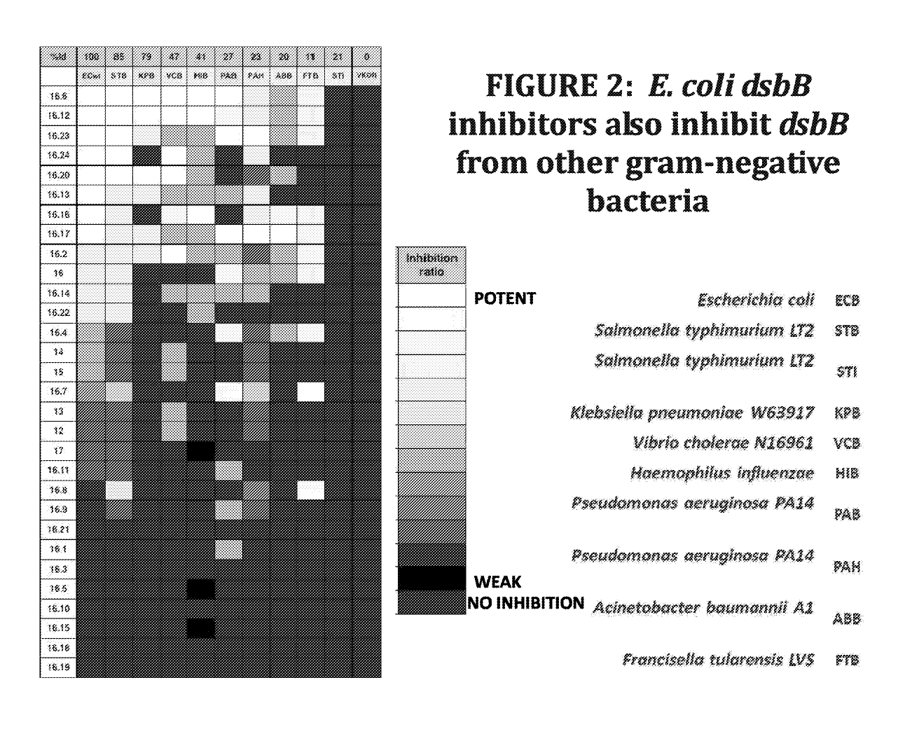

FIG. 3 shows results from experiments that indicate the strongest inhibitor identified also impairs twitching motility of Pseudomonas aeruginosa.

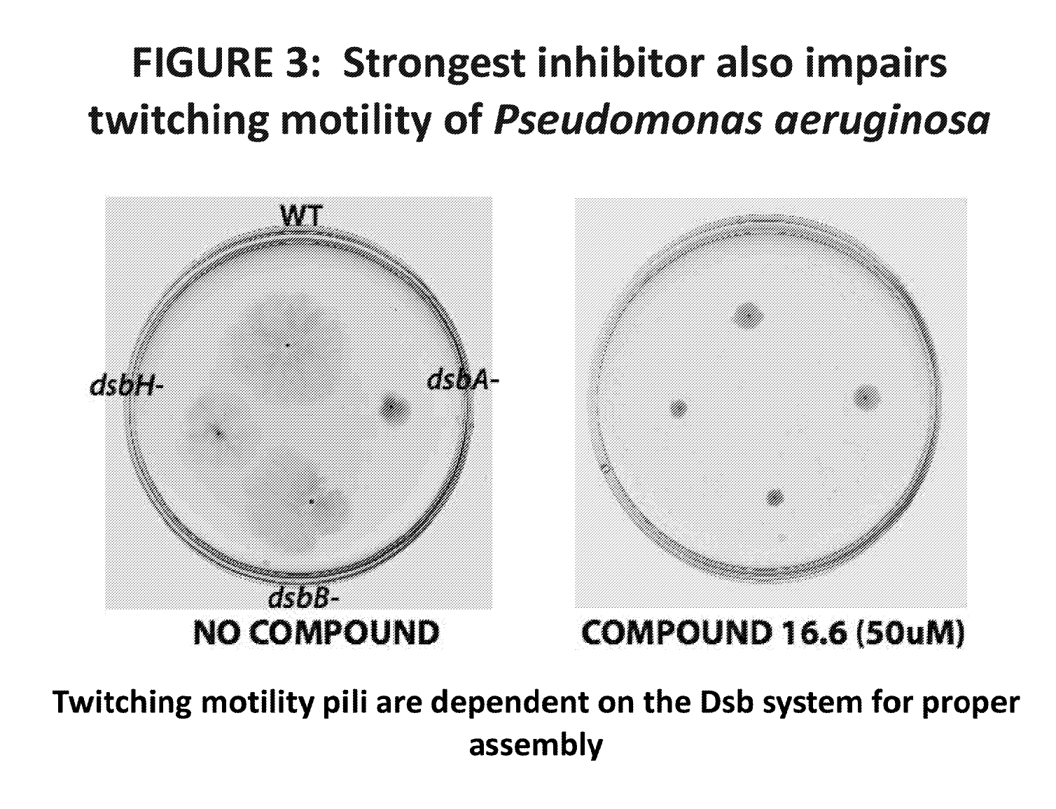

FIG. 4 shows results from experiments that indicate inhibition of other DsbB enzymes from gram-negative bacteria by EcDsbB inhibitors.

FIG. 5 contains dose response curves for inhibition of purified EcDsbB. In vitro inhibition experiments of purified EcDsbB enzyme by compounds 16 (left) and 16.6 (right) were performed. The results shown are an average of at least two independent experiments ±s.d. This figure is an update of FIG. 1, with error bars.

FIG. 6A-FIG. 6B contains dixon plots of DsbB activity (A) with compound 16, values represent the average of three independent experiments and (B) with compound 16.6, values represent the average of two independent experiments.

FIG. 7A-FIG. 7C shows experimental results that indicate the mechanism of inhibition by compound 16.6. (A) In vivo accumulation of reduced DsbB when incubating cells with compound 16.6. Cells were grown aerobically with different concentrations of drug and precipitated proteins were treated with Maleimide-PEG2k (ME2k, 2 kDa). Samples were run on reducing SDS-PAGE and immunoblotted against anti-DsbB. Dithiothreitol (DTF) was used for reducing disulfide bonds prior to alkylation. “oxidized” refers to the position of the oxidized protein which is the same as that of the protein with all four cysteines (Cys) mutated. “Reduced” refers to bands where the positions of the protein with the four or indicated number of reduced cysteines are detected due to alkylation which adds to the molecular weight. Gel shown is a representative immunoblot of two independent experiments. (B) Visible absorbance spectra of DsbB and DsbB-DsbAC33A dimer. The pink color of the DsbB-ubiquinone charge-transfer complex diminishes when compound 16.6 is added, indicating disruption of the interaction between Cys44 of DsbB and the cofactor ubiquinone. DsbB or DsbB-DsbAC33A complex (each at 100 μM) were mixed with compound 16.6 (or with DMSO) at 1:2 molar ratio in 50 mM Tris buffer pH 8.0 containing 300 mM NaCl and 0.05% DDM. Samples were incubated on ice for about 4 minutes before the spectra were recorded using 1 cm quarts cuvettes. (C) Summary of deconvoluted masses obtained from ESI-MS analysis of proteins treated with compound 16.6 (last column). MS/MS fragmentation of DsbB peptide C*IYERVAL (SEQ ID NO: 1). Sequencing ions of the modified (44-51)-peptide was performed and gave information consistent with modification of Cys44 by compound 16.6. The calculated monoisotopic mass of modified b5 ion (residues 44-48, CIYER) is 917.293 Da and the observed mass is 917.295 Da. The calculated mass of the unmodified peptide is 664.300 Da. Thus the mass difference is 252.995 Da which is in agreement with the loss of a chloride ion from 16.6 upon binding to Cys44, 287.962 (mass of compound 16.6)−34.969 (mass of chloride ion)=252.993 Da.

FIG. 8 is a table of experimental results that indicates In vivo inhibition of DsbB enzymes from Gram-negative bacteria expressed in E. coli. E. coli (Ec) dsbB mutant strains expressing β-Gal^{dbs} and dsbB genes from Salmonella typhimurium (St), Klebsiella pneumoniae (Kp), Vibrio cholerae (Vc), Haernophilus influenzae (Hi), Pseudomonas aeruginosa (Pa), Acinetobacter baumannii (Ab) and Francisella tularensis (Ft), as well as two DsbB homologs from P. aeruginosa (dsbH) and S. typhimurium (dsbl) and a non-homolog vkor from Mycobacterium tuberculosis (Mtb), were tested against the pyridazinone-like compounds listed on the left of the table. Inhibition range from strong to weak is relative to each DsbB-expressing strain and was obtained by dividing the MIC of each compound by the lowest MIC observed for each particular strain. Results are the average of three independent experiments. Compounds that did not inhibit at the highest concentration tested are shown as black. The table shown in grayscale was adapted from a color table, rating the specified inhibitors of the indicated bacterial DsbB enzyme, by light to dark coding, strong (light) to weak (grey) to non-inhibitors (black).

FIG. 9 is a bar graph of experimental results that indicate the inhibition of DsbB homologs in Pseudomonas aeruginosa PA14.

### DETAILED DESCRIPTION OF THE INVENTION

Aspects of the invention are based on the identification of compounds that inhibit DsbB in bacteria, and the further determination that such compounds affect the virulence and/or growth of the microbes. In addition, the compounds potentiate the inhibitory activity of other agents. These activities indicate that the identified compounds can be formulated into compositions for inhibiting microbe virulence and/or growth Such formulations may be pharmaceutical compositions for in vivo uses (e.g., administration to a subject), or may be formulated for in vitro uses to inhibit or eliminate bacterial contamination. Such compositions may contain an effective amount of one or more of the identified compounds, and may also contain effective amount of additional agents with antimicrobial activity. Examples of such additional agents for use in combination with the identified compounds are discussed herein.

One aspect of the invention relates to a novel composition (e.g., pharmaceutical and/or antibacterial) comprising a compound of Formula I:

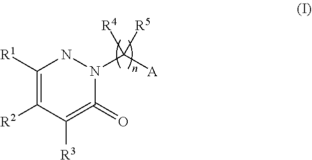

wherein

R¹, R² and R³ are independently selected from the group consisting of hydrogen, deuterium, halogen, cyano, optionally substituted alkyl, optionally substituted cyclyl, optionally substituted heterocyclyl, optionally substituted aryl, optionally substituted heteroaryl, OR⁶, CO₂R⁶, C(O)NR⁶R⁷, OC(O)R⁶, N(R⁶)C(O)R⁶, NR⁶R⁷, SR⁶, S(O)—R⁶, SO₂R⁶, OS(O)₂R⁶, SO₂NR⁶NR⁷, and NO₂;

R⁴ and R⁵ are independently hydrogen, deuterium, optionally substituted alkyl, or halogen, or R⁴ and R⁵ together with the carbon they are attached to form an optionally substituted cyclic alkyl or optionally substituted heterocyclic;

R⁶ and R⁷ are independently for each occurrence hydrogen, optionally substituted alkyl, optionally substituted cyclyl, optionally substituted heterocyclyl, optionally substituted aryl, or optionally substituted heteroaryl; A is aryl, heteroaryl, cyclyl, heterocyclyl, or alkyl, each of which can be optionally substituted; and

n is 0, 1, or 2.

In some embodiments at least one (e.g., one, two, or three) of R¹, R² and R³ is independently hydrogen, halogen, NO₂, OS(O)₂R⁶, cyano, hydroxyl, alkoxy, alkylthio, alkylamino, heterocyclyl, or alkyl.

In some embodiments, at least one (e.g., one, two, or three) of R¹, R² and R³ is hydrogen. In some embodiments, R¹ is hydrogen. In some embodiments, R² is hydrogen. In some embodiments, R³ is hydrogen.

In some embodiments, at least one (e.g., one, two, or three) of R¹, R² and R³ is halogen. In some embodiments, R¹ is a halogen. In some embodiments, R² is a halogen. In some embodiments, R³ is a halogen. In some embodiments, R² and R³ are independently selected halogen.

In some embodiments, at least one (e.g., one, two, or three) of R¹, R² and R³ is hydroxyl. In some embodiments, R¹ is hydroxyl. In some embodiments, R² is hydroxyl. In some embodiments, R³ is hydroxyl.

In some embodiments, at least one (e.g., one, two, or three) of R¹, R² and R³ is an alkoxy. In some embodiments, R¹ is alkoxy. In some embodiments, R² is alkoxy. In some embodiments, R³ is alkoxy. In some embodiments, R² and R³ are independently selected alkoxy.

In some embodiments, at least one (e.g., one, two, or three) of R¹, R² and R³ is an optionally substituted heterocyclyl. In one embodiment, R¹ is an optionally substituted heterocyclyl. In one embodiment, R² is an optionally substituted heterocyclyl. In one embodiment, R³ is an optionally substituted heterocyclyl.

In some embodiments, at least one (e.g., one, two, or three) of R¹, R² and R³ is an alkylthio. In some embodiments, R¹ is alkylthio. In some embodiments, R² is alkylthio. In some embodiments, R³ is alkylthio. In some embodiments, R² and R³ are independently selected alkylthio.

In some embodiments, at least one (e.g., one, two, or three) of R¹, R² and R³ is an alkylamino. In some embodiments, R¹ is alkylamino. In some embodiments, R² is alkylamino. In some embodiments, R³ is alkylamino.

In some embodiments, at least one (e.g., one, two, or three) of R¹, R² and R³ is an optionally substituted alkyl. In some embodiments, R¹ is alkyl. In some embodiments, R² is alkyl. In some embodiments, R³ is alkyl.

In some embodiments, R² is a halogen, NO₂, OS(O)₂R⁶, cyano, hydroxyl, alkoxy, or alkylthio; and R³ is a halogen; heterocyclyl; hydroxyl, alkoxy, or alkylthio.

In some embodiments, R¹ is hydrogen; R² is a halogen, NO₂, OS(O)₂R⁶, cyano, hydroxyl, alkoxy, or alkylthio; and R³ is a halogen; heterocyclyl; hydroxyl, alkoxy, or alkylthio.

In some embodiments, R² is Cl, Br, I, F, NO₂, OH, methoxy (—OCH₃), ethoxy (—OEt), mesylate (—OS(O)₂Me), triflate (—OS(O)₂CF₃), besylate (—OS(O)₂Ph), tosylate (—OS(O)₂C6H₄CH₃), methylthio (—SCH₃), or ethylthio (—SCH₂CH₃).

In some embodiments, R³ is Cl, Br, optionally pyrrolidinyl, methoxy, ethoxy (—OCH₂CH₃) or butylamino (—NH(CH₂)₃CH₃).

In some embodiments, R² is Cl, and R³ is Cl, methoxy, ethoxy, pyrrolidinyl, or butylamino; R² is hydroxyl, methoxy, or ethykhio, and R³ is Cl; R² and R³ are both Br; or R² and R³ are both methylthio.

In one embodiment, R¹ is hydrogen, and R² is Cl, and R³ is Cl, methoxy, ethoxy, pyrrolidinyl, or butylamino; R¹ is hydrogen, and R² is hydroxyl, methoxy, or ethylthio, and R³ is Cl; R¹ is hydrogen, and R² and R³ are both Br; or R¹ is hydrogen, and R² and R³ are both methylthio.

Variable n is independently 0, 1, or 2. In one embodiment, n is 0. In another embodiment, n is 1.

In some embodiments, R⁴ and R⁵ are the same. In some embodiments, R⁴ and R⁵ are different. In some embodiments, R⁴ and R⁵ are selected independently from hydrogen, deuterium, optionally substituted C₁-C₆alkyl, and halogen. In some embodiments, R⁴ and R⁵, together with the carbon they are attached to, form an optionally substituted C3-C8 cyclic alkyl. In some embodiments, R⁴ and R⁵, together with the carbon they are attached to, form an optionally substituted 3-6 membered heterocyclic (or heterocyclyl). In some embodiments, at least one of R⁴ and R⁵ is hydrogen. In some embodiments, both R⁴ and R⁵ are hydrogen.

In one embodiment, n is 1 and both R⁴ and R⁵ are hydrogen.

In some embodiments, A is an optionally substituted alkyl, optionally substituted aryl or optionally substituted heteroaryl.

In some embodiments, A is an optionally substituted C₁-C₁₂alkyl, optionally substituted C₁-C₁₀alkyl, optionally substituted C₁-C₈alkyl, or optionally substituted C₁-C₆alkyl. In some embodiment, A is methyl, ethyl, n-propyl, i-propyl, n-butyl, i-butyl, or t-butyl. In one embodiment, A is methyl.

In some embodiments, A is an aryl or heteroaryl optionally substituted with one or more (e.g., one, two, three, four, five, six, seven, eight, nine or more) substituents selected independently from deuterium, C₁-C₆alkyl, OR¹⁴, N(R¹⁴)R¹⁵, C(O)OR¹⁴, C(O)N(R¹⁴)R¹⁵, SO₂NR¹⁴NR¹⁵, C3-C8 cyclic alkyl, 3-6 membered heterocyclyl, aryl, and heteroaryl.

In some embodiments, A is an optionally substituted aryl of structure

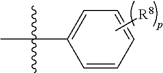

wherein R⁸ is independently for each occurrence deuterium, halogen, cyano, optionally substituted alkyl, optionally substituted cyclyl, optionally substituted heterocyclyl, optionally substituted aryl, optionally substituted heteroaryl, OR⁹, CO₂R⁹, C(O)NR⁹R¹⁰, OC(O)R⁹, N(R⁹)C(O)R⁹, NR⁹R¹⁰, SR⁹, S(O)R⁹, SO₂R⁹, SO₂NR⁹NR¹⁰, and NO₂ and p is 0, 1, 2, 3, 4, or 5, wherein R⁹ and R¹⁰ are independently for each occurrence hydrogen, optionally substituted alkyl, optionally substituted cyclyl, optionally substituted heterocyclyl, optionally substituted aryl, or optionally substituted heteroaryl.

In one embodiment, p is 0. In another embodiment, p is 1. In yet another embodiment, p is 1. In still yet another embodiment, p is 2. Accordingly, in some embodiments, the optionally substituted aryl is phenyl; 2-substituted phenyl; 3-substituted phenyl; 2,6-disubstituted phenyl, wherein substituents at the 2-position and 6-position are independently selected; 4-substituted phenyl;’ or 2,3,6-trisubstituted phenyl, wherein substituents at the 2-, 3-, and 6-positions are independently selected

In some embodiments, each R⁸ is independently halogen, optionally substituted alkyl, hydroxyl, alkoxy, alkylthio, CF₃, OCF₃, C(O)OR⁹, C(O)NR⁹R¹⁰, NO₂, or CN. In some embodiments, each R⁸ is independently bromo, chloro, fluoro, methyl, methoxy, CN, NO₂, C(O)NH₂, or C(O)OMe.

In some embodiments, A is an optionally substituted naphthalene of structure

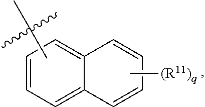

wherein R¹¹ independently for each occurrence deuterium, halogen, cyano, optionally substituted alkyl, optionally substituted cyclyl, optionally substituted heterocyclyl, optionally substituted aryl, optionally substituted heteroaryl, OR², C(O)OR¹³, C(O)NR¹²R¹³, OC(O)R¹², N(R¹²)C(O)R¹², NR¹²R¹³, SR¹², S(O)R¹², SO₂R¹², SO₂NR¹²NR¹³, and NO₂; and q is 0, 1, 2, 3, 4, 5, 6, or 7, wherein R¹² and R¹³ are independently for each occurrence hydrogen, optionally substituted alkyl, optionally substituted cyclyl, optionally substituted heterocyclyl, optionally substituted aryl, or optionally substituted heteroaryl. In one embodiment, the optionally substituted naphthalene is

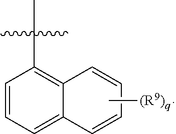

In one embodiment, q is 0. In another embodiment, q is 1.

In some embodiments, A is an optionally substituted heteroaryl containing 1, 2, 3, or 4 independently selected heteroatoms. In some embodiments, A is an optionally substituted heteroaryl containing 1-2 sulfur atoms. In some embodiments, A is an optionally substituted heteroaryl containing 1-4 nitrogen atoms. In some embodiments, A is an optionally substituted heteroaryl containing 1-2 oxygen atoms.

In some embodiments, A is an optionally substituted pyrimidine. In one embodiment, the optionally substituted pyrimidine is 4,6-disubstitutedpyrimidin-2-yl.

In some embodiments, A is an optionally substituted thiophene. Accordingly, in some embodiments, the optionally substituted thiophene is a 2-substituted thiophene, 3-substituted thiophene, 4-substituted thiophene, 5-substituted thiophene, 2,4-substituted thiophene, 2,5-substituted thiophene, 3,4-substituted thiophene, 3,5-substituted thiophene, or 4,5-substituted thiophene, wherein the substituents at the 2-, 3-, 4-, and 5-positions are independently selected. In some embodiments, the optionally substituted thiophene is substituted with one or more halogens independently selected from F, Cl, Br and I.

In some embodiments, A is an optionally substituted pyridine. Accordingly, in some embodiments, the optionally substituted pyridine is a 2-substituted pyridine, 3-substituted pyridine, 4-substituted pyridine, 5-substituted pyridine, 6-substituted pyridine, 2,3-substituted pyridine, 2,4-substituted pyridine, 2,5-substituted pyridine, 2,6-substituted pyridine, 3,4-substituted pyridine, 3,5-substituted pyridine, 3,6-substituted pyridine, 4,5-substituted pyridine, 4,6-substituted pyridine, or 5,6-substituted pyridine, wherein the substituents at the 2-, 3-, 4-, 5- and 6-positions are independently selected. In some embodiments, the optionally substituted pyridine is substituted with one or more halogens independently selected from F, Cl, Br and I.

In one embodiment, A is selected from the group consisting of methyl, phenyl; 2-bromophenyl; 2-fluorophenyl; 2-chlorophenyl; 2-methylphenyl; 3-methylphenyl; 2-nitrophenyl; 2-cyanophenyl; 2-chloro-6-fluorophenyl; 4-nitrophenyl; 4-chlorophenyl; 4-bromophenyl; 3-methoxyphenyl; 3-cyanophenyl; 2,3,6-trichlorophenyl; 4-aminoformylphenyl; 4-methoxycarbonylphenyl; 2-trifluoromethylphenyl; 2-trifluoromethoxyphenyl; thiophen-2-yl; 3-chlorothiophen-2-yl; pyridin-2-yl; 3-chloropyridin-2-yl; pyridine-4-yl; 3-chloropyridin-4-yl; naphthalen-1-yl; or 4,6-dimethylpyrimidin-2-yl.

In some embodiments, a compound of Formula I is of Formula II:

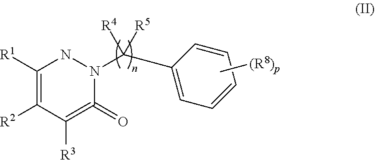

wherein variables are as defined above.

In some embodiments, a compound of Formula II is of Formula II′:

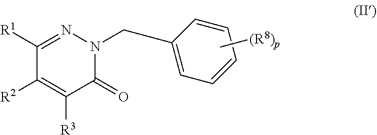

wherein variables are as defined above.

In some other embodiments, a compound of formula II is of formula II″:

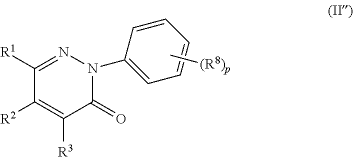

In some embodiments, a compound of Formula I is of Formula III:

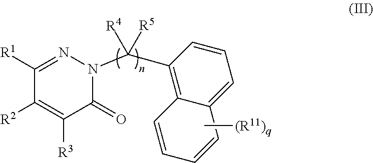

wherein variables are as defined above.

In some embodiments, a compound of formula III is of formula III′:

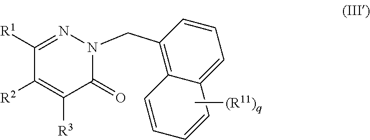

wherein variables are as defined above.

In one embodiment the compound of Formula I, II or III is selected from Table 1.

TABLE 1

| Compound | Structure |
|---|---|
| C16.6 | 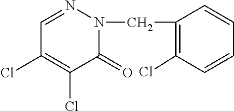 [SMILES: O=c1c(Cl)c(Cl)cnn1Cc1ccccc1Cl] |
| C16.12 | 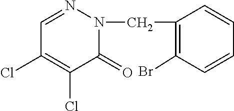 [SMILES: O=c1c(Cl)c(Cl)cnn1Cc1ccccc1Br] |
| C16.23 | 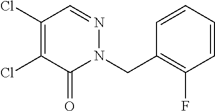 [SMILES: O=c1c(Cl)c(Cl)cnn1Cc1ccccc1F] |
| C16.24 | 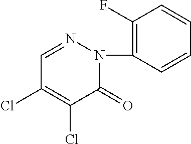 [SMILES: O=c1c(Cl)c(Cl)cnn1-c1ccccc1F] |
| C16.20 | 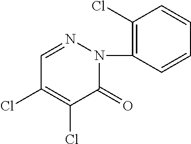 [SMILES: O=c1c(Cl)c(Cl)cnn1-c1ccccc1Cl] |
| C16.2 | 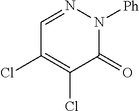 [SMILES: O=c1c(Cl)c(Cl)cnn1-c1ccccc1] |
| C16 | 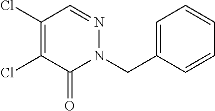 [SMILES: O=c1c(Cl)c(Cl)cnn1Cc1ccccc1] |
| C16.4 | 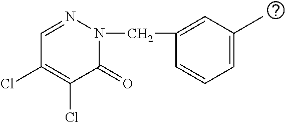 [SMILES: Cc1cccc(Cn2ncc(Cl)c(Cl)c2=O)c1] |
| C16.13 | 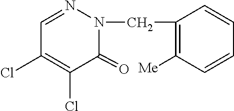 [SMILES: Cc1ccccc1Cn1ncc(Cl)c(Cl)c1=O] |
| C16.16 | 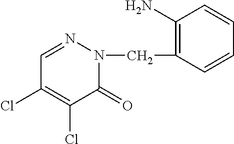 [SMILES: Nc1ccccc1Cn1ncc(Cl)c(Cl)c1=O] |
| 15 | 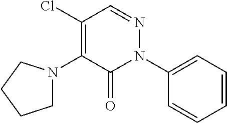 [SMILES: O=c1c(N2CCCC2)c(Cl)cnn1-c1ccccc1] |
| 14 | 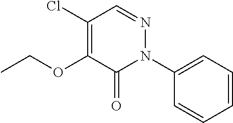 [SMILES: CCOc1c(Cl)cnn(-c2ccccc2)c1=O] |
| 16.14 | 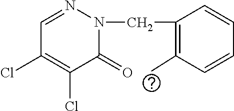 [SMILES: Cc1ccccc1Cn1ncc(Cl)c(Cl)c1=O] |
| 13 | 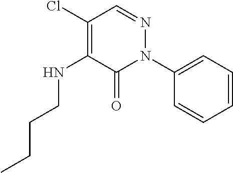 [SMILES: CCCCNc1c(Cl)cnn(-c2ccccc2)c1=O] |
| 16.22 | 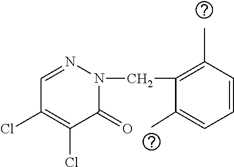 [SMILES: Cc1cccc(C)c1Cn1ncc(Cl)c(Cl)c1=O] |
| 12 | 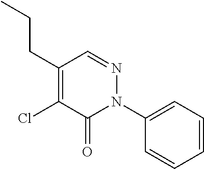 [SMILES: CCCc1cnn(-c2ccccc2)c(=O)c1Cl] |
| 16.8 | 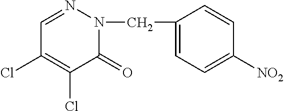 [SMILES: O=c1c(Cl)c(Cl)cnn1Cc1ccc([N+](=O)[O-])cc1] |
| 17 | 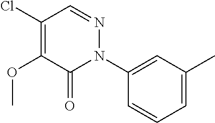 [SMILES: COc1c(Cl)cnn(-c2cccc(C)c2)c1=O] |
| 16.7 | 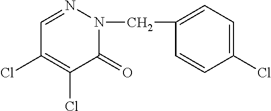 [SMILES: O=c1c(Cl)c(Cl)cnn1Cc1ccc(Cl)cc1] |
| 16.17 | 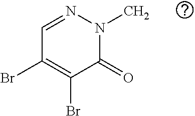 |
| 16.11 | 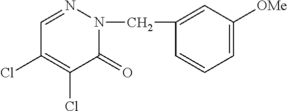 [SMILES: COc1cccc(Cn2ncc(Cl)c(Cl)c2=O)c1] |
| 16.9 | 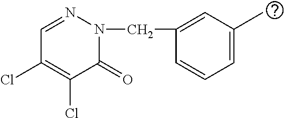 [SMILES: Cc1cccc(Cn2ncc(Cl)c(Cl)c2=O)c1] |
| 16.21 | 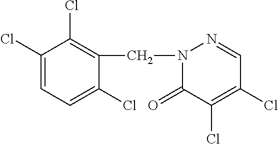 [SMILES: O=c1c(Cl)c(Cl)cnn1Cc1c(Cl)ccc(Cl)c1Cl] |
| 16.1 | 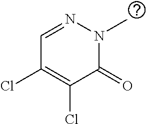 |
| 16.3 | 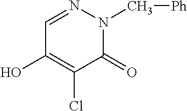 [SMILES: O=c1c(Cl)c(O)cnn1Cc1ccccc1] |
| 16.5 | 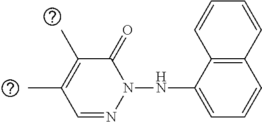 [SMILES: Cc1cnn(Nc2cccc3ccccc23)c(=O)c1C] |
| 16.10 | 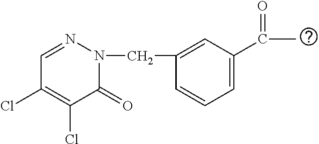 [SMILES: CC(=O)c1cccc(Cn2ncc(Cl)c(Cl)c2=O)c1] |
| 16.15 | 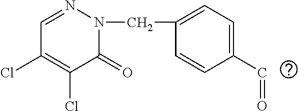 [SMILES: O=Cc1ccc(Cn2ncc(Cl)c(Cl)c2=O)cc1] |
| 16.27 | 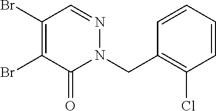 [SMILES: O=c1c(Br)c(Br)cnn1Cc1ccccc1Cl] |
| 16.43 | 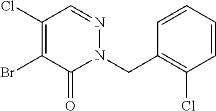 [SMILES: O=c1c(Br)c(Cl)cnn1Cc1ccccc1Cl] |
| 16.44 | 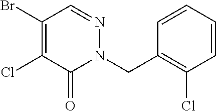 [SMILES: O=c1c(Cl)c(Br)cnn1Cc1ccccc1Cl] |
| 16.42 | 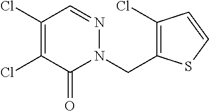 [SMILES: O=c1c(Cl)c(Cl)cnn1Cc1sccc1Cl] |
| 16.35 | 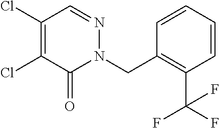 [SMILES: O=c1c(Cl)c(Cl)cnn1Cc1ccccc1C(F)(F)F] |
| 16.36 | 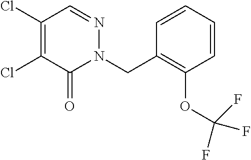 [SMILES: O=c1c(Cl)c(Cl)cnn1Cc1ccccc1OC(F)(F)F] |
| 16.25 | 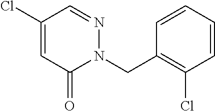 [SMILES: O=c1cc(Cl)cnn1Cc1ccccc1Cl] |
| 16.40 | 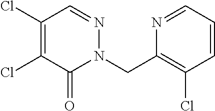 [SMILES: O=c1c(Cl)c(Cl)cnn1Cc1ncccc1Cl] |
| 16.39 | 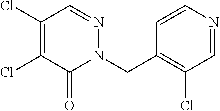 [SMILES: O=c1c(Cl)c(Cl)cnn1Cc1ccncc1Cl] |
| 16.26 | 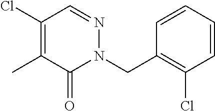 [SMILES: Cc1c(Cl)cnn(Cc2ccccc2Cl)c1=O] |
| 16.41 | 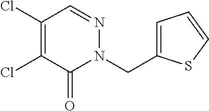 [SMILES: O=c1c(Cl)c(Cl)cnn1Cc1cccs1] |
| 16.37 | 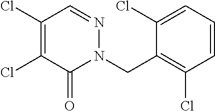 [SMILES: O=c1c(Cl)c(Cl)cnn1Cc1c(Cl)cccc1Cl] |
| X1 | 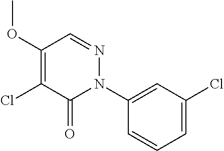 [SMILES: COc1cnn(-c2cccc(Cl)c2)c(=O)c1Cl] |
| X2 | 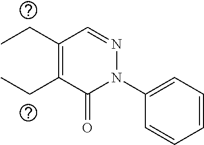 [SMILES: CCc1cnn(-c2ccccc2)c(=O)c1CC] |
| X3 | 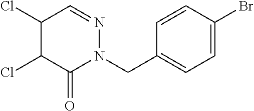 [SMILES: O=C1C(Cl)C(Cl)C=NN1Cc1ccc(Br)cc1] |
| X4 | 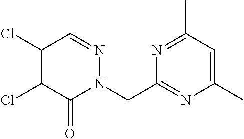 [SMILES: Cc1cc(C)nc(CN2N=CC(Cl)C(Cl)C2=O)n1] |
| X5 | 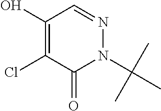 |
| X6 | 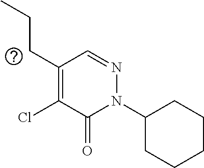 [SMILES: CCCc1cnn(C2CCCCC2)c(=O)c1Cl] |

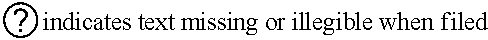

In one embodiment, the compound inhibits DsbB of one or more bacteria in an assay such as that described herein (e.g., in vitro or in vivo). One useful method for determining IC50 of the compounds of the instant invention is the in vitro E. coli assay with strain DHB7935 described in the Examples section herein. In one embodiment, the compound inhibits the DsbB in such an assay with an IC 50 of ≦50 μM. In one embodiment, the compound inhibits DsbB with an IC 50 of ≦25 μM. In one embodiment, the compound inhibits DsbB with an IC 50 of ≦12 μM. In one embodiment, the compound inhibits the DsbB with an IC 50 of ≦9 μM. In one embodiment, the compound inhibits DsbB with an IC 50 of ≦8.5 μM. In one embodiment, the compound inhibits DsbB with an IC 50 of ≦6 μM. In one embodiment, the compound inhibits DsbB with an IC 50 between 6 μM and 3 μM. In one embodiment, the compound inhibits DsbB with an IC 50 of ≦3 μM. In one embodiment, the compound inhibits DsbB with an IC 50 between 3 μM and 0.5 μM (e.g., ≦2 μM, ≦1 μM). In one embodiment, the compound inhibits DsbB with an IC 50 of ≦0.5 μM. In one embodiment, the compound inhibits DsbB with an IC 50 between 0.5 μM and 0.01 μM (e.g., 0.5 μM, ≦0.4 μM, ≦0.3 μM, ≦0.2 μM, 50.1 μM, ≦0.09 μM, ≦0.08 μM, ≦0.07 μM, ≦0.06 μM, ≦0.05 μM, ≦0.04 μM, ≦0.03 μM, ≦0.02 μM, ≦0.01 μM). In one embodiment, the compound inhibits the DsbB with an IC 50 of ≦0.5 μM. The in vitro E. coli DHB7935 assay described herein is a useful assay with which to characterize the compounds of the instant invention. The skilled artisan will recognize that a similar assay performed utilizing a naturally occurring bacteria (e.g., a pathogen) would be expected to indicate a substantially higher IC50 than the weaker expressing E. coli DHB7935 strain.

Another useful assay to characterize the compound of the instant invention is the Relative Inhibitory Concentration 50 (RIC50) assay described in the Examples section herein. In one embodiment, a compound of the instant invention is expected to have a RIC50 of 100 μM. Stronger inhibitors have been obtained, and in one embodiment, a compound of the instant invention has a RIC50 between 75 μM and 10 μM (e.g., of ≦75 μM, ≦50 μM, ≦25 μM, ≦20 μM, ≦≦15 μM, ≦10 μM). In one embodiment, a compound of the instant invention has a RIC50 between 10 μM and 1 μM (e.g., of ≦10 μM, ≦9 μM, ≦8 μM, ≦7 μM, ≦6 μM, ≦≦5 μM, ≦4 μM, ≦3 μM, ≦2 μM). In one embodiment, a compound of the instant invention has a RIC50 between 1 μM and 0.1 μM (e.g., of ≦1 μM, ≦0.9 μM, ≦0.8 μM, ≦0.7 μM, ≦0.6 μM, ≦0.5 μM, ≦0.4 μM, ≦0.3 μM, ≦0.2 μM). In one embodiment, a compound of the instant invention has a RIC50≦0.1 μM (e.g., ≦0.09 μM, ≦0.08 μM, ≦0.07 μM, ≦0.06 μM, ≦0.05 μM, ≦0.04 μM, ≦0.03 μM, ≦0.02 μM, ≦0.01 μM).

In one embodiment, the compound is in the form of a pharmaceutical composition. Such a pharmaceutical composition is typically formulated for use (externally or internally) with a potential multicellular host of a bacteria (e.g., a subject as described herein). In one embodiment the composition is an antibacterial composition. As the term is used herein an antibacterial composition contains the compound described herein that inhibits the activity of DsbB from one or more bacteria by the in vitro E. coli assay with strain DHB7935 described here, with a preferred IC 50 of ≦50 μM, ≦25 μM, ≦12 μM, ≦9 μM, ≦8 μM, ≦6 μM, ≦3 μM, ≦2 μM, ≦1 μM, ≦0.5 μM, ≦0.4 μM, ≦0.3 μM, ≦0.2 μM, ≦0.1 μM, ≦0.09 μM, ≦0.08 μM, ≦0.07 μM, ≦0.06 μM, ≦0.05 μM, ≦0.04 μM, ≦0.03 μM, ≦0.02 μM, ≦0.01 μM. In one embodiment, the antibacterial composition comprising the compound described herein inhibits the bacteria upon contact. In one embodiment, the compound is one of the compounds listed in Table 1.

In one embodiment the antibacterial composition is not intended for use with a potential multicellular host of a bacteria (e.g., a subject as described herein). Such a composition can be intended for use on a solid or semisolid surface, e.g., for decontamination, and formulated for such use. In one embodiment, the composition is formulated or use in vitro, for example, for use in cell culture or tissue culture in the laboratory, to prevent or inhibit contamination of the culture.

Various compounds described herein (e.g., the compounds of Formula I, II, and III, the compounds listed in Table 1) have been shown to inhibit DsbB in a DsbB expressing bacteria. As such, one aspect of the invention relates to a method of inhibiting DsbB in a bacteria that expresses DsbB, by contacting the bacteria with an effective amount of a composition comprising one or more of the compounds described herein. An effective amount would be an amount to deliver a concentration sufficient to inhibit a substantial amount of DsbB activity in the contacted microbe. Such an amount can be determined by standard assays, some of which are described herein. Such method may be performed in vivo or in vitro, as described herein. Inhibition of DsbB in a bacteria can affect the growth of the bacteria, and can affect the virulence of the bacteria.

One aspect of the invention relates to a method of inhibiting a bacteria by contacting an effective amount of the composition to the bacteria.

One aspect of the invention relates to a method of treating a bacterial infection in a subject in need thereof; by providing a composition comprising a compound of Formula I and administering a therapeutically effective amount of the composition to the subject to thereby contact the bacteria with an effective amount of the compound, and thereby treat the bacterial infection.

Aspects of the present invention relate to methods of using a composition comprising a compound identified herein (e.g., Formula I, II, and III), herein referred to as “the composition”. Methods of using these compositions include administering the composition as a pharmaceutical composition to a subject (e.g. a subject in need of treatment a bacterial infection). In one embodiment the composition may be used for treating a bacterial infection or disease condition caused by or related to bacterial infection. The method comprises providing the composition and administering a therapeutically effective amount of the composition to the subject in need thereof. In one embodiment, the subject has a bacterial infection.

One aspect of the present invention relates to a method of inhibiting growth of a bacteria by contacting the bacteria with an effective amount of the composition.

Without being bound by theory, it is thought that one activity of the compounds described herein is to increase porosity of the bacterial membrane. As such, the compound can facilitate transport/delivery of molecules into the bacteria. This activity is thought to at least in part to promote synergy with a second agent. As such, one aspect of the present invention relates to a method of sensitizing a bacteria to inhibition (e.g. growth inhibition) by contacting the bacteria with an effective amount of a composition. Such sensitization can be in preparation for second contacting of the bacteria with a second agent to which the bacteria have been sensitized. The second agent can be contacted in an amount that is known to be effective in the absence of sensitization, or can be contacted in a reduced amount such as an amount that is effective with sensitization. In one embodiment, the contacting occurs in vivo. As such, a therapeutically effective amount of the composition formulated as a pharmaceutical composition is administered to a subject. The composition may comprise the second agent, or the second agent may be administered to the subject separately.

The herein described activities of the identified compounds indicates that their use with other known bacterial inhibitors (e.g., antibiotics) herein referred to as a second agent, can reduce the amount of the second agent needed to effectively inhibit the bacteria Using less of an agent reduces the likelihood of bacteria developing resistance to the agent. As such, one aspect of the present invention relates to a method of inhibiting the development of resistance by bacteria to such a second agent (e.g., an antibiotic) by a bacteria comprising, contacting the bacteria with an effective amount of the composition and a reduced amount of the second agent (e.g., antibiotic). A reduced amount, as the term is used herein, refers to an amount that is less than the typical prescribed dosage.

Aspects of the invention relate to specific formulations of the compound for use in the specific methods of use of such a composition. In one embodiment, the specific formulation is a pharmaceutical composition. The specific pharmaceutical composition will depend upon the route of administration, for example, a formulation for topical administration to a wound, or a formulation for parenteral administration. Such formulations are known in the art. The appropriate formulation is to be determined by the skilled practitioner for a given pathogen, infection and route of administration. Formulations described herein are also envisioned to contain a second agent that is potentiated by the compounds described herein, such as a bacterial inhibitor (e.g., an antibiotic). Examples of other such agents are provided herein.

Methods of using the composition involve contacting the composition to a bacteria in an effective amount to inhibit the bacteria. In one embodiment, the bacteria is within the body of a subject or patient. As such, the composition is in the form of a pharmaceutical composition which is administered to the subject by a route and in an amount sufficient to thereby contact an effective amount of that composition to the bacteria. In one embodiment, the subject is diagnosed with or suspected of having an infection with the bacteria. In this respect, the invention relates to a method of treating a bacterial infection or disease condition caused by or related to bacterial infection. In one embodiment, the subject is at risk for infection, but may not yet have developed an infection. For example, the subject may have been exposed to a specific bacterial pathogen, or may have a condition that puts them at risk for such an infection. In such circumstances, the composition is administered prophylactically to the subject. The method and dose of administration will depend upon the type of infection and specific bacteria. For example, a systemic infection such as sepsis may call for systemic administration. A localized infection (such as a topical infection) may only require localized administration such as to an infected wound.

Without being bound by theory, the compounds described herein are thought to inhibit the formation of disulfide bonds in molecules necessary for virulence of some bacteria by inhibiting the DsbB in the bacteria. Alternatively, or in addition, the compounds may inhibit growth of the bacteria. In some bacteria, the growth inhibition may only occur under specific conditions, (e.g. anaerobic growth conditions).

The contacting of the agent to the bacteria can occur in vivo or in vitro. Contacting in vitro can be, for example, in culture of the bacteria, or can be in a culture of cells or organism in which the bacteria is not desired (e.g., mammalian cell culture). Such contacting can be performed by including the agent in the media in which the cells, organism or tissue is grown.

Contacting in vivo is generally achieved by administration of the agent to a subject which is suspected of being infected by the bacteria. One of skill in the art will recognize that an effective amount for in vivo contact may require a higher dose of administration to result in a sufficient amount of target reaching the bacteria within the subject's body.

In one embodiment, the bacteria is contacted with the compound Formula I of the composition at a concentration of from about 25 μM to about 500 μM. In one embodiment, the concentration at which the compound is contacted to the bacteria is ≦500 μM, ≦450 μM, ≦400 μM, ≦350 μM, ≦300 μM, ≦250 μM, ≦200 μM, ≦150 μM, ≦100 μM, ≦50 μM, ≦40 μM, ≦30 μM.

Bacteria suitable for inhibition with the compositions of the present invention include, without limitation, the bacteria listed in Tables 2 and 3 below.

In one embodiment of the various compositions and methods described herein, one or more of the compounds specified in Table 1 and/or Table 9 and/or Table 10 is specifically excluded as the compound. For example, in one embodiment of the various compositions and methods described herein, the compound is not 16.27. In one embodiment of the various compositions and methods described herein, one or more of the following molecules listed in Table 1 and/or Table 9 (1, 4, 8, 23, 18, 16.6, 16.12, 16.20, 16.2, 16.23, 16.13, 16, 16.14, 16.17, 16.24, 16.4, 16.22, 14, 15, 13, 16.8, 12, 17, 16, 16, 16.11, 16.9, 16.7, 16.21, 16.1, 16.3, 16.5, 16.10, 16.15, 16.18, 16.19, 16.25, 16.26, 16.28, 16.29, 16.30, 16.31, 16.32, 16.33, 16.34, 16.35, 16.36, 16.37, 16.38, 16.39, 16.40, 16.41, 16.42, 16.43, or 16.44) is specifically excluded as the compound.

In one embodiment of the various compositions and methods described herein, the compound is 16.25, 16.26, 16.27, 16.28, 16.29, 16.30, 16.31, 16.32, 16.33, 16.34, 16.35, 16.36, 16.37, 16.38, 16.39, 16.40, 16.41, 16.42, 16.43, or 16.44. In one embodiment of the various compositions and methods described herein, the compound is 16.25, 16.26, 16.28, 16.29, 16.30, 16.31, 16.32, 16.33, 16.34, 16.35, 16.36, 16.37, 16.38, 16.39, 16.40, 16.41, 16.42, 16.43, or 16.44.

TABLE 2
Critical bacterial virulence factors that are DsbA substrates, and relevant bacteria.

| | | |
|---|---|---|
| Organism | DabA substrate | Substrate function |

Adhesion

| | | |
|---|---|---|
| Uropathogenic Escherichia coli | PapD | Molecular chaperone of P fimbriae |
| Enteropathogenic E. coli | BfpA | Major structural subunit of bundle-forming pill |
| Salmonella enterica | PefA | Major structural subunit of plasmid-encoded |
| | | fimbriae |

Toxin production and secretion

| | | |
|---|---|---|
| Enterotoxigenic E. coli | STₛ | Heat-stable enterotoxin |
| Enterotoxigenic E. coli | LT | Heat-labile enterotoxin |
| Bordetella pertussis | S1 and S2 | Pertussis toxin A and B subunits |
| Vibrio cholerae | Unknown | Role in secretion of cholera toxin A subunit? |

Secreted enzymes and secretion components

| | | |
|---|---|---|
| Klebsiella oxytoca | PulA | Pullulanase |
| K. oxytoca | PulS and Pulk | Components of the type II secretion system |
| Erwinia chrysanthemi | EGZ, PelB and PelC | Cellulase and pectate lyases |
| Erwinia carotovora subsp. | PelC and Peh | Pectate lyase and endopolygalacturonase |
| carotovora | | |
| E. carotovora subsp. atroseptica | PelA-C, CelV, PrtW, Svx, Nip, | Secreted enzymes |
| | ECA0852, PehA and PehX | |
| Pseudomonas aeruginosa | LasB | Elastase |
| Haemophilus influenzae | HbpA | Haem transport protein |

It is envisioned that gram (−) bacteria will be affected by the compounds identified herein. However, in some circumstances, gram (+) bacteria may be affected as well. Pathogenic bacteria are envisioned as a target of the methods described herein. The compositions described herein can also be used to inhibit non-pathogenic bacteria as well. Examples of bacteria for inhibition with the herein disclosed compounds include, without limitation, Salmonella typhimurium, Klebsiella pneumoniae, Vibrio cholera, Haemophilus influenza, Francisella tularensis, Klebsiella oxytoca, Enterobacter cloacae, Enterobacter aerogenes, Citrobacter freundii, Pseudomonas aeruginosa, Acinetobacter baumannii, Helicobacter pylori, and combinations thereof

TABLE 3
DsbBs from pathogenic gram-negative bacteria that can complement
ΔdsbB E. coli.

| | | | Protein |
|---|---|---|---|
| Organism | Name | % identity | length |
| Escherichia coli | ECdsbB | 100 | 177 |
| Salmonella typhimurium LT2 | STdsbB | 85 | 177 |
| Salmonella typhimurium LT2 | STdsbI | 21 | 226 |
| Klebsiella pneumoniae | KPdsbB | 79 | 177 |
| W63917 | | | |
| Vibrio cholerae N16961 | VCdsbB | 47 | 174 |
| Haemophilus influenzae | HIdsbB | 41 | 178 |
| Pseudomonas aeruginosa | PΔdsbB | 27 | 170 |
| PA14 | | | |
| Pseudomonas aeruginosa | PAdsbH | 23 | 164 |
| PA14 | | | |
| Acinetobacter baumannii A1 | ABdsbB | 20 | 172 |
| Francisella tularensis LVS | FTdsbB | 11 | 164 |

### Pharmaceutical Compositions

Another aspect of the invention relates to a pharmaceutical composition including a pharmaceutically acceptable excipient along with a therapeutically effective amount of one or more compound(s) identified herein. “Pharmaceutically acceptable excipient” means an excipient that is useful in preparing a pharmaceutical composition that is generally safe, non-toxic, and desirable, and includes excipients that are acceptable for veterinary use as well as for human pharmaceutical use. Such excipients may be solid, liquid, semisolid, or, in the case of an aerosol composition, gaseous.

In one embodiment, the pharmaceutical composition may be formulated for delivery via a specific route of administration, examples of such routes are provided herein. “Route of administration” may refer to any administration pathway known in the art, including but not limited to aerosol, nasal, oral, transmucosal, transdermal, parenteral, enteral, or ocular. “Transdermal” administration may be accomplished using a topical cream or ointment or by means of a transdermal patch. “Parenteral” refers to a route of administration that is generally associated with injection, including intraorbital, infusion, intraarterial, intracapsular, intracardiac, intradermal, intramuscular, intraperitoneal, intrapulmonary, intraspinal, intrasternal, intrathecal, intrauterine, intravenous, subarachnoid, subcapsular, subcutaneous, transmucosal, or transtracheal. Via the parenteral route, the compositions may be in the form of solutions or suspensions for infusion or for injection, or as lyophilized powders. Via the enteral route, the pharmaceutical compositions can be in the form of tablets, gel capsules, sugar-coated tablets, syrups, suspensions, solutions, powders, granules, emulsions, microspheres or nanospheres or lipid vesicles or polymer vesicles allowing controlled release. Via the topical route, the pharmaceutical compositions based on compounds according to the invention may be formulated for treating the skin and mucous membranes and are in the form of ointments, creams, milks, salves, powders, impregnated pads, solutions, gels, sprays, lotions or suspensions. They can also be in the form of microspheres or nanospheres or lipid vesicles or polymer vesicles or polymer patches and hydrogels allowing controlled release. These topical-route compositions can be either in anhydrous form or in aqueous form depending on the clinical indication. Via the ocular route, they may be in the form of eye drops.

The pharmaceutical compositions according to the invention can also contain any pharmaceutically acceptable carrier. “Pharmaceutically acceptable carrier” as used herein refers to a pharmaceutically acceptable material, composition, or vehicle that is involved in carrying or transporting a compound of interest from one tissue, organ, or portion of the body to another tissue, organ, or portion of the body. For example, the carrier may be a liquid or solid filler, diluent, excipient, solvent, or encapsulating material, or a combination thereof. Each component of the carrier must be “pharmaceutically acceptable” in that it must be compatible with the other ingredients of the formulation. It must also be suitable for use in contact with any tissues or organs with which it may come in contact, meaning that it must not carry a risk of toxicity, irritation, allergic response, immunogenicity, or any other complication that excessively outweighs its therapeutic benefits.

The pharmaceutical compositions according to the invention can also be encapsulated, tableted or prepared in an emulsion or syrup for oral administration. Pharmaceutically acceptable solid or liquid carriers may be added to enhance or stabilize the composition, or to facilitate preparation of the composition. Liquid carriers include syrup, peanut oil, olive oil, glycerin, saline, alcohols and water. Solid carriers include starch, lactose, calcium sulfate, dihydrate, terra alba, magnesium stearate or stearic acid, talc, pectin, acacia, agar or gelatin. The carrier may also include a sustained release material such as glyceryl monostearate or glyceryl distearate, alone or with a wax.

The pharmaceutical preparations are made following the conventional techniques of pharmacy involving milling, mixing, granulation, and compressing, when necessary, for tablet forms; or milling, mixing and filling for hard gelatin capsule forms. When a liquid carrier is used, the preparation will be in the form of a syrup, elixir, emulsion or an aqueous or non-aqueous suspension. Such a liquid formulation may be administered directly or filled into a soft gelatin capsule.

### Administration

Administration is performed to promote contact of an effective amount of the administered compound and/or second agent to the microbe within the subject. A therapeutically effective amount of the compound and/or agent or pharmaceutical composition containing the compound and/or agent is administered to the subject. The method may further comprise selecting a subject in need of such treatment (e.g., identification of an infected subject. In one embodiment, the agent is administered in combination with or concurrently with one or more other agents that inhibit microbial growth (e.g., those described herein).

Methods of administration include systemic and localized (e.g., topical). Without limitation, these routes include, parenteral administration, and enteral administration.

The route of administration may be intravenous (I.V.), intramuscular (I.M.), subcutaneous (S.C.), intradermal (I.D.), intraperitoneal (I.P.), intrathecal (I.T.), intrapleural, intrauterine, rectal, vaginal, topical, and the like. The compounds of the invention can be administered parenterally by injection or by gradual infusion over time and can be delivered by peristaltic means. Administration may be by transmucosal or transdermal means. For transmucosal or transdermal administration, penetrants appropriate to the barrier to be permeated are used in the formulation. Such penetrants are generally known in the art, and include, for example, for transmucosal administration bile salts and fusidic acid derivatives. In addition, detergents may be used to facilitate permeation. Transmucosal administration may be through nasal sprays, for example, or using suppositories. For oral administration, the compounds of the invention are formulated into conventional oral administration forms such as capsules, tablets and tonics.

For topical administration, the pharmaceutical composition (inhibitor of kinase activity) is formulated into ointments, salves, gels, or creams, as is generally known in the art.

The therapeutic compositions of this invention are conventionally administered in the form of a unit dose. The term “unit dose” when used in reference to a therapeutic composition of the present invention refers to physically discrete units suitable as unitary dosage for the subject, each unit containing a predetermined quantity of active material calculated to produce the desired therapeutic effect in association with the required diluent; i.e., carrier, or vehicle.

The compositions are administered in a manner compatible with the dosage formulation, and in a therapeutically effective amount. The quantity to be administered and timing depends on the subject to be treated, capacity of the subject's system to utilize the active ingredient, and degree of therapeutic effect desired. The precise therapeutically effective amount is that amount of the composition that will yield the most effective results in terms of efficacy of treatment in a given subject. This amount will vary depending upon a variety of factors, including but not limited to the characteristics of the therapeutic compound (including activity, pharmacokinetics, pharnmacodynamics, and bioavailability), the physiological condition of the subject (including age, sex, disease type and stage, general physical condition, responsiveness to a given dosage, and type of medication), the nature of the pharmaceutically acceptable carrier or carriers in the formulation, and the route of administration. One skilled in the clinical and pharmacological arts will be able to determine a therapeutically effective amount through routine experimentation, for instance, by monitoring a subject's response to administration of a compound and adjusting the dosage accordingly. For additional guidance, see Remington: The Science and Practice of Pharmacy (Gennaro ed. 20th edition, Williams & Wilkins PA, USA) (2000).

In one embodiment, the term “therapeutically effective amount” refers to an amount that is sufficient to effect a therapeutically or prophylactically significant reduction in a symptom associated with an infection of a microbe when administered to a typical subject who has the infection. A therapeutically or prophylactically significant reduction in a symptom is, e.g. about 10%, about 20%, about 30%, about 40%, about 50%, about 60%, about 70%/a, about 80%, about 90%, about 100%, about 125%, about 150% or more as compared to a control or non-treated subject. In many instances, the specific therapeutically effective amount will depend upon many factors, such as the specific microbe and the overall condition of the subject, and will be determined by the skilled practitioner who takes all such relevant factors into consideration. An acceptable benefit/risk ratio will also be considered when determining a therapeutically effective amount. Such amounts will depend, of course, on the particular condition being treated, the severity of the condition and individual patient parameters including age, physical condition, size, weight and concurrent treatment. These factors are well known to those of ordinary skill in the art and can be addressed with no more than routine experimentation. It is preferred generally that a maximum dose be used, that is, the highest safe dose according to sound medical judgment. It will be understood by those of ordinary skill in the art, however, that a lower dose or tolerable dose can be administered for medical reasons, psychological reasons or for virtually any other reasons.

In addition, the amount of each component to be administered also depends upon the frequency of administration, such as whether administration is once a day, twice a day, 3 times a day or 4 times a day, once a week; or several times a week, for example 2 or 3, or 4 times a week.

### Dosage

Typical dosages of an effective amount of the composition of the invention can be in the ranges recommended by the manufacturer where known therapeutic compounds are used, and also as indicated to the skilled artisan by the in vitro responses or responses in animal models. Such dosages typically can be reduced by up to about one order of magnitude in concentration or amount without losing the relevant biological activity. Thus, the actual dosage will depend upon the judgment of the physician, the condition of the patient, and the effectiveness of the therapeutic method based, for example, on the in vitro responsiveness of the relevant primary cultured cells or histocultured tissue sample, or the responses observed in the appropriate animal models, as previously described.

### Combination Therapy

It is appreciated that the compounds and pharmaceutical compositions of the present invention can be formulated and employed in combination therapies, that is, the compounds and pharmaceutical compositions can be formulated with or administered concurrently with, prior to, or subsequent to, one or more other desired therapeutics (e.g., second agents as described herein) or medical procedures. The particular combination of therapies (therapeutics or procedures) to employ in a combination regimen will take into account compatibility of the desired therapeutics and/or procedures and the desired therapeutic effect to be achieved. It is appreciated that the therapies employed can achieve a desired effect for the same disorder (for example, an inventive composition can be administered concurrently with another antibiotic), or they can achieve different effects (e.g., control of an adverse effects).

For example, other agents that can be used in combination with the compounds of the present invention for treating a bacterial infection include an agent that can be an anti-infective agent or an antibiotic. The term “antibiotic” is used herein to describe a compound or composition which decreases the viability of a microorganism, or which inhibits the growth or reproduction of a microorganism. Exemplary antibiotics include, but are not limited to penicillins, cephalosporins, penems, carbapenems, monobactams, aminoglycosides, sulfonamides, macrolides, tetracyclins, lincosides, quinolones, chloramphenicol, vancomycin, metronidazole, rifampin, isoniazid, spectinomycin, trimethoprim, sulfamethoxazole, and the like. Other agents include, without limitation, anti-fouling or biocidal, bacteriostatic or bactericidal agents, or other antibacterial agents.

In one embodiment, the pharmaceutical composition further comprises one or more additional therapeutically active ingredients (e.g., antibiotic or a palliative agent). For purposes of the invention, the term “palliative” refer, to treatment that is focused on the relief of symptoms of a disease and/or side effects of a therapeutic regimen, but is not curative.

### Kits

The present invention is also directed to a kit to treat a bacterial infection. The kit is an assemblage of materials or components, including at least one of the compounds identified herein formulated as a composition or therapeutic composition as described above. Thus, in one embodiment the kit contains a tool for the administration of the compositions contained therein. In one embodiment, the kit contains a second agent for use in conjunction with the compositions contained therein.

In one embodiment, the kit is configured particularly for the purpose of treating mammalian subjects. In another embodiment, the kit is configured particularly for the purpose of treating human subjects. In further embodiments, the kit is configured for veterinary applications, treating subjects such as, but not limited to, farm animals, domestic animals, and laboratory animals.

In one embodiment the kit contains instructions regarding the dosage of the compositions and any second agent contained therein. Instructions for use may be included in the kit. “Instructions for use” typically include a tangible expression describing the technique to be employed in using the components of the kit to effect a desired outcome. Optionally, the kit also contains other useful components, such as, diluents, buffers, pharmaceutically acceptable carriers, syringes, catheters, applicators, pipetting or measuring tools, or other useful paraphernalia as will be readily recognized by those of skill in the art.

The materials or components assembled in the kit can be provided to the practitioner stored in any convenient and suitable ways that preserve their operability and utility. For example the components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit, such as inventive compositions and the like. The packaging material is constructed by well-known methods, preferably to provide a sterile, contaminant-free environment. As used herein, the term “package” refers to a suitable material such as glass, plastic, paper, foil, and the like, capable of holding the individual kit components. Thus, for example, a package can be a glass vial used to contain suitable quantities of an inventive composition. The packaging material generally has an external label which indicates the contents and/or purpose of the kit and/or its components.

### Cleaning Compositions

One aspect of the invention relates to a cleaning composition comprising a compound identified herein. In one embodiment, the cleaning composition comprises nanoparticles. In one embodiment, the cleaning composition is used for cleaning and protecting surfaces with all the advantages of the prior art and the additional benefit of having the added effect of the activity of the compounds (inhibiting bacteria, potentiating activity of a second agent). In one embodiment, the cleaning composition has antibacterial activity.

The cleaning composition can be in a variety of forms (e.g., liquid, aqueous solution, solid, powder, foam, gel). In one embodiment, the cleaning composition is embedded in a support e.g., styrofoam). In one embodiment, the cleaning solution is a time release system. In one embodiment, the cleaning solution is a concentrate that requires dilution before use.

A cleaning composition of the invention can be used to clean any type of surface, including but not limited to plastic, leather, vinyl, tiles, ceramic, marble, granite, stainless steel, paper, acrylic resin, food packaging, and composite materials. Additional examples of surfaces that can be clean using a cleaning composition of the invention include but are not limited to flooring, appliances, such as but not limited to kitchen appliances and cookware, medical and surgical apparatus and devices, cosmetic apparatus and devices such as comb, brushes, and sponges, textiles such as medical and surgical gowns and sheets, disposable and non-disposable diapers and wipes, camping gear, furniture, such as but not limited to bed and spring boxes, bathrooms, carpets, rugs.

### Coatings of Substrates

Another aspect of the invention relates to the composition coated onto a solid or semisolid matrix or substrate. Such formulations of the compositions, as well as such coated or impregnated substrates are encompassed by the invention. In one embodiment, the formulation is a gel coating specifically formulated for slow release of the composition into a surrounding aqueous environment.

In one embodiment, the substrate or matrix is in the form of an indwelling device. In another embodiment, the compound is formulated with a coating agent to adhere to a biomaterial surface, such as teeth, bone, skin, etc. In one embodiment, the coating agent is formulated to adhere to or be absorbed by a fabric, cloth or membrane, such as a bandage or other wound dressing. Another example of a membrane is a water treatment membrane. In one embodiment, the carrier is formulated for inclusion into a product for application to a body surface, such as personal care product.

In one embodiment, the compound is formulated to adhere to a device that is to contact a living medium (the medium around or within a multicellular organism). For example, to be delivered, contacted into, or otherwise implanted, into a living multicellular organism. Such devices are sometimes referred to in the art as indwelling devices. Examples of such devices include, without limitation, catheters, surgical implants, prosthetic devices, surgery tools, endoscopes, contact lenses, etc.

### Screening Assays

Aspects of the invention relate to a screening assay for the identification of additional compounds with the activity (e.g., antibacterial, growth inhibitory, anti-virulence) as the compounds identified herein. Such compounds specifically inhibit DsbB in a bacteria. Working examples of such assays are provided herein. In one embodiment, the assay method comprises testing one or more test agents in a β-gal disulfide bond formation assay. The assay uses β-gal fused to a bacterial membrane protein, and in the assays DsbB functions as the oxidant of DsbA. In the assay, test agents that significantly inhibit disulfide bond formation are identified, and then further tested in a second (control) β-gal disulfide bond formation assay, which uses β-gal fused to a bacterial membrane protein, where bVKOR functions as the oxidant of DsbA in the assay. The ability of the test agent(s) to significantly inhibit disulfide bond formation in the first assay, and the inability of the test agent(s) to inhibit disulfide bond formation in the second assay indicates that the test agent(s) specifically inhibits DsbB.

Another aspect of the invention relates to a screening assay for identifying an agent that specifically inhibits bVKOR. Such agents are useful in inhibiting bacteria which naturally express bVKOR, such as M. tuberculosis. In the method, one or more test agents is tested in a β-gal disulfide bond formation assay. The assay uses β-gal fused to a bacterial membrane protein, and bVKOR functions as the oxidant of DsbA. Test agents identified as significantly inhibiting disulfide bond formation in this first assay are then subjected to a second (control) assay in which they are further tested in a β-gal disulfide bond formation assay using β-gal fused to a bacterial membrane protein, wherein DsbB functions as the oxidant of DsbA in the assay. The ability of the test agent to significantly inhibit disulfide bond formation in the first assay and the inability of the test agent to inhibit disulfide bond formation in the second assay indicates that the test agent specifically inhibits bVKOR.

The assay can be performed in a number of different bacteria. In one embodiment, the bacteria used for the assay is E. coli. In one embodiment, the bacterial membrane protein used if MalF to produce a MalF-β-Gal fusion protein. In one embodiment, the MalF-β-Gal fusion protein is as per Froshauer et al., (J Mol Biol. 200: 501-11 (1988); Bardwell, J. C. A., McGovern, K., and Beckwith, J. Identification of a protein required for disulfide bond formation in vivo. Cell. 67:581-589 (1991)). Specific methods for constructing and performing the assays can be adapted from U.S. Patent Publication 2011/0243958 (Oct. 6, 2011), the contents of which are incorporated herein by reference in their entirety.

In one embodiment, the assay is a high throughput assay performed by a non-human machine. In one embodiment, the assay is performed as a color assay with bacteria grown on agar that comprise X-gal (5-bromo-4-chloro-3-indolyl-β-D-galactopyranoside, BCIG) and the color readout is performed by a non-human machine.

A variety of different species of DsbB and of different species of bVKOR are known in the art and can be used in the assays described herein. Examples of microbes that contain DsbB are shown in Table 3. The amino acid sequence of the VKOR of Mycobacterium tuberculosis is known in the art, for example, is provided in US Patent Publication 2011/0243958, the contents of which are herein incorporated by reference in their entirety. Other microbes known to contain VKOR include, without limitation those listed in Table 4 below.

TABLE 4
Microbes that contain VKOR

Actinobacteria
Rubrobacteridae
Rubrobacter xylanophilus (strain DSM 9941/NBRC 16129)
Symbiobacterium thermophilum
Actinobacteridae (
Streptomyces tenjimariensis
Streptomyces coelicolor
Streptomyces avermitilis
Corynebacterium glutamicum (Brevibacterium flavum)
Corynebacterium efficiens
Corynebacterium jeikeium (strain K411)
Mycobacterium ulcerans (strain Agy99)
Mycobacterium sp (strain MCS)
Mycobacterium sp JLS
Mycobacterium flavescens PYR-GCK
Mycobacterium sp KMS
Mycobacterium leprae
Mycobacterium
Mycobacterium tuberculosis
Mycobacterium bovis
Mycobacterium paratuberculosis
Mycobacterium avium (strain 104)
Mycobacterium bovis (strain BCG/Paris 1173P2)
Mycobacterium tuberculosis (strain F11)
Mycobacterium vanbaalenii (strain DSM 7251/PYR-1)
Mycobacterium smegmatis
Mycobacterium smegmatis (strain ATCC 700084/mc(2)155)
Rhodococcus sp (strain RHA1)
Nocardia farcinica
Rhodococcus erythropolis
Rhodococcus erythropolis (strain PR4)
Gordonia westfalica
Nocardioides sp JS614
Arthrobacter aurescens (strain TC1)
Arthrobacter aurescens
Arthrobacter sp (strain FB24)
Tropheryma whipplei (strain Twist) (Whipple's bacillus)
Tropheryma whipplei (strain TW08/27) (Whipple's bacillus)
Microbacterium arborescens
Leifsonia xyli subsp xyli
Salinispora arenicola CNS205
Salinispora tropica CNB-440
Acidothermus cellulolyticus (strain ATCC 43068/11B)
Kineococcus radiotolerans SRS30216
Bifidobacterium longum
Bifidobacterium adolescentis (strain ATCC 15703/DSM 20083)
Proteobacteria
Sinorhizobium/Ensifergroup
Rhizobium meliloti (Sinorhizobium meliloti)
Sinorhizobium medicae WSM419
Sphingopyxis alaskensis (Sphingomonas alaskensis)
Oceanicola granulosus HTCC2516
Saccharophagus degradans (strain 2-40/ATCC 43961/DSM 17024)
Hahella chejuensis (strain KCTC 2396)
Azoarcus sp (strain EbN1)
Thiobacillus denitrificans (strain ATCC 25259)
Stigmatella aurantiaca DW4/3-1
Anaeromyxobacter sp Fw109-5
Myxococcus xanthus (strain DK 1622)
Anaeromyxobacter dehalogenans (strain 2CP-C)
Candidatus Desulfococcus oleovorans Hxd3
Bdellovibrio bacteriovorus
Bacteroidetes
Salinibacter ruber (strain DSM 13855)
Microscilla marina ATCC 23134
Flavobacterium johnsoniae UW101
Gramella forsetii (strain KT0803)
Planctomycetes
Planctomycetacia
Rhodopirellula baltica
Spirochaetes
Leptospira borgpetersenii serovar Hardjo-bovis (strain JB197)
Leptospira borgpetersenii serovar Hardjo-bovis (strain L550)
Leptospira interrogans serogroup Icterohaemorrhagiae serovar
copenhageni
Leptospira interrogans
Chloroflexi
Chloroflexus aurantiacus J-10-fl
Roseiflexus sp RS-1
Chloroflexus aggregans DSM 9485
Herpetosiphon aurantiacus ATCC 23779
Cyanobacteria
Trichodesmium erythraeum (strain IMS101)
Lyngbya sp PCC 8106
Gloeobacter violaceus
Prochlorococcus marinus
Prochlorococcus marinus str MIT 9303
Prochlorococcus marinus (strain MIT 9313)
Prochlorococcus marinus str NATL1A
Prochlorococcus marinus (strain MIT 9312)
Prochlorococcus marinus str MIT 9515
Prochlorococcus marinus subsp pastoris (strain CCMP 1378/MED4)
Prochlorococcus marinus str AS9601
Prochlorococcus marinus (strain NATL2A)
Anabaena sp (strain PCC 7120)
Nodularia spumigena CCY9414
Anabaena variabilis (strain ATCC 29413/PCC 7937)
Synechococcus sp (strain WH8102)
Synechococcus sp (strain ATCC 27144/PCC 6301/SAUG 1402/1)
(Anacystis nidulans)
Synechococcus sp (strain PCC 7942) (Anacystis nidulans R2)
Synechocystis sp (strain PCC 6803)
Synechococcus sp RS9916
Crocosphaera watsonii
Synechococcus sp (strain JA-3-3Ab) (Cyanobacteria bacterium
Yellowstone A-Prime)
Synechococcus sp (strain CC9311)
Synechococcus sp (strain CC9605)
Synechococcus elongatus (ThermoSynechococcus elongatus)
Synechococcus sp BL107
Synechococcus sp (strain CC9902)
Synechococcus sp (strain JA-2-3B'a(2-13)) (Cyanobacteria bacterium
Yellowstone B-Prime)
Chlamydiae
Protochlamydia amoebophila (strain UWE25)
Acidobacteria
Acidobacteria bacterium (strain E11in345)
Archaea
Metallosphaera sedula DSM 5348
Sulfolobus solfataricus
Sulfolobus acidocaldarius
Aeropyrum pernix
Pyrobaculum islandicum DSM 4184
Pyrobaculum aerophilum
Natronomonas pharaonis (strain DSM 2160/ATCC 35678)

### Test Agents

The screening assays described herein can be used to screen test agents for the ability to specifically inhibit bVKOR or DsbB. Test agents such as chemicals; small molecules; nucleic acid sequences (e.g., RNAi); nucleic acid analogues; proteins; peptides; aptamers; antibodies; or fragments thereof; can be identified or generated for use in the present invention to inhibit the expression or activity of bVKOR or DsbB.

Test agents in the form of a protein and/or peptide or fragment thereof can also be designed or identified to inhibit bVKOR or DsbB. Such agents encompass proteins which are normally absent or proteins that are normally endogenously expressed in mammals (e.g. human). Examples of useful proteins are mutated proteins or otherwise modified proteins, fragments of proteins, genetically engineered proteins, genetically modified proteins, peptides, synthetic peptides, recombinant proteins, chimeric proteins, antibodies, midibodies, minibodies, triabodies, humanized proteins, humanized antibodies, chimeric antibodies, modified proteins and fragments thereof. In one embodiment, the agent is a ligand or a portion thereof; or a modified ligand or modified portion thereof. Agents also include antibodies (polyclonal or monoclonal), neutralizing antibodies, antibody fragments, peptides, proteins, peptide-mimetics, aptamers, oligonucleotides, hormones, small molecules, nucleic acids, nucleic acid analogues, carbohydrates or variants thereof that function to inactivate the nucleic acid and/or protein of the gene products identified herein, and those as yet unidentified.

In one embodiment, the agent is a known or unknown compound. It can be from one of numerous chemical classes, such as organic molecules, which may include organometallic molecules, inorganic molecules, genetic sequences, etc. Agents may also be fusion proteins from one or more proteins, chimeric proteins (for example domain switching or homologous recombination of functionally significant regions of related or different molecules), synthetic proteins or other protein variations including substitutions, deletions, insertion and other variants.

Test agents can be organic or inorganic chemicals, or biomolecules, and all fragments, analogs, homologs, conjugates, and derivatives thereof. Biomolecules include proteins, polypeptides, nucleic acids, lipids, polysaccharides, and all fragments, analogs, homologs, conjugates, and derivatives thereof. Test agents can be of natural or synthetic origin, and can be isolated or purified from their naturally occurring sources, or can be synthesized de novo. Test agents can be defined in terms of structure or composition, or can be undefined. The agents can be an isolated product of unknown structure, a mixture of several known products, or an undefined composition comprising one or more compounds Examples of undefined compositions include cell and tissue extracts, growth medium in which prokaryotic, eukaryotic, and archaebacterial cells have been cultured, fermentation broths, protein expression libraries, and the like.

Test agents such as compounds, drugs, and the like are typically organic molecules, preferably small organic compounds having a molecular weight of more than 100 and less than about 10,000 Daltons, preferably, less than about 2000 to 5000 Daltons. In one embodiment, a small molecule has a molecular weight of less than 1000 Daltons, and typically between 300 and 700 Daltons. Test agents may comprise functional groups necessary for structural interaction with proteins, particularly hydrogen bonding, and typically include at least an amine, carbonyl, hydroxyl or carboxyl group, preferably at least two of the functional chemical groups. The candidate or test agents may comprise cyclical carbon or heterocyclic structures, and/or aromatic or polyaromatic structures substituted with one or more of the above functional groups. Candidate or test agents are also found among biomolecules including peptides, saccharides, fatty acids, steroids, purines, pyrimidines, derivatives, structural analogs or combinations thereof.

In one embodiment, the method (e.g., a high throughput screening assay) involves providing a small organic molecule or peptide library of test agents, the library containing a large number of potential inhibitors. Such “chemical libraries” are then screened in one or more assays, as described herein, to identify those library members (particular chemical species or subclasses) that display a desired characteristic activity. The compounds thus identified can serve as conventional “lead compounds” or can themselves be used as potential or actual products.

In one embodiment, the library of test agents is a combinatorial chemical library. A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Preparation and screening of combinatorial chemical libraries is well known to those of skill in the art. Such combinatorial chemical libraries include, but are not limited to, peptide libraries (see, e.g., U.S. Pat. No. 5,010,175; Furka Int. J. Pept. Prot. Res. 37:487-493 (1991) and Houghton et al., Nature 354:84-88 (1991)). Other chemistries for generating chemical diversity libraries can also be used. Such chemistries include, but are not limited to: peptoids (e.g., PCT Publication No. WO 91/19735), encoded peptides (e.g., PCT Publication No. WO 93/20242), random bio-oligomers (e.g., PCT Publication No. WO 92/00091), benzodiazepines (e.g., U.S. Pat. No. 5,288,514), diversomers such as hydantoins, benzodiazepines and dipeptides (Hobbs et al., Proc. Nat. Acad. Sci. USA 90:6909-6913 (1993)), vinylogous polypeptides (Hagihara et al., J. Amer. Chem Soc. 114:6568 (1992)), nonpeptidal peptidomimetics with glucose scaffolding (Hirschmann et al., J. Amer. Chem Soc. 114:9217-9218 (1992)), analogous organic syntheses of small compound libraries (Chen et al., J. Amer. Chem Soc. 116:2661 (1994)), oligocarbamates (Cho et al., Science 261:1303 (1993)), and/or peptidyl phosphonates (Campbell et al., J. Org. Chem 59:658 (1994)), nucleic acid libraries (see Ausubel, Berger and Sambrook, all supra), peptide nucleic acid libraries (see, e.g., U.S. Pat. No. 5,539,083), antibody libraries (see, e.g., Vaughn et al., Nature Biotechnology, 14:309-314 (1996) and PCTIUS96/10287), carbohydrate libraries (see, e.g., Liang et al., Science, 274:1520-1522 (1996) and U.S. Pat. No. 5,593,853), small organic molecule libraries (see, e.g., isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, U.S. Pat. No. 5,288,514, and the like).

Devices for the preparation of combinatorial libraries are commercially available (see, e.g., 357 MPS, 390 MPS, Advanced Chem Tech, Louisville Ky.; Symphony, Rainin, Woburn, Mass.; 433A Applied Biosystems, Foster City, Calif.; 9050 Plus, Millipore, Bedford, Mass.). In addition, numerous combinatorial libraries are themselves commercially available (see, e.g., ComGenex, Princeton, N.J.; Asinex, Moscow, Russia; Tripos, Inc., St. Louis, Mo.; ChemStar, Ltd, Moscow, Russia; 3D Pharmaceuticals, Exton, Pa.; Martek Biosciences, Columbia, Md.; etc.).

Exemplary embodiments of the various aspects disclosed herein can be described by one of more of the following numbered paragraphs.
1. A pharmaceutical composition comprising:
   a) a compound of Formula I:

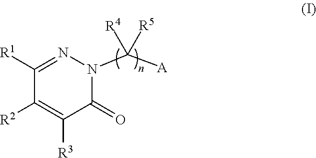

or a pharmaceutically acceptable salt thereof wherein:
R¹, R² and R³ are independently selected from the group consisting of hydrogen, deuterium, halogen, cyano, optionally substituted alkyl, optionally substituted cyclyl, optionally substituted heterocyclyl, optionally substituted aryl, optionally substituted heteroaryl, OR⁶, CO₂R⁶, C(O)NR⁶R⁷, OC(O)R⁶, N(R⁶)C(O)R⁶, NR⁶R⁷, SR⁶, S(O)—R⁶, SO₂R⁶, OS(O)₂R⁶, SO₂NR⁶NR⁷, and NO₂;
R⁴ and R⁵ are independently hydrogen, deuterium, optionally substituted alkyl, or halogen, or R⁴ and R⁵ together with the carbon they are attached to form an optionally substituted cyclic alkyl or optionally substituted heterocyclic;
R⁶ and R⁷ are independently for each occurrence hydrogen, optionally substituted alkyl, optionally substituted cyclyl, optionally substituted heterocyclyl, optionally substituted aryl, or optionally substituted heteroaryl;
A is aryl, heteroaryl, cyclyl, heterocyclyl, or alkyl, each of which can be optionally substituted; and
n is 0, 1, or 2; and
b) a pharmaceutically acceptable carrier.
2. The pharmaceutical composition of paragraph 1, wherein R¹ is hydrogen.
3. The pharmaceutical composition of paragraph 1 or 2, wherein R² is a halogen, NO₂, OS(O)₂R⁶, cyano, hydroxyl, alkoxy, or akylthio.
4. The pharmaceutical composition of any one of paragraphs 1-3, wherein R³ is halogen, heterocyclyl, alkoxy, or alkylamino.
5. The pharmaceutical composition of any one of paragraphs 1-4, wherein R² is a halogen; hydroxyl, alkoxy, or alkylthio; and R³ is a halogen; heterocyclyl; hydroxyl, alkoxy, or alkylthio.
6. The pharmaceutical composition of any one of paragraphs 1-5, wherein R² is Cl, Br, I, F, NO₂, OH, methoxy (—OCH₃), ethoxy (—OEt), mesylate (—OS(O)₂Me), triflate (—OS(O)₂CF₃), besylate (—OS(O)₂Ph), tosylate (—OS(O)₂C6H₄CH₃), methylthio (—SCH₃), or ethylthio (—SCH₂CH₃).
7. The pharmaceutical composition of any one of paragraphs 1-6, wherein R³ is Cl, Br, optionally pyrrolidinyl, methoxy, ethoxy (—OCH₂CH₃) or butylamino (—NH(CH₂)₃CH₃).
8. The pharmaceutical composition of any one of paragraphs 1-7, wherein R² is Cl, and R³ is Cl, methoxy, ethoxy, pyrrolidinyl, or butylamino; R² is hydroxyl, methoxy, or ethylthio, and R³ is Cl; R² and R³ are both Br; or R² and R³ are both methylthio.
9. The pharmaceutical composition of any one of paragraphs 1-8, wherein R¹ is hydrogen, and R² is Cl, and R³ is Cl, methoxy, ethoxy, pyrrolidinyl, or butylamino; R¹ is hydrogen, and R² is hydroxyl, methoxy, or ethylthio, and R³ is Cl; R¹ is hydrogen, and R² and R³ are both Br; or R¹ is hydrogen, and R² and R³ are both methylthio
10. The pharmaceutical composition of any one of paragraphs 1-9, wherein R⁴ and R⁵ are both hydrogen.
11. The pharmaceutical composition of any one of paragraphs 1-10, wherein n is 0 or 1.
12. The pharmaceutical composition of any one of paragraphs 1-11, wherein A is an optionally substituted C₁-C₆alkyl, optionally substituted aryl or optionally substituted heteroaryl.
13. The pharmaceutical composition of paragraph 12, wherein A is an optionally substituted aryl of structure

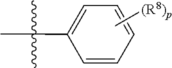

wherein R⁸ is independently for each occurrence deuterium, halogen, cyano, optionally substituted alkyl, optionally substituted cyclyl, optionally substituted heterocyclyl, optionally substituted aryl, optionally substituted heteroaryl, OR⁹, C(O)OR⁹, C(O)NR⁹R¹⁰, OC(O)R⁹, N(R⁹)C(O)R⁹, NR⁹R¹⁰, SR⁹, S(O)R⁹, SO₂R⁹, SO₂NR⁹NR¹⁰, and NO₂, and p is 0, 1, 2, 3, 4, or 5, wherein R⁹ and R¹⁰ are independently for each occurrence hydrogen, optionally substituted alkyl, optionally substituted cyclyl, optionally substituted heterocyclyl, optionally substituted aryl, or optionally substituted heteroaryl.
14. The pharmaceutical composition of paragraph 13, wherein p is 0, 1, 2, or 3.
15. The pharmaceutical composition of paragraph 14, wherein R⁸ is halogen, C₁-C₆alkyl, NO₂, hydroxyl, alkoxy, alkylthio, CF₃, OCF₃, C(O)OR⁹, C(O)NR⁹R¹⁰, or CN.
16. The pharmaceutical composition of any one of paragraphs 12-15, wherein optionally substituted aryl is phenyl; 2-substituted phenyl; 3-substituted phenyl; 2,6-disubstituted phenyl, wherein substituents at the 2-position and 6-position are independently selected; 4-substituted phenyl;’ or 2,3,6-trisubstituted phenyl, wherein substituents at the 2-, 3-, and 6-positions are independently selected.
17. The pharmaceutical composition of paragraph 12, wherein A is an optionally substituted naphthalene.
18. The pharmaceutical composition of paragraph 17, wherein the optionally substituted naphthalene is

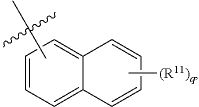

wherein R¹¹ independently for each occurrence deuterium, halogen, cyano, optionally substituted alkyl, optionally substituted cyclyl, optionally substituted heterocyclyl, optionally substituted aryl, optionally substituted heteroaryl, OR¹², C(O)OR¹³, C(O)NR¹²R¹³, OC(O)R¹², N(R¹²)C(O)R¹², NR¹²R¹³, SR¹², S(O)R¹², SO₂R¹², SO₂NR¹²NR¹³, and NO₂, and q is 0, 1, 2, 3, 4, 5, 6, or 7, wherein R¹² and R¹³ are independently for each occurrence are independently for each occurrence hydrogen, optionally substituted alkyl, optionally substituted cyclyl, optionally substituted heterocyclyl, optionally substituted aryl, or optionally substituted heteroaryl.
19. The pharmaceutical composition of paragraph 18, wherein the optionally substituted naphthalene is

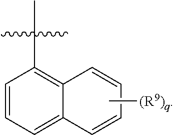

20. The pharmaceutical composition of paragraph 18 or 19, wherein q is 0 or 1.
21. The pharmaceutical composition of paragraph 12, wherein A is an optionally substituted heteroaryl containing 1-2 sulfur, 1-4 nitrogen, or 1-2 oxygen atoms.
22. The pharmaceutical composition of paragraph 21, wherein the optionally substituted heteroaryl is an optionally substituted thiophene, optionally substituted pyridine or optionally substituted pyrimidine.
23. The pharmaceutical composition of any one of paragraphs 1-21, wherein A is selected from the group consisting of methyl, phenyl; 2-bromophenyl; 2-fluorophenyl; 2-chlorophenyl; 2-methylphenyl; 3-methylphenyl; 2-nitrophenyl; 2-cyanophenyl; 2-chloro-6-fluorophenyl; 4-nitrophenyl; 4-chlorophenyl; 4-bromophenyl; 3-methoxyphenyl; 3-cyanophenyl; 2,3,6-trichlorophenyl; 4-aminoformylphenyl; 4-methoxycarbonylphenyl; thiophen-2-yl; 3-chlorothiophen-2-yl; pyridin-2-yl; 3-chloropyridin-2-yl; pyridine-4-yl; 3-chloropyridin-4-yl; naphthalen-1-yl; or 4,6-dimethylpyrimidin-2-yl.
24. The pharmaceutical composition of any one of paragraphs 1-21, wherein the compound of Formula I is a compound from Table 1.
25. The pharmaceutical composition of any one of paragraphs 1-24 further comprising an antibiotic
26. An antibacterial composition comprising a compound of Formula I:

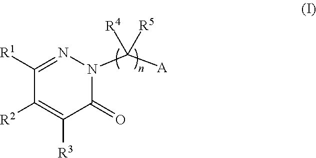

or a pharmaceutically acceptable salt thereof wherein:
R¹, R² and R³ are independently selected from the group consisting of hydrogen, deuterium, halogen, cyano, optionally substituted alkyl, optionally substituted cyclyl, optionally substituted heterocyclyl, optionally substituted aryl, optionally substituted heteroaryl, OR⁶, CO₂R⁶, C(O)NR⁶R⁷, OC(O)R⁶, N(R⁶)C(O)R⁶, NR⁶R⁷, SR⁶, S(O)—R⁶, SO₂R⁶, OS(O)₂R⁶, SO₂NR⁶NR⁷, and NO₂;
R⁴ and R⁵ are independently hydrogen, deuterium, optionally substituted alkyl, or halogen, or R⁴ and R⁵ together with the carbon they are attached to form an optionally substituted cyclic alkyl or optionally substituted heterocyclic;
R⁶ and R⁷ are independently for each occurrence hydrogen, optionally substituted alkyl, optionally substituted cyclyl, optionally substituted heterocyclyl, optionally substituted aryl, or optionally substituted heteroaryl;
A is aryl, heteroaryl, cyclyl, heterocyclyl, or alkyl, each of which can be optionally substituted; and
n is 0, 1, or 2; and
b) a pharmaceutically acceptable carrier.
27. The antibacterial composition of paragraph 26, wherein R¹ is hydrogen.
28. The antibacterial composition of paragraph 26 or 27, wherein R² is a halogen, NO₂, OS(O)₂R⁶, cyano, hydroxyl, alkoxy, or akylthio.
29. The antibacterial composition of any one of paragraphs 26-28, wherein R³ is halogen, heterocyclyl, alkoxy, or alkylamino.
30. The antibacterial composition of any one of paragraphs 26-29, wherein R² is a halogen; hydroxyl, alkoxy, or alkylthio; and R³ is a halogen; heterocyclyl; hydroxyl, alkoxy, or alkylthio.
31. The antibacterial composition of any one of paragraphs 26-30, wherein R² is Cl, Br, I, F, NO₂, OH, methoxy (—OCH₃), ethoxy (—OEt), mesylate (—OS(O)₂Me), triflate (—OS(O)₂CF₃), besylate (—OS(O)₂Ph), tosylate (—OS(O)₂C6H₄CH₃, methylthio (—SCH₃), or ethylthio (—SCH₂CH₃).
32. The antibacterial composition of any one of paragraphs 26-31, wherein R³ is Cl, Br, optionally pyrrolidinyl, methoxy, ethoxy (—OCH₂CH₃) or butylamino (—NH(CH₂)₃CH₃).
33. The antibacterial composition of any one of paragraphs 26-32, wherein R² is Cl, and R³ is Cl, methoxy, ethoxy, pyrrolidinyl, or butylamino; R² is hydroxyl, methoxy, or ethylthio, and R³ is Cl; R² and R³ are both Br; or R² and R³ are both methylthio.
34. The antibacterial composition of any one of paragraphs 26-33, wherein R¹ is hydrogen, and R² is Cl, and R³ is Cl, methoxy, ethoxy, pyrrolidinyl, or butylamino; R¹ is hydrogen, and R² is hydroxyl, methoxy, or ethylthio, and R³ is Cl; R¹ is hydrogen, and R² and R³ are both Br; or R¹ is hydrogen, and R² and R³ are both methylthio
35. The antibacterial composition of any one of paragraphs 26-34, wherein R⁴ and R⁵ are both hydrogen.
36. The antibacterial composition of any one of paragraphs 26-35, wherein n is 0 or 1.
37. The antibacterial composition of any one of paragraphs 26-36, wherein A is an optionally substituted C₁-C₆alkyl, optionally substituted aryl or optionally substituted heteroaryl.
38. The antibacterial composition of paragraph 37, wherein A is an optionally substituted aryl of structure

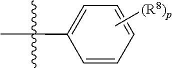

wherein R⁸ is independently for each occurrence deuterium, halogen, cyano, optionally substituted alkyl, optionally substituted cyclyl, optionally substituted heterocyclyl, optionally substituted aryl, optionally substituted heteroaryl, OR⁹, C(O)OR⁹, C(O)NR⁹R¹⁰, OC(O)R⁹, N(R⁹)C(O)R⁹, NR⁹R¹⁰, SR⁹, S(O)R⁹, SO₂R⁹, OS(O)₂R⁶, SO₂NR⁹NR¹⁰, and NO₂; and p is 0, 1, 2, 3, 4, or 5, wherein R⁹ and R¹⁰ are independently for each occurrence hydrogen, optionally substituted alkyl, optionally substituted cyclyl, optionally substituted heterocyclyl, optionally substituted aryl, or optionally substituted heteroaryl.
39. The antibacterial composition of paragraph 38, wherein p is 0, 1, 2, or 3.
40. The antibacterial composition of paragraph 39, wherein R⁸ is halogen, C₁-C₆alkyl, NO₂, hydroxyl, alkoxy, alkylthio, CF₃, OCF₃, C(O)OR⁹, C(O)NR⁹R¹⁰, or CN.
41. The antibacterial composition of any one of paragraphs 37-40, wherein optionally substituted aryl is phenyl; 2-substituted phenyl; 3-substituted phenyl; 2,6-disubstituted phenyl, wherein substituents at the 2-position and 6-position are independently selected; 4-substituted phenyl;’ or 2,3,6-trisubstituted phenyl, wherein substituents at the 2-, 3-, and 6-positions are independently selected.
42. The antibacterial composition of paragraph 37, wherein A is an optionally substituted naphthalene.
43. The antibacterial composition of paragraph 42, wherein the optionally substituted naphthalene is

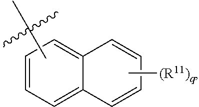

wherein R¹¹ independently for each occurrence deuterium, halogen, cyano, optionally substituted alkyl, optionally substituted cyclyl, optionally substituted heterocyclyl, optionally substituted aryl, optionally substituted heteroaryl, OR¹², C(O)OR¹³, C(O)NR¹²R¹³, OC(O)R¹², N(R¹²)C(O)R¹², NR¹²R¹³, SR¹², S(O)R¹², SO₂R¹², SO₂NR¹²NR¹³, and NO₂; and q is 0, 1, 2, 3, 4, 5, 6, or 7, wherein R¹² and R¹³ are independently for each occurrence are independently for each occurrence hydrogen, optionally substituted alkyl, optionally substituted cyclyl, optionally substituted heterocyclyl, optionally substituted aryl, or optionally substituted heteroaryl.
44. The antibacterial composition of paragraph 43, wherein the optionally substituted naphthalene is

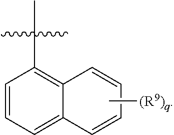

45. The antibacterial composition of paragraph 43 or 44, wherein q is 0 or 1.
46. The antibacterial composition of paragraph 37, wherein A is an optionally substituted heteroaryl containing 1-2 sulfur, 1-4 nitrogen, or 1-2 oxygen atoms.
47. The antibacterial composition of paragraph 46, wherein the optionally substituted heteroaryl is an optionally substituted thiophene, optionally substituted pyridine or optionally substituted pyrimidine.
48. The antibacterial composition of any one of paragraphs 26-47, wherein A is selected from the group consisting of methyl, phenyl; 2-bromophenyl; 2-fluorophenyl; 2-chlorophenyl; 2-methylphenyl; 3-methylphenyl; 2-nitrophenyl; 2-cyanophenyl; 2-chloro-6-fluorophenyl; 4-nitrophenyl; 4-chlorophenyl; 4-bromophenyl; 3-methoxyphenyl; 3-cyanophenyl; 2,3,6-trichlorophenyl; 4-aminoformylphenyl; 4-methoxycarbonylphenyl; thiophen-2-yl; 3-chlorothiophen-2-yl; pyridin-2-yl; 3-chloropyridin-2-yl; pyridine-4-yl; 3-chloropyridin-4-yl; naphthalen-1-yl; or 4,6-dimethylpyrimidin-2-yl.
49. The antibacterial composition of any one of paragraphs 26-48, wherein the compound of Formula I is a compound from Table 1.
50. The antibacterial composition of any one of paragraphs 26-49, further comprising an agent selected from the group consisting of an antibiotic, an antiseptic, and an antifouling agent.
51. A matrix impregnated with a composition of any one of paragraphs 1-50.
52. The matrix of paragraph 51 that is a gel coating specifically formulated for slow release of the antibacterial composition into a surrounding aqueous environment.
53. A method comprising administering a therapeutically effective amount of a pharmaceutical composition of any one of paragraphs 1-25 and 68 to a subject with a bacterial infection.
54. A method of inhibiting growth of a bacteria in a subject comprising administering a therapeutically effective amount of a pharmaceutical composition of any one of paragraphs 1-25 and 68 to the subject.
55. A method of inhibiting growth of a bacteria comprising contacting the bacteria with an effective amount of the antibacterial composition of any one of paragraphs 26-50 and 69.
56. A method of sensitizing a bacteria to growth inhibition comprising contacting the bacteria with an effective amount of the composition of any one of paragraphs 1-50, 68 and 69.
57. A method of inhibiting the development of resistance to an antibiotic by a bacteria comprising, contacting the bacteria with an effective amount of a composition of any one of paragraphs 1-50, 68 and 69 and with an effective amount of the antibiotic.
58. The method of paragraphs 53-57, wherein the bacteria is contacted with Formula I of the composition at a concentration of from about 0.25 μM to about 500 μM.
59. The method of paragraph 53-58, wherein the bacterial is a gram (−) bacteria.
60. The method of paragraph 53-59, wherein the bacteria is a pathogen.
61. The method of any one of paragraphs 53-60, wherein the bacteria is selected from the group consisting of Salmonella typhimurium, Klebsiella pneumoniae, Vibrio cholera, Haemophilus influenza, Francisella tularensis, Klebsiella oxytoca, Enterobacter cloacae, Enterobacter aerogenes, Citrobacter freundii, Pseudomonas aeruginosa, Acinetobacter baumannii, Helicobacter pylori, and combinations thereof
62. A method for identifying an agent that specifically inhibits DsbB, comprising the steps,
   a) testing one or more test agents in a β-gal disulfide bond formation assay using β-gal fused to a bacterial membrane protein, wherein DsbB functions as the oxidant of DsbA in the assay; and
   b) identifying test agents that significantly inhibit disulfide bond formation in the assay; and
   c) further testing the identified test agent(s) in a β-gal disulfide bond formation assay using β-gal fused to a bacterial membrane protein, wherein bVKOR functions as the oxidant of DsbA in the assay;
   wherein the ability of the test agent(s) to significantly inhibit disulfide bond formation in the assay of step a) and the inability of the test agent(s) to inhibit disulfide bond formation in the assay of step c) indicates that the test agent(s) specifically inhibits DsbB.
63. A method for identifying an agent that specifically inhibits bVKOR, comprising the steps,
   a) testing one or more test agents in a β-gal disulfide bond formation assay using β-gal fused to a bacterial membrane protein, wherein bVKOR functions as the oxidant of DsbA in the assay; and
   b) identifying test agents that significantly inhibit disulfide bond formation in the assay; and
   c) further testing the identified test agent in a β-gal disulfide bond formation assay using β-gal fused to a bacterial membrane protein, wherein DsbB functions as the oxidant of DsbA in the assay;
   wherein the ability of the test agent to significantly inhibit disulfide bond formation in the assay of step a) and the inability of the test agent to inhibit disulfide bond formation in the assay of step c) indicates that the test agent specifically inhibits bVKOR.
64. The method of paragraph 62 or 64, wherein the β-gal disulfide bond formation assay is performed as a color assay with bacteria grown on agar that comprise 5-bromo-4-chloro-3-indolyl-β-D-galactopyranoside (BCIG), and color readout is performed by a non-human machine.
65. The method of any one of paragraphs 62-64, wherein the bVKOR is from M. tuberculosis.
66. The method of any one of paragraphs 62-65, wherein the β-gal disulfide bond formation assay is performed in E. coli.
67. The method of any one of paragraphs 62-66, wherein the bacterial membrane protein is MalF.
68. The pharmaceutical composition of any one of paragraphs 1-25 wherein the compound inhibits DsbB of one or more bacteria, and has an IC50 determined with an in vitro E. coli assay with strain DHB7935 of ≦50 μM, ≦25 μM, ≦12 μM, ≦9 μM, ≦8 μM, ≦6 μM, 53 μM, ≦2 μM, ≦1 μM, ≦0.5 μM, ≦0.4 μM, ≦0.3 μM, ≦0.2 μM, ≦0.1 μM, ≦0.09 μM, ≦0.08 μM, ≦0.07 μM, ≦0.06 μM, ≦0.05 μM, ≦0.04 M, ≦0.03 μM, ≦0.02 μM, or ≦0.01 μM.
69. The antibacterial composition of any one of paragraphs 26-50 wherein the compound inhibits DsbB of one or more bacteria, and has an IC 50 determined with an in vitro E. coli assay with strain DHB7935 of ≦50 μM, ≦25 μM, ≦12 μM, ≦9 μM, ≦8 μM, ≦6 μM, ≦≦3 μM, ≦2 μM, ≦1 μM, ≦0.5 μM, ≦0.4 μM, ≦0.3 μM, ≦≦0.2 μM, 50.1 μM, ≦0.09 μM, ≦0.08 μM, ≦0.07 μM, ≦0.06 μM, 0.05 μM, ≦0.04 μM, ≦0.03 μM, ≦0.02 μM, or ≦0.01 μM.

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

It should be understood that this invention is not limited to the particular methodology, protocols, and reagents, etc., described herein and as such may vary. The terminology used herein is for the purpose of describing particular embodiments only, and is not intended to limit the scope of the present invention, which is defined solely by the claims.

Other than in the operating examples, or where otherwise indicated, all numbers expressing quantities of ingredients or reaction conditions used herein should be understood as modified in all instances by the term “about.” The term “about” when used to described the present invention, in connection with percentages means±1%.

In one respect, the present invention relates to the herein described compositions, methods, and respective component(s) thereof as essential to the invention, yet open to the inclusion of unspecified elements, essential or not (“comprising). In one embodiment, other elements to be included in the description of the composition, method or respective component thereof are limited to those that do not materially affect the basic and novel characteristic(s) of the invention (“consisting essentially of”). This applies equally to steps within a described method as well as compositions and components therein. In other embodiments, the inventions, compositions, methods, and respective components thereof described herein are intended to be exclusive of any element not deemed an essential element to the component, composition or method (“consisting of”).

All patents, patent applications, and publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The invention is further illustrated by the following examples, which should not be construed as further limiting.

### Example 1

The following studies arise, at least in part, on the finding that inhibition of disulfide bond formation can be assessed in growing E. coli cells, with either DsbB or VKOR, since inhibition of this process does not prevent cell growth. Added to the advantages of seeking inhibitors of DsbB or VKOR in E. coli is a convenient and very sensitive assay for disulfide bond formation. We have described a version of the enzyme β-galactosidase that is exported to the E. coli periplasm where it is inactivated by disulfide bond formation. This disulfide-sensitive β-galactosidase is the product of a hybrid gene encoding a β-galactosidase fused to a periplasmic domain of the membrane protein MalF as disclosed in U.S. Patent Publication 2011/0243958. In E. coli cells with an intact DsbB/DsbA (or VKOR/DsbA) disulfide bond formation pathway, the level of β-galactosidase activity is two to three orders of magnitude lower than when one or both of the components are absent. This disulfide bond sensitivity is likely due to the formation of disulfide bonds amongst at least some of the 8 pairs of cysteines of β-galactosidase which are normally reduced when the enzyme is in the cytoplasm. Importantly, for studies to be described here, our previous genetic studies have shown that only null mutations in dsbA or dsbB restore high levels of β-galactosidase activity to this hybrid protein. Weaker restoration of β-galactosidase activity results from certain non-null mutations of the dsbA or dsbB genes. In addition, mutations that very weakly restore β-galactosidase activity occur in genes encoding proteins required for cytoplasmic membrane protein assembly. These latter mutations restore only around 1% of the β-galactosidase activity, because presumably null or even strong mutations in these genes would be lethal (Tian, H. P., Boyd, D., and Beckwith, J. Proc. Natl. Acad. Sci. 97:4730-4735 (2000); Kadokura, H., Tian, H., Zander, T., Bardwell, J. C. A. and Beckwith, J. Science 303:534-537 (2004)).

With these tools in hand, we have proceeded to carrying out a high throughput screening procedure, seeking compounds that are potentially useful in the development of antibiotics. The rationale is as follows: 1) Disulfide-bonded proteins are important for bacterial virulence (Heras B, et al. Nat Rev Microbiol. 7:215-25 (2009)); 2) Assaying inhibition of the activity of either Mycobacterium tuberculosis VKOR (Mtb VKOR) or DsbB in vivo can be achieved by assessing their activity in oxidizing DsbA in E. coli; 3) Detecting high levels of activity of the MalF-β-Gal fusion requires strong inhibition by inhibitors of either VKOR or DsbB; 4) From our genetic studies, strong inhibitors of other pathways that restore β-galactosidase activity are not likely be to detected; 5) Screening compounds in parallel for inhibition of VKOR and DsbB provides reciprocal controls that allow us to tentatively eliminate inhibitors that are influencing β-galactosidase activity by interfering with membrane protein assembly or by acting directly on DsbA. Such inhibitors would show up as hits in the screen for both the VKOR and DsbB strains while hits that affect specifically VKOR or specifically DsbB would only show up in the screen for one and not the other of the two proteins.

We here report a screen of ˜51,000 compounds for inhibitors of VKOR or DsbB. A follow-up analysis of candidate inhibitors of either DsbB or VKOR have yielded 6 bona fide effective inhibitors of DsbB and none clearly specifically inhibiting VKOR. Based on the initial group of DsbB inhibitors, we have used structure-activity relationships (SAR) to test a variety of similar compounds and have identified and verified effective inhibitors of DsbB. These inhibitors also inhibit the DsbBs of certain other gram-negative pathogens.

### Results

### A High Throughput Combination Target- and Cell-Based Screen Using Agar-Filled Plates

We initiated high throughput screening for compounds that inhibited disulfide bond formation in two E. coli strains in parallel. One strain (MER672) expresses wild-type levels of DsbB and the other strain (DHB7657) is deleted for the dsbB gene but is complemented by a copy of the M. tuberculosis vkor gene expressed from an IPTG-inducible promoter. Both strains carry the malF-lacZ fusion on the chromosome. In these strains, strong inhibition of disulfide bond formation should lead to a substantial increase in β-galactosidase activity (Tian, H. P., Boyd, D., and Beckwith, J. Proc. Natl. Acad. Sci. 97:4730-4735 (2000); Kadokura et al. Science 303:534-537 (2004); Bardwell et al. Cell. 67:581-589 (1991). Since the enzyme directly responsible for disulfide bond formation in both strains is DsbA, inhibitors of DsbA or of other processes enhancing the activity of β-galactosidase would raise the levels of its activity in both the VKOR-based and DsbB-based strains. In contrast, compounds that specifically inhibited DsbB would show up as increasing β-galactosidase activity in the DsbB-dependent strain but not in the VKOR-dependent strain or vice versa. Assaying the effects of compounds on the two strains in parallel allowed us to pick candidate inhibitors that were specific to either DsbB or VKOR Thus, we have eliminated from further consideration any compounds that gave positive signals with both the VKOR- and DsbB-based strains, even though these might include compounds that did inhibit both enzymes directly.

Initially, we planned to seek compounds that inhibited DsbB or VKOR in 384-well plates containing growing cells in liquid media and, by after a defined time, measuring β-galactosidase activity in each well by assaying with the chromogenic substrate o-nitrophenyl-β-D-galactoside. Because of issues related to the initiation and termination of the enzyme assay we felt it was simpler to use 384-well plates in which each well, in effect, was a mini-agar plate containing growth media rather than a liquid media assay. By including in these agar-filled wells the chromogenic indicator X-Gal (5-bromo-4-chloro-3-indolyl-β-D-galactopyranoside [BCIG]), high levels of β-galactosidase activity appearing in the presence of an inhibitory compound was deduced from the appearance of the blue color resulting from X-Gal hydrolysis. Using such agar-filled wells required special procedures to prevent the solidification of the agar in the tubes supplying it to the plates. Unexpectedly, the analysis presented herein showed that the agar assay is much mere sensitive for detecting inhibitors than the liquid assay and that the DsbB inhibitors we have pursued would not have been detected in the liquid assays.

However, we note that while the wild-type DsbB strain exhibited a white color in the wells with no visible trace of blue, the VKOR-complemented strain did show an observable but very pale blue color, indicating that it did not restore levels of disulfide bond formation comparable to that of the wild-type DsbB strain (Dutton et al. Proc. Natl. Acad. Sci. 107:297-301 (2010). This reduced efficiency in the VKOR-based strain is due to lower levels of expression of VKOR.

The high throughput screen was carried out with 50,374 compounds from the collection of Harvard University's Institute for Chemistry and Chemical Biology (ICCB) and 1113 compounds from the National Institute of Allergy and Infectious Diseases (NIAID) collection of inhibitors of M. tuberculosis H37Rv growth (Ananthan et al. Tuberculosis 89:334-353 (2009); Maddry et al, Tuberculosis 89:354-363 (2009). Each strain assay for each compound was performed in duplicate. While for DsbB, hits were readily identified by the clear blue/white difference, for VKOR, distinguishing weak hits from the light blue background color was not so clear because of the incomplete VKOR complementation. We set a very low threshold for assigning hits (i.e. anything bluer than the background in at least one of the duplicate wells). In the initial screen of 51,487 compounds, we identified 11 and 150 potential inhibitors of DsbB and VKOR, respectively. The much higher number of inhibitors observed for the VKOR strain is likely due to its lessened efficiency in oxidizing DsbA, which makes the strain more sensitive to weak inhibitory effects on VKOR, DsbA or proteins involved in membrane protein assembly.

To verify as potential inhibitors the compounds identified in our initial screen, we retested them in the same 384-well plate format. After this step, the numbers of potential inhibitors of DsbB and VKOR was reduced to 8 (corresponding to a hit rate of 0.016%) and 62 (0.12%), respectively. Further initial analysis of the compounds included 1) discussions with medicinal chemists regarding potential utility of particular compounds based on their likely broad reactivity; 2) finding that some reordered samples of compounds failed to replicate their inhibitory effects; and 3) finding that, upon more careful retesting, a few compounds showed inhibition of both DsbB and VKOR. These factors eliminated more compounds from further consideration and left us with three potential inhibitors of VKOR (1, 4, and 8) and six potential inhibitors of DsbB (12-17).

Testing for Candidate Inhibitors of M. tuberculosis VKOR

We examined several properties of the potential VKOR inhibitors (1, 4 and 8) to determine whether any of them might be pursued as possible lead compounds for antibiotic development against tuberculosis. Since VKOR is essential for growth of M. tuberculosis, we tested these three compounds, all of the potential DsbB inhibitors (12-17), and several of the inhibitors of M. tuberculosis H37Rv growth from the NIAID collection for their growth inhibitory effects in three different media. Not surprisingly, all of the potential VKOR inhibitors that had been obtained from the NIAID collection strongly inhibited M. tuberculosis growth in at least one of the media used and usually all three. Of the 6 DsbB inhibitors from the DsbB screen that were of interest (12-17), some showed no degree of inhibition of M. tuberculosis growth while others showed some M. tuberculosis growth inhibition, although considerably less than that seen with the NIAID collection of M. tuberculosis growth inhibitors. Nevertheless, the inhibitors from the DsbB screen that did show some indications of growth inhibition in this test were the stronger of the DsbB inhibitors (Table 5). At any rate, of the three inhibitors of the VKOR-based strain remaining, compounds 4 and 8 showed no effects on growth of M. tuberculosis growth and compound 1 showed a relatively strong effect in only one of the three media. Compounds 4 and 8 were eliminated from continued study.

In considering the utility of potential inhibitors of Mtb VKOR, we recognized that an antibiotic against M. tuberculosis should not also have anti-coagulant activity (such as warfarin does). To test compounds for anti-coagulant activity, we assessed the activity of some of these compounds in inhibiting mouse VKORc1, the homologue of MtbVKOR that is the enzyme involved in blood coagulation. At the same time, to obtain some evidence whether any inhibition seen of VKORc1 was specific and not due to the ability of these compounds to react with proteins with redox-active cysteines, we determined whether the compounds interfered with the activity of such proteins more generally. Therefore, in addition to VKORc1 activity, we assayed the effect of these compounds on the activity of endoplasmic reticular proteins PDI, Erp5, Erp57 and Erp72. Results of these studies showed that compound 1 inhibited the activity of VKOR and each of the ER proteins. The broad inhibitory activity toward thiol redox proteins eliminated compound 1 as a candidate specific to VKOR.

From these various tests, we eliminated all of the potential inhibitors of VKOR from further study as inhibitors of M. tuberculosis growth and virulence via their interactions with Mtb VKOR However, we did discover in the early stages of screening with a collection of bioreactive compounds provided by the ICCB, that one of the hits with VKOR was the compound brominedione. Brominedione is a known anti-coagulant and inhibitor of human VKORc1. It showed up in the screen as a strong inhibitor of the MtbVKOR-based strain but not of the DsbB-based strain. This one positive result with MtbVKOR from our initial screening indicates that the screen could be used to identify new classes of anti-coagulants as well as the M. tuberculosis inhibitors we were seeking.

[symbol]

TABLE 5
Effect on Mycobacterium tuberculosis growth with VKOR and
DsbB inhibitors.

| | | [symbol] | | | |
|---|---|---|---|---|---|
| [symbol] | [symbol] | [symbol] | [symbol] | [symbol] | [symbol] |

[symbol]

inhibitors

| | | | | | |
|---|---|---|---|---|---|
| 1 | 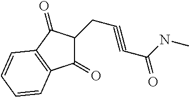 [SMILES: CNC(=O)C#CCC1C(=O)c2ccccc2C1=O] | 128 | 510 | 1000 | 510 |
| 4 | 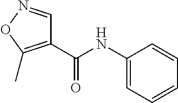 | 1100 | >1100 | >1100 | >1100 |
| 0 | 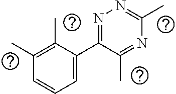 | >900 | >900 | >900 | >900 |
| 23 | 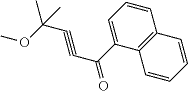 [SMILES: COC(C)(C)C#CC(=O)c1cccc2ccccc12] | 52 | 120 | 120 | 120 |

[symbol]

inhibitors

| | | | | | |
|---|---|---|---|---|---|
| 12 | 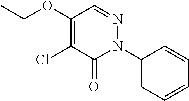 [SMILES: CCOc1cnn(C2C=CC=CC2)c(=O)c1Cl] | 470 | >540 | 940 | 940 |
| 13 | 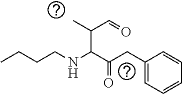 [SMILES: CCCCNC(C(=O)Cc1ccccc1)C(C)C=O] | 900 | >900 | 900 | 900 |
| 14 | 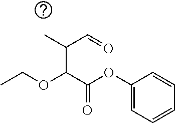 [SMILES: CCOC(C(=O)Oc1ccccc1)C(C)C=O] | 500 | 1000 | 1000 | 1000 |
| 15 | 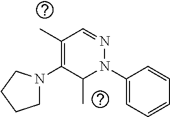 [SMILES: CC1=C(N2CCCC2)C(C)N(c2ccccc2)N=C1] | 450 | 910 | 910 | 910 |
| 16 | 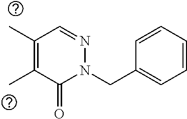 [SMILES: Cc1cnn(Cc2ccccc2)c(=O)c1C] | 250 | 490 | 490 | 250 |
| 17 | 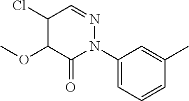 | >1000 | >1000 | >1000 | >1000 |
| 18 | 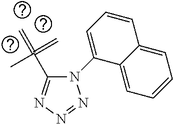 | 52 | 110 | 110 | 110 |

[symbol]

indicates data missing or illegible when filed

### Characterization of Potential DsbB Inhibitors

Of the eight inhibitors in the DsbB screen, one of them (compound 18) was eliminated because it also had some inhibitory activity in the VKOR screen and caused growth defects with M. tuberculosis. Compound 23, detected in the VKOR screen was a weak inhibitor of DsbB and an inhibitor of M. tuberculosis growth. We eliminated these two from further study at this point since the lack of specificity and the growth inhibition indicated that the effects of these compounds might be due to a broader reactivity affecting other cellular components. The remaining 6 potential DsbB inhibitors (12-17) did not inhibit VKOR-promoted disulfide bond formation in E. coli, making it likely that they directly inhibited DsbB. These compounds shared a common structural feature, a pyridazinone ring with different substituents at position 2, 4 and 5.

In order to determine the concentrations at which these compounds inhibit DsbB, we used two assays. In our initial assay, although we realized that we could not calculate real concentrations of inhibitor in the agar media plate assays, we, nevertheless, did first calculate MICs for the inhibitors with the agar media plates by this approach, in part, to determine which were the strongest inhibitors. Second, we measured MICs of the strongest inhibitors in terms of their effects on the oxidation of DsbA by DsbB and on the inhibition of disulfide bond formation in growing liquid cultures. We grew a wild-type E. coli strain in liquid minimal medium and determined the minimal inhibitory concentration at which we could observe 1) increased presence of reduced DsbA due to inhibition of oxidization of DsbA by DsbB and 2) inhibition of disulfide bond formation in RcsF, a substrate of DsbA. The oxidation of DsbA was assessed by a standard alkylation assay for free cysteines in DsbA. With the apparently strongest inhibitor, compound 16, we obtained MICs of 5 μM (DsbA) and 100 μM (RcsF) for the two assays. Interestingly, the MIC obtained for blue color appearance in β-galactosidase agar media assay is 5.7 μM. The remaining five compounds gave significantly higher MICs ranging from 8 to 31 μM in the latter assay. The direct measurement of DsbA's oxidation state gives the best quantitative sense of the effect of inhibitors, while variation in MICs in the other assays is presumably due to the varying efficiency with which DsbA oxidizes different substrate proteins.

In addition to the in vivo assays of MICs, we have purified DsbB from E. coli and assessed the concentration dependence for inhibition of the enzyme's activity in oxidizing DsbA. This yielded for inhibitor #16 an IC50 of 1.85 μM which had appeared to be the most potent inhibitor obtained in the high throughput screen.

### Structure-Activity (SAR) Relationship Analysis of DsbB Inhibitors

The common structural features of the six DsbB inhibitors obtained in the high throughput screening led us to seek out available compounds with similar structures that we could test for ones that had enhanced activity over our most active compound #16. We first asked whether any of the 50,000 compounds already screened that did not show up as inhibitors of DsbB had similar structures (pyridazinone) to the inhibitors detected. We found 46 such compounds, four of which were very weak inhibitors (detected with a strain expressing lower levels than wild-type of DsbB (DHB7935), see Materials and Methods), and which were not pursued further. We also examined the list of the remaining 549,283 compounds of the ICCB collection and found 5 with related structure, 3 of which inhibited DsbB in our assay. From a survey of the array of compounds that were not inhibitors and from the commonalties found among the effective inhibitors, we carried out structure-activity relationships and ordered 24 additional compounds to assess chemical changes in the pyridazinone structure that may give stronger inhibitors of DsbB. Amongst the approaches we used to choose compounds to order were 1) varying the halogen groups linked to the pyridazinone ring moieties from chlorine to bromine or fluorine as well as other small polar and cyclic groups at positions 4 and 5; 2) introducing one to three halogens in the benzyl ring of compound 16 such as chlorine, bromine or fluorine; 3) adding other groups different than halogens in the benzyl ring such as small alkyl, cyano, amide, amine and carboxyl groups; 4) changing the benzyl ring to phenyl at position 2 of the pyridazinone in order to shorten the distance between the two rings and explore as well halogen substituents in the phenyl group. Amongst these variants on the common structure, we found 4 compounds that had DsbB inhibitory activity that showed inhibition as good as or better than the compounds picked up in the high throughput screening. These compounds are shown in Table 6. We obtained inhibitors that we named 16.12 and 16.6 (10-fold and 23-fold more inhibitory than compound 16, respectively). Compound 16.6 has a Ki of 2 nM in the in vitro assay and an MIC of 0.7 μM in inhibiting DsbA oxidation in growing E. coli.

TABLE 6
Strongest inhibitors obtained after SAR-analysis.

| ID | | EC50 RATIO |
|---|---|---|
| Num- | | (EC50 compound |
| ber | STRUCTURE | 16/EC50) |
| 16.6 | 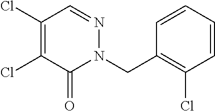 [SMILES: O=c1c(Cl)c(Cl)cnn1Cc1ccccc1Cl] | 23.03 |
| 16.12 | 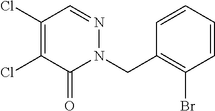 [SMILES: O=c1c(Cl)c(Cl)cnn1Cc1ccccc1Br] | 10.77 |
| 16.20 | 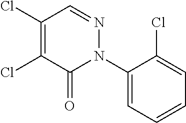 [SMILES: O=c1c(Cl)c(Cl)cnn1-c1ccccc1Cl] | 3.80 |
| 16.2 | 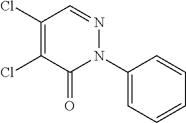 [SMILES: O=c1c(Cl)c(Cl)cnn1-c1ccccc1] | 2.49 |
| 16.23 | 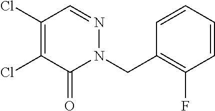 [SMILES: O=c1c(Cl)c(Cl)cnn1Cc1ccccc1F] | 1.65 |
| 16.13 | 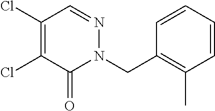 [SMILES: Cc1ccccc1Cn1ncc(Cl)c(Cl)c1=O] | 1.43 |
| 16 | 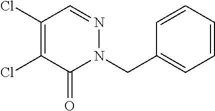 [SMILES: O=c1c(Cl)c(Cl)cnn1Cc1ccccc1] | 1.00 |
| 16.24 | 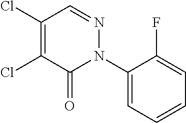 [SMILES: O=c1c(Cl)c(Cl)cnn1-c1ccccc1F] | 0.73 |
| 16.14 | 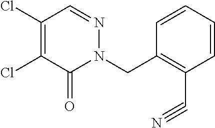 [SMILES: N#Cc1ccccc1Cn1ncc(Cl)c(Cl)c1=O] | 0.39 |
| 16.4 | 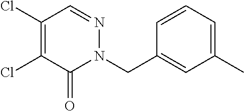 [SMILES: Cc1cccc(Cn2ncc(Cl)c(Cl)c2=O)c1] | 0.37 |

Further Evidence that DsbB Inhibitors do not Inhibit VKOR

Although our screening procedures so far eliminated compounds that inhibited both DsbB and VKOR, to be certain, we tested systematically the entire collection of pyridazinone compounds that inhibited DsbB for inhibition of VKOR. We assayed inhibition by the same blue/white agar assay using the tester E. coli strains expressing either VKOR of M. tuberculosis or DsbB. We did not observe inhibition of VKOR at concentrations of up to 100 μM of any one of these compounds. This result strengthens our conclusion that the inhibition of DsbB by the pyridazinone compounds is specific and that they may not inhibit VKORs from other organisms, including the human enzyme. We have previously shown that the known inhibitors of human VKORc1L1 tested also inhibited MtbVKOR including brominedione which does not inhibit DsbB (see above) and others (Dutton, R. J., Wayman, A., Wei, J.-R., Rubin, E. J., Beckwith, J., and Boyd, D. Inhibition of bacterial disulfide bond formation by the anti-coagulant warfarin. Proc. Natl. Acad. Sci. 107:297-301 (2010); Li, W., Schulman, S., Dutton, R. J., Boyd, D., Beckwith, J., and Rapoport, T. A. Structure of a bacterial homolog of vitamin K epoxide reductase. Nature 463:507-512 (2010)).

Testing the Response of DsbBs from Other Gram-Negative Pathogens to Identified Inhibitors of E. coli DsbB

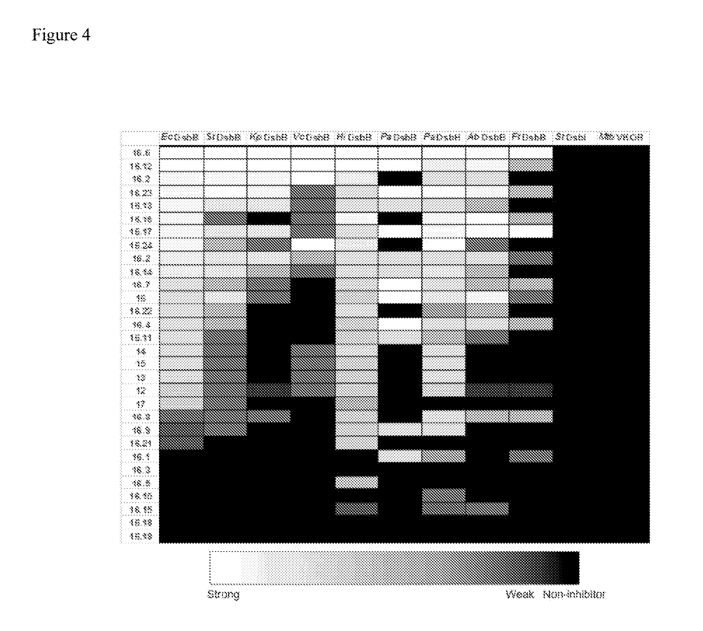

We proceeded to ask whether the inhibitors of E. coli DsbB identified here would also inhibit DsbBs from other gram-negative pathogens. We chose to do this first by determining whether these DsbBs complement an E. coli strain deleted for its dsbB gene. If the DsbBs from other organisms could effectively replace E. coli DsbB, we could then assay the effects of inhibitors on these DsbBs in growing E. coli via the β-galactosidase assay used in the high throughput screening. If inhibition of any one of these DsbBs is observed in this assay, we could proceed to determine the effects of the compound(s) on disulfide bond formation in the gram-negative pathogens themselves. We cloned the dsbB genes from Acinetobacter baumanni, Klebsiella pneumonia, Vibrio cholerae, Francisella tularensis, Haemophilus influenzae as well as the two homologues of dsbB (dsbB and dsbH) from Pseudomonas aeruginosa and two homologues (dsbB and dsbl) from Salmonella typhimurium into a plasmid where they were expressed from a weakened Trc₂₀₄ promoter [David S. Weiss, Joseph C. Chen, Jean-Marc Ghigo, Dana Boyd, and Jon Beckwith. J Localization of FtsI (PBP3) to the Septal Ring Requires Its Membrane Anchor, the Z Ring, FtsA, FtsQ, and FtsL J. Bacteriology, 181(2): 508-520. (1999)]. When cloned into E. coli, all DsbB homologues maintained the disulfide-sensitive β-galactosidase in the disulfide-bonded state as indicated by the absence of blue color in agar growth media containing X-GaL The DsbH of P. aeruginosa, the DsbB from A. baumanni, the DsbI from S. typhimurium and the DsbB from F. tularensis required higher levels of expression to cause the E. coli colonies to show strong inactivation of β-galactosidase as indicated by absence of XGal hydrolysis. Surprisingly, the Klebsiella DsbB was effective at lower induction levels than E. coli DsbB. Western blots with anti-E. coli DsbB showed higher levels of the Klebsiella DsbB than E. coli DsbB when both were expressed from the same promoter at the same inducer level. Each of the E. coli ΔdsbB mutant strains complemented by the other gram-negative DsbBs was tested for their sensitivity to the collection of characterized inhibitors of E. coli DsbB and to some of the non-inhibitory pyridazinone compounds. Several dilutions of solutions of these inhibitors were dropped onto the agar media containing XGal in 384-well plates with the ΔdsbB strain complemented by the different DsbBs. The set of 3 compounds that did not inhibit E. coli DsbB also did not inhibit the DsbBs of the other gram-negative bacteria expressed in E. coli. However, amongst the collection of compounds that did inhibit E. coli DsbB, we found some that also inhibited the other DsbBs. While the most effective inhibitor of them was 16.6, the DsbH of P. aeruginosa, the DsbBs from A. baumanni and F. tularensis were more effectively inhibited by other compounds of this group (FIG. 4).

### Discussion

We have described a methodology for high throughput screening of chemical compounds for inhibition of the disulfide bond-forming pathways of the gram-negative bacterium Escherichia coli and the gram-positive bacterium Mycobacterium tuberculosis. The target components in these screens are the enzyme DsbB of E. coli and its counterpart VKOR in M. tuberculosis. Both of these enzymes are required for the regeneration, in their respective organisms, of active DsbA which is the enzyme that directly introduces disulfide bonds into substrate proteins.

The principles of our approach have novel aspects, which may be useful in other types of screens. First, we are not aware of high throughput screens with bacteria that employ agar in their wells instead of liquid media. While there was a learning curve in maintaining just the right temperature and timing to prevent clogging the robot tubes that delivered the agar to the wells, this issue has been successfully worked out.

Second, the detection of activity of a compound inhibitory of disulfide bond formation is readily observed as the appearance of blue color exhibited by bacteria growing on agar media in a well of a 384-well plate. The remaining wells in which no inhibition takes place exhibit no color (in the case of the DsbB inhibitors). We could simply observe the color by eye to readily detect what were mostly real inhibitors with practically no false positives.

Third, the procedure that we use to drop agar media and bacteria, and then the compound to be tested into the agar wells of 384-well plates likely results in higher concentrations of the compounds where the bacteria are growing than one might calculate since compounds first concentrate where they are dropped. This consequence of the approach results in the assay being more sensitive than we had anticipated it would be. This sensitivity is needed as it takes very strong inhibition of DsbB to generate enough β-galactosidase to give a strong, readily visible blue color. The requirement for high compound concentration may be due to the β-galactosidase remaining inactivated by disulfide bonds until DsbA is nearly completely inhibited. This sensitivity may be a consequence of fact that that β-galactosidase has 16 cysteines and that formation of only one or a few disulfide bonds between pairs of cysteines in the protein may be sufficient to inactivate the enzyme. Thus, even the quite low activities of DsbA activity manifested in the presence of nearly completely inhibitory activity of compounds must be sufficient to promote at least a single disulfide bond in the β-galactosidase. These findings are consistent with our earlier findings that selection of mutants that restore full expression of β-galactosidase require mutations of DsbA or DsbB that completely remove those enzymes' activity.

Fourth, the screening assay in E. coli which depends on the DsbA protein as the substrate of an enzyme (DsbB) that can reoxidize reduced DsbA, allows additional screens in which the native DsbB is replaced by DsbBs from numerous gram-negative bacteria and with the enzyme VKOR from other bacteria such as M. tuberculosis. Further, if the VKOR of vertebrates including that of humans can be expressed in E. coli as a functional replacement for DsbB, the human VKOR could be used in this assay to screen for new classes of blood thinners. In each screen, the specificity of the inhibitors can be checked by including a parallel screen where the compounds are tested against another DsbA oxidant, e.g. E. coli DsbB.

The screen described here with VKOR probably yielded a much higher number of hits that were not direct inhibitors of VKOR compared to the DsbB screen where most of the compounds were direct inhibitors of DsbB. This difference was indicated first by the fact that the DsbB screen yielded only 11 possible positive hits, while that with VKOR yielded 150 candidate compounds We believe that the reason for this disparity is that the DsbA-oxidizing activity in the Mtb VKOR screening strain is low compared to that of the DsbB screening strain. For instance, we observe a distinct, albeit quite pale, blue color in the wells containing the VKOR expressing strains as compared to the white color of growing bacteria in the DsbB strain. However, when the VKOR is expressed from a plasmid, the bacterial colonies are white (Dutton et al. Proc. Natl. Acad. Sci. 107:297-301 (2010)). Based on the results with the DsbB strain, we suspect that further screens for VKOR inhibitors using a strain carrying this plasmid will reduce the number of weak and indirect hits making it easier to process the compounds for those that are strong specific inhibitors of VKOR

The DsbB screen yielded 6 compounds that were inhibitors of DsbB at micromolar concentrations and 20 other inhibitors were detected by SAR some of which inhibited in the nanomolar range. None of these compounds inhibited VKOR even at much higher concentrations. For instance, the minimal inhibitory concentration of DsbB by compound 16.6 is 0.53 μM, whereas no inhibition by 16.6 of VKOR is observed at concentrations as high as 50 μM, giving a difference of equal to or greater than two orders of magnitude. Since the VKOR is expressed at lower levels in these studies, the difference is certainly greater than this. These findings indicate that the DsbB inhibitors obtained are specific in that they affect the activity of DsbB but not VKOR, despite the identical mechanisms of action of the two proteins.

Bioinformatic searches indicate that neither DsbB nor bacterial VKOR are parts of families of commonly found proteins, in contrast to DsbA which is part of the huge family of thioredoxin-like proteins. No homologues of DsbB have been found so far in eukaryotes. The only VKOR family members identified in vertebrates such as humans are ones involved in coagulation, VKORc1L1, and the VKORc1L1-like protein perhaps involved in redox-maintenance. This may mean that inhibitors specific to DsbB (as well as to VKOR) in microbial pathogens may have few other targets they can interact with in humans that could compromise their use as antibiotics.

Our SAR survey of the inhibitory effectiveness of a number of pyridazinone compounds suggests that the potency of an inhibitor may be related to the high electron affinity given by the presence of chlorine groups as well as the presence of a second aromatic ring either phenyl or benzyl group. The inhibitors may act by competing with quinone binding, thus blocking the transfer of electrons from DsbB to the quinone.

E. coli cells lacking DsbB or DsbA are unable to grow under anaerobic conditions, although they do grow aerobically. These findings indicate that the inhibitors described here will not interfere with aerobic growth of many other proteobacteria bacteria. In fact, we have already shown that aerobic growth of P. aeruginosa is not inhibited by the compounds that were tested, despite the fact that dsb mutants reduce virulence. However, a failure to grow anaerobically in combination with defects in expressing active virulence factors may, for some pathogens, add to the effectiveness of these compounds as antibiotics. Recently reports have argued that targeting the virulence of a pathogen without necessarily targeting normal growth may be an attractive option for developing new antibiotics. It is suggested that there would be weaker selective pressure for the development of resistance and potentially more likelihood of clearance by the host immune response (Clatworthy A. E et al. Nature Chemical Biology 3:541-548. (2007)). Although, eventually the organism would develop resistance to the inhibitor, we think that these inhibitors could be used in combination with other antibiotics to maximize the effect and to complicate the development of resistance.

### Materials and Methods

### Strain Construction.

The strains and plasmids used in this study are listed in Table 7. The malF-lacZ102 fusion with Kanamycin resistance (derived from pDHB5700 (Froshauer et al., J Mol Biol. 200: 501-511 (1988); Boyd et al. J. Bacteriol. 182(3): 842-847 (2000)) was integrated into the chromosome of HK295 and HK320 strains by the λInCh method (Boyd et al., J. Bacteriol. 182(3): 842-847 (2000)) to generate HK314 and HK325 strains, respectively. MER672 and DHB7658 were constructed inserting pTrc99a (empty vector) at the recombined λatt site by λInCh into the chromosome of HK314 and HK325 strains, respectively. In order to generate DHB7657 strain instead pTrc99aMtbVKOR (pRD33, Dutton et al., Proc. Natl. Acad. Sci. 107:297-301 (2010)) was moved to HK325 by λInCh. In order to stabilize both insertions at the λ attachment site, the recA⁻ mutation (BW10724, Keio collection) was moved by P1 transduction into the three strains. Strains DHB7935 and DHB7936 were constructed by introducing into the chromosome plasmids pDSW206dsbBhis6-c-myc (pDHB7933) and pDSW206 (empty vector) at the 080 attachment site of HK325 as described previously [Haldimann A. and Wanner B. L. Conditional-replication, integration, excision, and retrieval plasmid-host systems for gene structure-function studies of bacteria. J Bacteriol. 183(21):6384-93. (2001)]. To generate CL315 and CL320, a PCR product that extends from the lacI gene to the ampicillin-resistance cassette (primers C165 and C166) of pCL25 (P. aeruginosa dsbB) and pCL24 (K. pneumoniae dsbB) plasmids were introduced into the ΔdsbB loci of HK320 strain using λ Red proteins expressed from pCL58. Then, each insertion was moved to HK325 strain by P1 transduction. The other strains expressing different dsbB genes were obtained by transformation of the respective plasmid into HK325. All of the dsbB-complemented strains were verified by their motility in 0.3% agar minimal media and adjusted in Xgal minimal media plates to levels of IPTG that resulted in white colonies, i.e. complementing the dsbB mutant phenotype. Thus, the IPTG concentrations used were: 50 μM for the E. coli strain expressing PadsbH and AbdsbB, 75 μM for the strain expressing StdsbI and 2 mM for the strain expressing FtdsbB. For strains expressing KpdsbB, PadsbB, StdsbB, VcdsbB and HidsbB genes the basal levels of expression were enough to complement so, no IPTG was required to add.

All strains were grown in NZ or in M63 broth and agar media at 30° C. when indicated. The antibiotic concentrations used were: ampicillin 25 μg/ml or 100 μg/ml, kanamycin 40 μg/ml and chloramphenicol 10 μg/ml.

TABLE 7
Strains used in this work

| | | |
|---|---|---|
| Strain | Genotype | Source |

Escherichia coli strains

| | | |
|---|---|---|
| HK295 | MC1000 Δara714 leu⁺ | Kadokura et al., EMBO J. |
| | | 21(10): 2354-2363. (2002) |
| HK320 | HK295 ΔdsbB | Kadokura et al., EMBO J. |
| | | 21(10): 2354-2363. (2002) |
| HK314 | HK295 λatt::malF-lacZ102 (Km^{r}) | H. Kadokura |
| HK325 | HK295 ΔdsbB λatt::malF-lacZ102 (Km^{r}) | H. Kadokura |
| MER672 | HK295 λatt::malF-lacZ102 (Km^{r}) | This study |
| | pTrc99a (Amp^{r}) recA::Cm | |
| DHB7657 | HK295 ΔdsbB λ::malF-lacZ102 (Km^{r}) | This study |
| | pTrc99aMtbVKOR (Amp^{r}) recA::Cm^{r} | |
| DHB7658 | HK295 ΔdsbB λ::malF-lacZ102 (Km^{r}) | This study |
| | pTrc99a (Amp^{r}) recA::Cm^{r} | |
| DHB7935 | HK295 ΔdsbB λatt::malF-lacZ102 (Km^{r}) | This study |
| | φ80::pDSW206dsbBhis6-c-myc (Amp^{r}) | |
| DHB7936 | HK295 ΔdsbB λatt::malF-lacZ102 (Km^{r}) | This study |
| | φ80::pDSW206 (Amp^{r}) | |
| CL315 | HK295 λatt::malF-lacZ102 (Km^{r}) | This study |
| | ΔdsbB::pDSW204PadsbB (Amp^{r}) | |
| CL320 | HK295 λatt::malF-lacZ102 (Km^{r}) | This study |
| | ΔdsbB::pDSW204KpdsbB (Amp^{r}) | |
| CL377 | HK295 ΔdsbB λatt::malF-lacZ102 (Km^{r}) | This study |
| | pDSW204PadsbH (Amp^{r}) | |
| CL378 | HK295 ΔdsbB λatt::malF-lacZ102 (Km^{r}) | This study |
| | pDSW204AbdsbB (Amp^{r}) | |
| CL369 | HK295 ΔdsbB λatt::malF-lacZ102 (Km^{r}) | This study |
| | pDSW204StdsbB (Amp^{r}) | |
| CL368 | HK295 ΔdsbB λatt::malF-lacZ102 (Km^{r}) | This study |
| | pDSW204StdsbI (Amp^{r}) | |
| CL373 | HK295 ΔdsbB λatt::malF-lacZ102 (Km^{r}) | This study |
| | pDSW204VcdsbB (Amp^{r}) | |
| CL370 | HK295 ΔdsbB λatt::malF-lacZ102 (Km^{r}) | This study |
| | pDSW204FtdsbB (Amp^{r}) | |
| CL371 | HK295 ΔdsbB λatt::malF-lacZ102 (Km^{r}) | This study |
| | pDSW204HidsbB (Amp^{r}) | |
| CL379 | HK295 ΔdsbB λatt::malF-lacZ102 (Km^{r}) | This study |
| | pDSW204 | |

Mycobacterium smegmatis strains

| | | |
|---|---|---|
| RD263 | M. smegmatis Δvkor transformed with | Dutton, et al., Proc. Natl. Acad. |
| | pRD43 (pTetG-E. coli dsbB) | Sci. 107: 297-301 (2010) |
| RD265 | M. smegmatis Δvkor transformed with | Dutton, et al., Proc. Natl. Acad. |
| | pRD42 (pTetG-Mtb vkor) | Sci. 107: 297-301 (2010) |

Plasmids

| | | |
|---|---|---|
| pTrc99a | Expression vector, Amp^{r} | Amann et al. Gene 69: 301-315 |
| | | (1988) |
| pDSW206 | Promoter down mutation in −10 and | Weiss et al., J. Bacteriol. 181: 508- |
| | −35 of pTrc99a | 520 (1999) |
| pKD46 | Encodes lambda Red genes under | Datsenko K. A. and Wanner B. L. |
| | arabinose promoter, Amp^{r} | Proc Natl Acad Sci U.S.A. |
| | | 97(12): 6640-5. (2000) |
| pRD33 | pTrc99a-hisMtbVKOR | Dutton, et al. Proc. Natl. Acad. |
| | | Sci. 107: 297-301 (2010) |
| pDHB7933 | pDSW206dsbB-his6-c-myc | This study |
| pCL58 | pKD46 Amp^{r} replaced by Cm^{r} | This study |
| pCL25 | pDSW204PadsbB (Pseudomonas | This study |
| | aeruginosa UCBPP-PA14 dsbB gene, | |
| | PA14_07000) | |
| pCL26 | pDSW204PadsbH (Pseudomonas | This study |
| | aeruginosa UCBPP-PA14 dsbH gene, | |
| | PA14_69400) | |
| pCL24 | pDSW204KpdsbB (Klebsiella | This study |
| | pneumoniae W63917 dsbB gene) | |
| pCL27 | pDSW204AbdsbB (Acinetobacter | This study |
| | baumannii A118 dsbB gene) | |
| pCL62 | pDSW204StdsbB (Salmonella enterica | This study |
| | subsp. enterica serovar Typhimurium str. | |
| | LT2 dsbB gene, STM1807) | |
| pCL61 | pDSW204StdsbI (Salmonella enterica | This study |
| | subsp. enterica serovar Typhimurium str. | |
| | LT2 dsbI gene, STM3194) | |
| pCL66 | pDSW204VcdsbB (Vibrio cholerae O1 | This study |
| | biovar El Tor str. N16961 dsbB gene, | |
| | VC1902) | |
| pCL63 | pDSW204FtdsbB (Francisella tularensis | This study |
| | subsp. holartica LVS dsbB gene, | |
| | FTL1670) | |
| pCL64 | pDSW204HidsbB (Haemophilus | This study |
| | influenzae RdKW20 dsbB gene, HI0428) | |

TABLE 8
Primers used in this study

| | | | Restriction |
|---|---|---|---|
| Name | Gene | Sequence | sites |
| Cl16 | KpdsbB-1 | Gcgttcatgatgttg | BspHI |
| | | caatatttaaaccag | |
| | | tgctca | |
| | | (SEQ ID NO: 2) | |
| Cl17 | KpdsbB-2 | Cggagctcttaacga | SacI |
| | | ccaaacagatcgcgt | |
| | | t | |
| | | (SEQ ID NO: 3) | |
| Cl3 | PadsbB-1 | Tcgaagctttcaggc | HindIII |
| | | ggtgcggcggcc | |
| | | (SEQ ID NO: 4) | |
| Cl10 | PadsbB-2 | Gctgtcatgagcagc | BspHI |
| | | gctctcctcaa | |
| | | (SEQ ID NO: 5) | |
| CL5 | PadsbH-1 | Cagaagctttcaggc | HindIII |
| | | acgtcggaggaac | |
| | | (SEQ ID NO: 6) | |
| Cl9 | PadsbH-2 | Ctccatggtgcccct | NcoI |
| | | ggccagcccc | |
| | | (SEQ ID NO: 7) | |
| Cl19 | AbdsbB-1 | Ctccatggtgcgatt | NcoI |
| | | aagttaccgtttggt | |
| | | (SEQ ID NO: 8) | |
| Cl20 | AbdsbB-2 | Cggagctcttacttt | SacI |
| | | ttagccgtcttaa | |
| | | (SEQ ID NO: 9) | |
| Cl92 | StdsbB-1 | Gactccatgggccat | NcoI |
| | | tatttcatttcccgc | |
| | | tagtggcg | |
| | | (SEQ ID NO: 10) | |
| Cl93 | StdsbB-2 | Cgtcggatccgatgt | BamHI |
| | | atttaatatacacat | |
| | | ttaatcactggc | |
| | | (SEQ ID NO: 11) | |
| Cl90 | StdsbI-1 | Gactccatgggctca | NcoI |
| | | acggcaagtacctta | |
| | | tctatacca | |
| | | (SEQ ID NO: 12) | |
| Cl91 | StdsbI-2 | Cgtcggatcctcgtt | BamHI |
| | | cagtttcaaagaacg | |
| | | acgaata | |
| | | (SEQ ID NO: 13) | |
| Cl94 | VcdsbB-1 | Gactccatgggcatt | NcoI |
| | | tcaattgaaactgaa | |
| | | actaatcca | |
| | | (SEQ ID NO: 14) | |
| Cl95 | VcdsbB-2 | cgtcggatcctaaac | BamHI |
| | | agcagaaacaacaaa | |
| | | agtaa | |
| | | (SEQ ID NO: 15) | |
| Cl100 | FtdsbB-1 | Gactccatgggcaaa | NcoI |
| | | ctcagaaacacgcta | |
| | | aagcagc | |
| | | (SEQ ID NO: 16) | |
| Cl101 | FtdsbB-2 | Cgtcggatccagttt | BamHI |
| | | cttttgcttgagttt | |
| | | attttttgtttaa | |
| | | (SEQ ID NO: 17) | |
| Cl96 | HidsbB-1 | Gactccatgggcctg | NcoI |
| | | gctattgaatttatt | |
| | | ttaccag | |
| | | (SEQ ID NO: 18) | |
| Cl97 | HidsbB-2 | Cgtcggatcctagca | BamHI |
| | | aaatcagttaccgtt | |
| | | gaata | |
| | | (SEQ ID NO: 19) | |
| Cl65 | Ins | Attccggggatccgt | — |
| | ΔdsbB-1 | cgacctgcagttcga | |
| | | agttcctattctcat | |
| | | ctaaagtatatatga | |
| | | gtaaacttggt | |
| | | (SEQ ID NO: 20) | |
| Cl66 | Ins | Ttagtgtaggctgga | — |
| | ΔdsbB-2 | gctgcttcgaagttc | |
| | | ctatactttctaccg | |
| | | ggagctgcatgtgtc | |
| | | agaggttttc | |
| | | (SEQ ID NO: 21) | |

### The In Vivo Assay.

Our high throughput screen (HTS) for compounds that prevent the virulence of gram-negative bacteria is based on the ability to screen for inhibition of an enzyme (DsbB) that is not essential for bacterial growth, but is essential for virulence. It is a target-based as well as a cell-based assay in that we can readily determine in the screen itself whether the target is very likely the protein being inhibited by a compound detected in the HTS.

Since inhibition of DsbB by the candidate compounds does not inhibit bacterial growth, there can be no assessment of an IC50 for growth inhibition by a compound. Instead, we can determine an IC50 for the inhibition in vivo 1) of oxidation of DsbA, the substrate of DsbB, 2) of oxidation of a substrate of DsbA or 3) manifestation of a phenotype that is dependent on the activity of a disulfide-bonded protein (e.g. β-galactosidase activity). For any one compound, the three kinds of IC50s (1, 2 and 3) obtained can be quite different. As a result, the main useful feature of presenting IC50s by any one of these criteria is that we can rank the compounds in their degree of potency in inhibiting DsbB. Thus, we have chosen the most quantitative in vivo assay we have for the effect of inhibitors—the ability of the DsbB-DsbA pathway to inactivate the disulfide-bonded β-galactosidase. This in vivo assay for inhibition of DsbB is done in growing E. coli cells expressing a β-galactosidase that is sensitive to disulfide bond formation (Bardwell, et al., Cell. 67:581-589 (1991)). The inhibition of DsbB by our compounds is reflected in this assay by a positive signal, the restoration of β-galactosidase activity. For this reason, we have chosen to define the EC50 as the concentration of inhibitor that restores 50% of the maximal β-galactosidase activity which is exhibited by a strain completely defective in DsbB.

The E. coli strain (DHB7935) with which this assay is done expresses from its chromosome a gene fusion that encodes the MalF-β-galactosidase disulfide-sensitive enzyme. To make this strain more sensitive for the assay, we genetically altered DHB7935 to express DsbB at a level lower than the wild-type E. coli strain. This change was made because the high levels of DsbB produced in the wild-type strain make it difficult to measure the DsbB inhibition in terms of β-galactosidase activity in cultures grown in liquid (see below SAR section).

Despite the remarks above about the differing ways in which one could obtain IC50s for inhibition of DsbB, the limited data we have on IC50s as assessed by the oxidation of DsbA by DsbB are consistent with the IC50s we obtain with the β-galactosidase assays. That is, the two assays give IC50 numbers (nM or μM concentrations) that are nearly identical in the case of each of the two compounds (16 and 16.6) for which we have measured the oxidation state of DsbA in vivo. (Bardwell, et al. Cell. 67:581-589 (1991)).

### The In Vitro Assay.

In this assay, we determine the concentration of a compound necessary to reduce the activity of purified DsbB enzyme by 50%. DsbB uses ubiquinone as a co-factor for the generation of disulfide bonds. DsbB oxidizes DsbA and reduces ubiquinone. When it is in the oxidized state, ubiquinone absorbs strongly at 275 nm but it has a diminished absorbance when it is in the reduced state at the same wavelength. Therefore, DsbB activity can be measured by the reduction of ubiquinone and this is assessed by following the decrease of absorbance of ubiquinone at 275 nm. The assay uses 20 μM purified and DTT-reduced DsbA, 20 uM UbQ-5, and 2 nM purified DsbB at pH6.0.

### Agar Screening Plate Preparation.

A Matrix Wellmate (Thermo Scientific) fitted with a small-bore tubing cartridge was used to dispense 50-μL aliquots of hot agar medium (M63 medium containing 0.2% glucose and 0.9% agar, supplemented with kanamycin (40 μg/mL), ampicillin (50 μg/mL), IPTG (1 mM), and X-Gal (120 μg/mL)) to 384-well tissue culture-treated plates (BD Falcon #353289). In order to prevent agar solidification in the Wellmate tubing (at too low temperatures) or inactivation of the antibiotics and X-Gal (at too high temperatures), the medium was maintained at 57° C. by a water bath throughout the pouring process. In addition, the Wellmate tubing was pre-warmed by washing with sterile hot water immediately prior to loading the agar medium, and the plates were poured as quickly as possible. By using these techniques, we were able to prepare up to 80 uniform 384-well screening plates at a time. After the agar solidified, the plates were stored overnight in a sealed container at 4° C.

### High-Throughput Chemical Screen.

Most of the compound collections were supplied by the Institute of Chemistry and Cell Biology (ICCB) at Harvard Medical School. The initial screen included 50,374 compounds from several commercial small molecule libraries (Asinex 1, ChemBridge 3, ChemDiv 4, Life Chemicals 1, and Enamine 2) and small libraries of known bioactive molecules and natural products. In addition, a 1113-compound library of M. tuberculosis H37Rv growth inhibitors provided by the National Institute of Allergy and Infectious Diseases was assayed. Aliquots (100 nL) of library compounds (typically 5 mg/mL in DMSO) were transferred to the agar surface in each well of the screening plates by pin transfer. Because many of the experimental compounds are not water-soluble, it was important for them to be placed on top of the medium rather than injected deep within the agar, where contact with bacteria would be extremely limited. Therefore, the agar concentration was optimized (0.9%) to balance this requirement with the need for the medium to remain in the liquid state while pouring the plates.

Overnight E. coli cultures grown in minimal media were diluted to OD600=0.05 with M63 medium containing glucose (0.2%), kanamycin (40 μg/mL), ampicillin (50 μg/mL), and IPTG (1 mM). The diluted bacteria were added to each well in 10-μL aliquots with a Matrix Wellmate dispenser. It was necessary to increase the volume of bacteria from 5 μL (at OD600=0.1) to 10 μL (at OD600=0.05) to ensure that the concave agar well surfaces were completely covered. Positive (a strain lacking DsbB) and negative (no inhibitor) controls were included on each plate. The plates were sealed with breathable sealing film (Axygen BF-400) and incubated for three days at 30° C. in a humidified box. To enhance the blue color of the X-Gal hydrolysis product, the plates were incubated for 12-24 h at 4° C.

The compounds identified as inhibitors in the first round of screening were retested in a cherry-pick assay. The experiment was identical to the initial screen, except that the compounds were added to aliquots of bacteria with PocketTips (Thermo Scientific) rather than directly to the plates, and then the bacteria-compound mixtures were transferred to 384-well agar plates.

We point out at this juncture that there is a feature to our screen that we did not perceive initially which 1) makes our screen more sensitive than we realized and 2) does not allow us to estimate real IC50s or MICs with the 384-well plates containing agar media. This property of our screen results from the procedure in which aliquots of solutions of potential inhibitors are dropped onto solidified agar media in the wells. We believe that any “calculated concentrations” we might arrive at in such experiments are unlikely to reflect real concentrations in the mini-agar media in the wells. While we can calculate a perhaps false concentration (a minimum likely concentration) based on the amount of compound and the volume of the mini-agar media in the well, the real concentration is likely to be considerably higher at the top of the agar media where the bacteria are growing, depending on the solubility of the compound. Thus, these “apparent” MICs clearly did not reflect real MICs and conflicted substantially with numbers obtained from inhibition studies in liquid media. However, the actual higher concentration of compounds on the agar media does facilitate the detection of inhibitors, since strong inhibition is necessary for manifestation of an easily observable blue color generated by restoration of high levels of β-galactosidase activity.

### Compound Resupply.

Compounds 1, 2, 4, 5, 12, 13, 15, 16, 17, 19, 20, 21, 23, 16.3, 16.4, 16.20 and 16.21 were purchased from ChemBridge (San Diego, Calif.); 3 from ChemDiv (San Diego, Calif.); 6, 7, and 18 from Asinex Ltd. (Moscow, Russia); 8 from Sequoia Research Products Ltd. (Pangbourne, UK); 14 from Key Organics Ltd. (Camelford, UK); 22 from Sigma Aldrich (St. Louis, Mo.); 16.1 and 16.2 from AK Scientific (Union City, Calif.); 16.5, 16.6, 16.7, 16.8 and 16.23 from Ambinter (Orleans, France); 16.24 from Ryan Scientific (Mt. Pleasant, S.C.); and compounds 16.9, 16.10, 16.11, 16.12, 16.13, 16.14, 16.15, 16.16, 16.17, 16.18, 16.19 and 16.22 from Enamine (Ukraine). Larger quantities of compounds 2, 5, and 6 were obtained by custom synthesis (Aberjona Laboratories, Inc.; Beverly, Mass.) and of compound 16.6 were purchased from Enamine (Ukraine). All purchased compounds were analyzed by mass spectrometry to verify the molecular weights and to estimate purity (ICCB). In addition, 1H NMR spectra were collected for compounds 5 and 6 (ICCB).

M. tuberculosis H37Rv Growth Inhibition.

The bacteria were grown to stationary phase (OD=2.0) and diluted to OD=0.003. Chemical compounds were dissolved in growth medium and subsequent serial dilutions (0.12-125 μg/mL) were performed in 96-well plates. A bacterial inoculum (50 μL) was added to each well, yielding a final volume of 100 μL/well. This experiment was repeated with 7H9 medium, 7H9 medium supplemented with OADC, and Sauton's medium. The plates were sealed with breathable film and incubated in a shaker at 37° C. After five days, an aliquot of Alamar Blue reagent (10 μL Biosource) was added to each well, and the plates were incubated for 24 h at 37° C. The MIC was defined as the lowest drug concentration that prevented a color change from blue to pink.

The color of compounds 2 and 6 interfered with the Alamar Blue assay, so MIC values were obtained by performing serial dilutions in 24-well plates and using OD600 as a measure of growth Compound 6 presented additional challenges. Inclusion of this inhibitor delayed growth in all wells (including no drug controls) of the plates, which were covered with breathable sealing film. It seems that compound 6 releases, or causes to be released, a volatile chemical that kills cells or affects the media, thereby killing cells. To determine accurate MIC values for compound 6, individual inkwells were used in place of the 24-well plates.

### Preparation of Mouse Liver Microsomes.

Mouse hepatic microsomes were used as a source of VKOR. Microsomes were obtained from mouse liver by homogenization in PBS/20% glycerol/protease inhibitor cocktail (PIC) (Calbiochem, 1× final concentration) using a Potter tissue grinder with an attached power unit (Con-Torque/Eberbach). Mouse liver (10 g) was homogenized with ten strokes of the tissue grinder 4 times with cooling on ice after each 10 strokes. The sample was centrifuged at 10,780×g for 10 min at 4° C. The supernatant was collected and the remaining pellet was subjected to another cycle of homogenization as before. After two more cycles of homogenization the four supernatants were pooled and subjected to centrifugation at 38,000×g for 1 h at 4° C. The pellet from this centrifugation was resuspended in PBS/20% glycerol/PIC/0.2% phosphatidycholine/0.5% CHAPS and sonicated twice with a Microson XL sonicator (Misonix) at power level 4 with cooling on ice after each sonication. The sample was centrifuged at 38000×g for 1 h at 4° C. The supernatant from this centrifugation containing the solubilized liver microsomes was stored at −80 C.

### Preparation of Vitamin K Epoxide.

Vitamin KI (20 mg) was dissolved in 3 mL of isopropanol/hexane (2:1 v/v) containing 100 μL of 0.5 M NaOH in 0.2 M Na₂CO₃ and 300 μL of 30% H₂O₂. The mixture was protected from light and incubated overnight at 37° C. Water was added until the two phases separated. The upper hexane layer was collected and evaporated to dryness under a stream of nitrogen at 50° C. The dry residue was suspended in methanol and vitamin K epoxide was purified by HPLC on a C18 column (Vydac). The concentration of the purified vitamin K epoxide was measured at an absorbance of 226 nm using the known extinction coefficient of vitamin K epoxide.

### Assay for VKOR Enzymatic Activity.

Solubilized mouse liver microsomes (20 μL) were added to 180 μL of buffer (25 mM N-[Tris(hydroxymethyl)methyl]-3-aminopropanesulfonic acid, pH 8.6 in 150 mM NaCl/30% glycerol. When inhibitors were used they were added at the indicated concentrations to the solubilized microsomes in buffer and the mixture incubated for 10 min at 4° C. The substrate, 4 μL of 12 mM vitamin K epoxide in isopropanol, was added to the microsomes and 5 μL of 200 mM DTT was added to start the reaction. The reaction mixture was incubated for 24 h at room temperature protected from light. The reaction was stopped by adding 500 μL of a mixture of 0.05 M AgNO₃ in isopropanol (5:9 v/v). The mixture was vortexed for 1 min and centrifuged to separate the phases. The upper organic phase (400 μL) was transferred to a brown vial and dried with a gentle stream of nitrogen. The dried sample was dissolved in acetonitrile:isopropanol:water (100:7:2 v/v) which also served as the mobile phase for HPLC. The concentration of vitamin K epoxide was determined by HPLC analysis on a C18 column (Vydac) and the amount of vitamin K epoxide converted to vitamin K calculated using a known concentration of vitamin K epoxide as a standard.

### Insulin Reductase Activity Assay.

The activity of thiol isomerases was tested using the insulin reductase assay. The catalyzed reduction of insulin was measured in the presence of DTT. During reduction a white precipitate forms. The rate of precipitation was measured by absorption at 650 nm. The assay was performed in 384-well plates at 25° C. The final reaction volume was 30 μl. The reaction mixture contained 0.3 mM DTT, 0.4 μM insulin, 2 mM EDTA dissolved in 100 mM potassium phosphate pH 7.4 and the enzyme to be tested. His-tagged thioredoxin-1 (R & D Sytems Inc., Minneapolis, Minn.) and his-tagged PDI (Prospec, East Brunswick, N.J.) were used at a final concentration of 1 μM. His-tagged ERp72 (Enzo, Farmingdale, N.Y.), his-tagged ERp57 (AbCam, Cambridge, Mass.) and his-tagged ERp5 (Passam F., Furie, B. Furie B.C.) were used at a final concentration of 0.8 μM. Inhibitor compounds at the indicated concentrations or buffer as control were included in the reaction mixture. The reaction was initiated by the addition of DTT.

### Analysis of Structure-Activity Relationship (SARI.

A substructure search of compounds with a pyridazinone core was performed to detect molecules similar to compound 16 (DsbB inhibitor) among the ICCB-libraries of compounds tested and not tested in the agar screening. The obtained list of similar molecules was analyzed by looking for compounds that did and did not turn the bacteria blue in the DsbB screening and were categorized then as inhibitors or non-inhibitors respectively. Based on that information, compounds with substitutions at position 6 of the pyridazinone were discarded since it was detrimental for the inhibitory activity of the compound. Compounds that did have a single change (compared to compound 16) either at position 2, 4 or 5 were selected as candidates to test. In order to determine if those candidates were commercially available, a substructure search for the pyridazinone was done using SciFinder software (American Chemical Society). 24 compounds out of the 57 commercially available candidates were purchased (see Compound resupply section) and tested in liquid media against E. coli DHB7935 strain (see Materials and Methods), which expresses dsbB gene from a low activity Trc promoter, making it more sensitive to weaker inhibitors. This strain allowed us to more easily rank the compounds from potent to weak inhibitors. The EC50s in this assay for each compound were determined by quantifying the β-galactosidase activity of DHB7935 in the presence of different concentrations of compound. The EC50s were defined as the concentration of compound in which the strain reaches 50% β-galactosidase activity compared to the 100% obtained in ΔdsbB strain (HK325, positive control). To measure β-galactosidase activity, the velocity of hydrolysis of o-nitrophenyl-β-galactoside (ONPG, Sigma) was determined. The assay was done in a flat bottom 96-well plate (Thermo Scientific) as described previously [Thibodeau S. Fang R. and Joung K. High throughput b-galactosidase assay for bacterial cell-based reporter systems. BioTechniques 36(3):410-414. (2004)]. Briefly, DHB7935 cells were inoculated to an OD₆₀₀ of 0.01 in 200 μl of M63 with 0.2% glucose as a carbon source, 0.2% maltose to induce the expression of MalF-LacZ1022 fusion and with serial dilutions of inhibitor. The cells were incubated for 12 hours at 30° C., 80% humidity and 900 rpms in an orbital shaker (Multitron, ATR). 100 μl of cells were lysed using 10 μl of PopCulture reagent (Novagen) and incubated with 90 μl of 4 mg/ml ONPG at 28° C. in a microplate reader (VERSAmax). The OD₄₂₀ was measured every minute during 1 hour to follow the kinetics of ONPG hydrolysis and the velocity of the reaction was calculated by SoftMax®Pro software (Molecular Devices, LLC). Miller Units were determined using 1.81 (CFI), 2.45 (CF2) and 3.05 (CF3) as constants and finally the EC50 was calculated by GraphPad Prism Software with non-linear log dose-response normalized curve using 4 parameters. The experiments were done by at least four replicates. In order to observe the difference in activity and have more meaningful idea of the inhibitor potency, all the EC50 values were compared to the EC50 of compound 16 and the EC50 ratio was calculated for each dividing the EC50 of compound 16 between the EC50 of the compound (see Table 5).

### In Vivo Chemical Alkylation.

The in vivo chemical alkylation procedure was done as described previously (Chng S et al. Science 337, 1665-1668 (2012)). Briefly, a 1 mL culture was grown to OD₆₀₀≈0.5 in M63 glucose minimal medium with appropriate concentrations of drug (compound 16 or compound 16.6). A 500 μl culture aliquot was then transferred to a 1.5 ml tube and precipitated with 50 μl of trichloroacetic acid (TCA, 100%, Mallinckrodt Baker). The mixture was incubated on ice for 10 min and precipitated proteins were pelleted at 18 000 g for 10 min. The protein pellet was washed with 600 μl ice-cold acetone and incubated on ice for 10 min. The protein was re-pelleted by centrifugation at 18 000 g for 3 min and left to air-dry at room temperature. The TCA-precipitated proteins were either directly subjected to alkylation or first reduced before alkylation. For reduction, proteins were incubated in 100 μl 100 mM Tris HCl, pH 8.0 containing 0.1% SDS and 100 mM dithiothreitol (DTT, Invitrogen) for 30 min at room temperature. M63 (500 μl) medium was added and the reduced proteins were re-precipitated with TCA and further washed with acetone. Precipitated proteins (reduced or not) were then solubilized in 50 μl of 100 mM Tris.HCl, pH 6.8 containing 1% SDS and 5 mM 4-acetamido-4′-maleimidylstilbene-2,2′-disulfonic acid (AMS). The mixture was mixed in a water bath sonicator for 10 min and incubated for 1 hr at 37° C. The AMS was quenched with 100 mM DTT. Non-reducing 3×-SDS sample buffer was then added and 2 μl of sample was applied to SDS-PAGE directly. Tris-HCl polyacrylamide (12%) gels were used (running conditions: 150 V for 1 h). The proteins were transferred onto PVDF membranes and immunoblotted with α-DsbA antibody (Bardwell et al., 1991).

Testing DsbB Inhibitors Against Other Gram-Negative DsbB Enzymes Expressed in E. coli.

In order to test E. coli strains expressing the dsbB genes from other organisms, the 384-well plates were prepared in the same way as in the high throughput screening only differing in that 0.2% maltose was included in the media to induce the expression of the MalF-LacZ102 fusion. In order to have effective complementation of A. baumannii DsbB and P. aeruginosa DsbH (strains CL377 and CL378 respectively), 50 μM of IPTG (Isopropyl β-D-thiogalactoside, Promega) was added to the agar media. For S. typhimurium DsbI (strain CL368), 75 μM of IPTG was required. For F. tularensis DsbB (strain CL370) 2 mM of IPTG was added to the media. For P. aeruginosa DsbB, K. pneumoniae DsbB, S. typhimurium DsbB, V. cholerae DsbB and H. influenzae DsbB (strains CL315, CL320, CL369, CL373 and CL371, respectively) basal expression levels (no IPTG) were sufficient for effective complementation of dsbB mutant strain.

Two compound plates (Corning 384-well storage plates, polypropylene round bottom) were prepared with the entire collection of 30 compounds purchased as a result of the SAR. Dilutions of the compounds were dispensed in the 384-well plate ranging from 30 mM to 0.6 μM. 100 nL aliquot of the compounds were transferred to solidified-agar plates by pin transfer (EPSON compound transfer robot) in order to have a final concentration ranging from 50 μM to 0.001 μM of compound, except compounds 16.7 and 16.8 which highest concentration started at 28.9 μM and 26.4 μM, respectively. Then, 10 μL of the bacterial cultures at 0.05 of OD₆₀₀ were added to the agar plates with a Matrix Wellmate (Thermo Scientific). Plates were sealed with breathable sealing film (Axygen BF-400) and incubated at 30° C. for one day in humidified boxes and 1 day at 4° C. to analyze the results. CL379 was included as a positive control in each plate.

The minimal concentration of each compound that caused the bacteria to turn light blue was registered for all of the strains expressing dsbB genes from pathogenic bacteria. These concentrations were used to rank the compounds from strong to weak inhibitors by dividing the concentration between the lowest minimal concentration observed for that particular strain expressing DsbB. In this way we obtained the ranking ratio of three independent experiments and the average was calculated and plotted in a color-coded table using Excell, shown here adapted into grayscale (Table 6).

### Example 2

### Inhibition of Purified EcDsbB

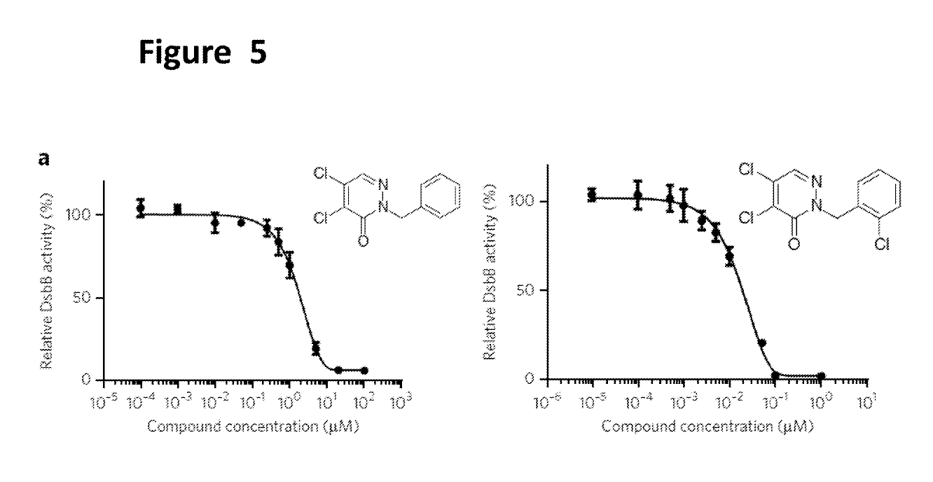

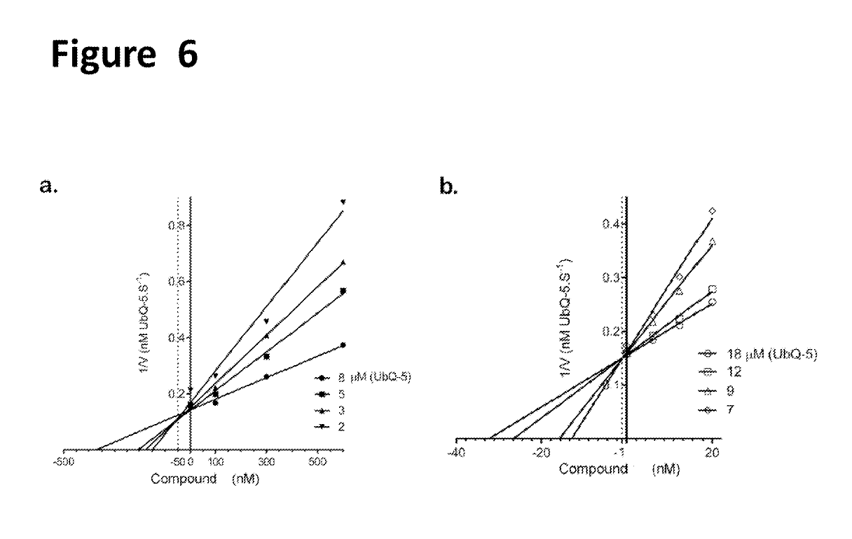

We tested several compounds for their inhibitory effect on EcDsbB-mediated ubiquinone reduction using purified enzymes. In this assay, reduced DsbA provides the source of electrons that are used by EcDsbB to reduce ubiquinone-5. The compounds show dose-dependent inhibition of EcDsbB with half-maximal inhibitory concentrations (IC₅₀) in the low-μM range. The lowest IC₅₀, 1.7 μM, was that of compound 16 (FIG. 5). Enzyme kinetics analysis of compound 16 revealed a Kᵢ of 46±20 nM (the K_{M} for ubiquinone-5 is 1.03+/−0.12 μM, FIG. 6).

### Mechanism of EcDsbB Inhibition by Compound 16.6

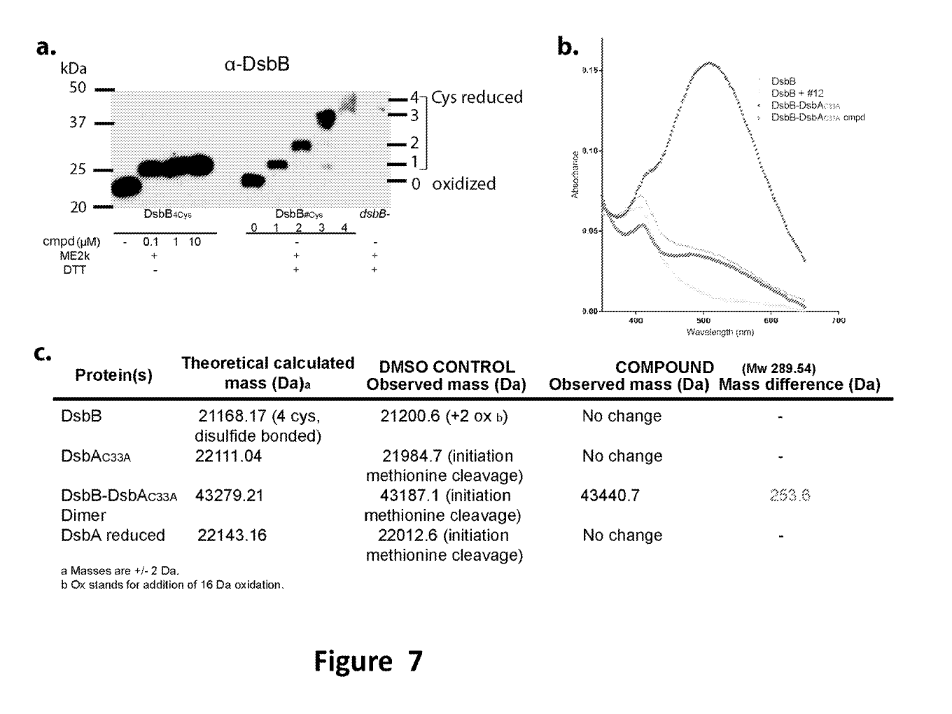

Among the compounds discussed above, we found two EcDsbB inhibitors (16.2 and 16.6) that showed 10- and 23-fold more inhibitory activity than 16, respectively. Compound 16.6 has a Kᵢ of 0.8±0.1 nM (IC₅₀ of 18.85 nM) in the in vitro assay (FIG. 5 and FIG. 6) and an IC₅₀ of 0.9±0.5 μM in inhibiting DsbA oxidation in aerobically growing cells. Additionally, when we probed the in vivo redox state of the cysteines of EcDsbB with the cysteine alkylating agent maleimide PEG-2k (ME2k) after treatment of cells with compound 16.6, EcDsbB showed only one ME2k modification (FIG. 7A). This indicates that of the four essential EcDsbB cysteines (the two nonessential cysteines are mutated to alanine and valine, respectively), two are in the disulfide state, one is in the reduced state (labeled by ME2k) and the fourth is unavailable to react with ME2k.

Further, we noticed that the slightly pinkish color of purified DsbB changes to yellow when DsbB is treated with compound 16.6. The pink color represents a small population of DsbB that is in the charge-transfer complex state with ubiquinone, which absorbs strongly at 500 nm. When DsbAC33A is used as a substrate for DsbB, it forms a stable mixed disulfide complex in which Cys44 is trapped in the charge-transfer complex state. Likewise, when the compound was added to the DsbB-DsbA_{C33A} dimer, its characteristic color turned yellow (FIG. 7B). This was not due to the dissociation of the dimer, since nonreducing SDS-PAGE showed the complex to be intact. Moreover, when compound 16.6 was added to DsbB before the addition of DsbA_{C33A}, the pink color quickly developed but did not persist. These results suggested that the compound influences the interaction between Cys44 of DsbB and ubiquinone.

To determine whether the interaction between compound 16.6 and the DsbB-DsbA_{C33A} is due to a covalent bond, we performed ion-trap mass spectrometry, which revealed an adduct of 253.6 Da with the DsbB-DsbA_{C33A}, dimer (FIG. 7B). Since the theoretical molecular weight of compound 16.6 is 289.54 Da, the 35.9-Da mass loss may represent a leaving chloride ion. This mass adduct was not observed when either DsbB (oxidized), DsbA (reduced) or DsbAC33A, alone was treated with the compound (FIG. 7C). These data reveal a covalent modification of DsbB by compound 16.6 that occurs after the formation of the charge-transfer complex during ubiquinone reduction. Consistent with this expectation, high-resolution tandem mass spectrometry of chymotrypsin-digested DsbB-DsbA_{C33A} complex treated with the compound shows that Cys44 of DsbB has an adduct of 252.995 Da.

### Obtaining More Effective EcDsbB Inhibitors

A medicinal chemistry approach was taken to obtain of pyridazinone inhibitors. A substructure search of compounds with a pyridazinone core was performed to detect molecules similar to compound 16 (DsbB inhibitor). Initially, 24 compounds out of the 57 commercially available candidates were purchased and later 20 more compounds were synthesized (Sundia MediTech Company) and tested in liquid media against E. coli DHB7935 strain, which expresses dsbB gene from a weaken Trc promoter, making it more sensitive to weak inhibitors. This strain allowed us to easily rank the compounds. The Relative Inhibitory Concentration 50 (RIC50) for each compound was determined by quantifying the β-galactosidase activity of DHB7935 strain in the presence of different concentrations of compound. The RIC50 was defined as the concentration of compound in which the strain reaches 50% β-galactosidase activity compared to the 100% obtained in ΔdsbB mutant strain (DHB7936). To measure β-galactosidase activity, the velocity of hydrolysis of o-nitrophenyl-β-galactoside (ONPG, Sigma) was determined. The assay was done in a flat bottom 96-well plate (Thermo Scientific) as described previously. Briefly, DHB7935 cells were inoculated to an OD₆₀₀ of 0.01 in 200 μL of M63 with 0.2% glucose as a carbon source, 0.2% maltose to induce the expression of β-Gal^{dbs} and with serial dilutions of inhibitor. The cells were incubated for 12 hours at 30° C., 80% humidity and 900 rpms in an orbital shaker (Multitron, ATR). 100 μL of cells were lysed using 10 μL of PopCulture reagent (Novagen) with 400 U/mL lyzosyme and incubated with 90 μL of 4 mg/mL ONPG at 28° C. in a microplate reader (VERSAmax). The OD₄₂₀ was measured every minute during 1 hour to follow the kinetics of ONPG hydrolysis and the velocity of the reaction was calculated by SoftMax®Pro software (Molecular Devices, LLC). Miller Units were determined using 1.81 (CF1), 2.45 (CF2) and 3.05 (CF3) as constants and the relative β-galactosidase activity was calculated normalizing to the full activity obtained for the dsbB mutant (100%). Finally, the RIC50 was calculated by GraphPad Prism Software (La Jolla Calif., USA) with non-linear log dose-response normalized curve using 4 parameters. The RIC50 values and 95% confidence intervals were obtained using data of at least three independent experiments. Results for each relevant compound are shown below in Table 9.

TABLE 9

| Com- | | RIC50 | IC50 |
|---|---|---|---|
| pound # | Structure | (μM) | (μM) |
| C16.27 | 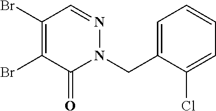 [SMILES: O=c1c(Br)c(Br)cnn1Cc1ccccc1Cl] | 0.025 | |
| C16.6 | 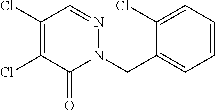 [SMILES: O=c1c(Cl)c(Cl)cnn1Cc1ccccc1Cl] | 0.16 | 0.01885 |
| C16.43 | 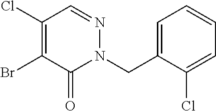 [SMILES: O=c1c(Br)c(Cl)cnn1Cc1ccccc1Cl] | 0.29 | |
| C16.44 | 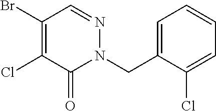 [SMILES: O=c1c(Cl)c(Br)cnn1Cc1ccccc1Cl] | 0.32 | |
| C16.12 | 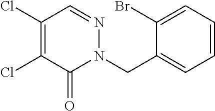 [SMILES: O=c1c(Cl)c(Cl)cnn1Cc1ccccc1Br] | 0.47 | |
| C16.20 | 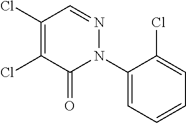 [SMILES: O=c1c(Cl)c(Cl)cnn1-c1ccccc1Cl] | 1.34 | |
| C16.42 | 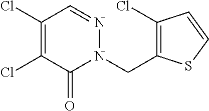 [SMILES: O=c1c(Cl)c(Cl)cnn1Cc1sccc1Cl] | 1.45 | |
| C16.2 | 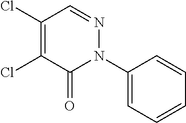 [SMILES: O=c1c(Cl)c(Cl)cnn1-c1ccccc1] | 2.04 | 2.34 |
| C16.35 | 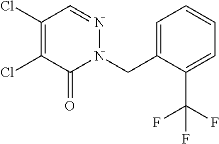 [SMILES: O=c1c(Cl)c(Cl)cnn1Cc1ccccc1C(F)(F)F] | 2.81 | |
| C16.23 | 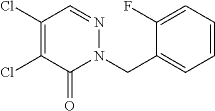 [SMILES: O=c1c(Cl)c(Cl)cnn1Cc1ccccc1F] | 3.09 | |
| C16.13 | 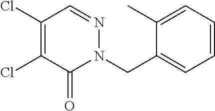 [SMILES: Cc1ccccc1Cn1ncc(Cl)c(Cl)c1=O] | 3.56 | |
| C16 | 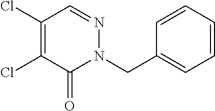 [SMILES: O=c1c(Cl)c(Cl)cnn1Cc1ccccc1] | 5.1 | 1.855 |
| C16.36 | 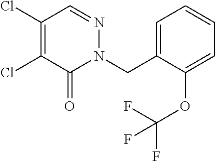 [SMILES: O=c1c(Cl)c(Cl)cnn1Cc1ccccc1OC(F)(F)F] | 7.12 | |
| C16.25 | 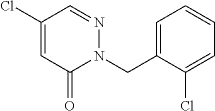 [SMILES: O=c1cc(Cl)cnn1Cc1ccccc1Cl] | 8.37 | |
| C16.40 | 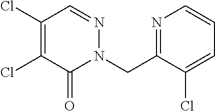 [SMILES: O=c1c(Cl)c(Cl)cnn1Cc1ncccc1Cl] | 10.84 | |
| C16.17 | 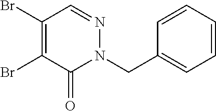 [SMILES: O=c1c(Br)c(Br)cnn1Cc1ccccc1] | 13.21 | |
| C16.14 | 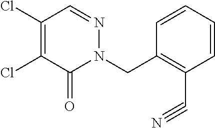 [SMILES: N#Cc1ccccc1Cn1ncc(Cl)c(Cl)c1=O] | 13.21 | |
| C16.24 | 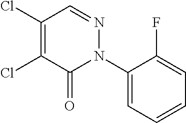 [SMILES: O=c1c(Cl)c(Cl)cnn1-c1ccccc1F] | 13.68 | |
| C16.4 | 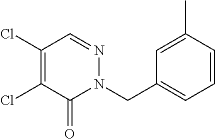 [SMILES: Cc1cccc(Cn2ncc(Cl)c(Cl)c2=O)c1] | 13.91 | 1.275 |
| C16.39 | 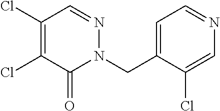 [SMILES: O=c1c(Cl)c(Cl)cnn1Cc1ccncc1Cl] | 15.18 | |
| C16.22 | 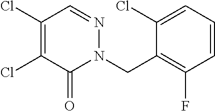 [SMILES: O=c1c(Cl)c(Cl)cnn1Cc1c(F)cccc1Cl] | 18.15 | |
| C16.26 | 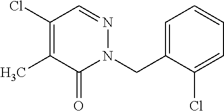 [SMILES: Cc1c(Cl)cnn(Cc2ccccc2Cl)c1=O] | 18.7 | |
| C14 | 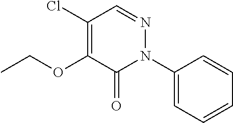 [SMILES: CCOc1c(Cl)cnn(-c2ccccc2)c1=O] | 25.63 | 6.57 |
| C15 | 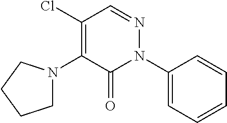 [SMILES: O=c1c(N2CCCC2)c(Cl)cnn1-c1ccccc1] | 28.12 | 8.24 |
| C13 | 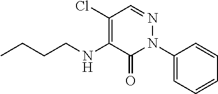 [SMILES: CCCCNc1c(Cl)cnn(-c2ccccc2)c1=O] | 30.43 | 8.12 |
| C16.41 | 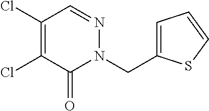 [SMILES: O=c1c(Cl)c(Cl)cnn1Cc1cccs1] | 33.14 | |
| C16.8 | 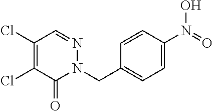 | 38.67 | |
| C12 | 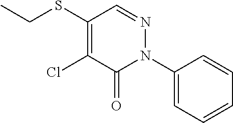 [SMILES: CCSc1cnn(-c2ccccc2)c(=O)c1Cl] | 61.09 | 11.54 |
| C17 | 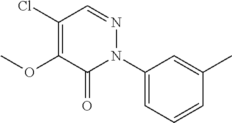 | 72.84 | 5.42 |
| C16.16 | 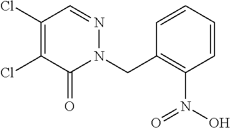 | 90.47 | |
| C16.11 | 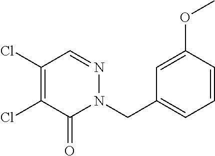 [SMILES: COc1cccc(Cn2ncc(Cl)c(Cl)c2=O)c1] | 231.8 | |
| C16.9 | 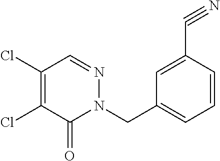 [SMILES: N#Cc1cccc(Cn2ncc(Cl)c(Cl)c2=O)c1] | 509.2 | |
| C16.37 | 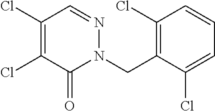 [SMILES: O=c1c(Cl)c(Cl)cnn1Cc1c(Cl)cccc1Cl] | 6,570 | |

### Testing DsbB Inhibitors Against Other Gram-Negative DsbB Enzymes Expressed in E. Coli

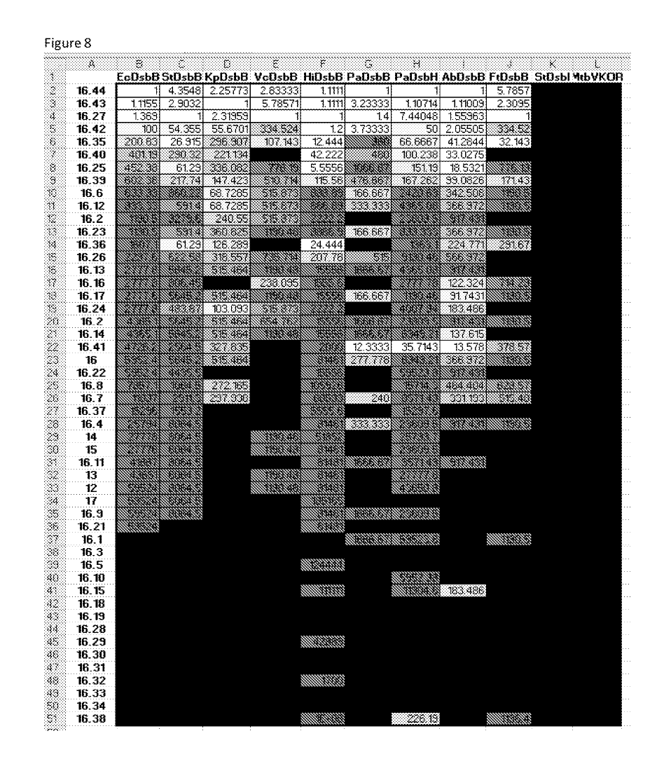

We further tested the EcDsbB inhibitors for their ability to inhibit DsbBs from other Gram-negative pathogens when expressed in E. coli. We cloned the dsbB genes from Acinetobacter baumanni, Klebsiella pneumonia, Vibrio cholerae, Hemophilus influenza, Francisella tularensis, two dsbB homologs from Pseudomonas aeruginosa (dsbB and dsbli) and two dsbB homologs from Salmonella typhimurium (dsbB and dsbl) under the control of an IPTG-inducible promoter. All DsbB homologs complemented the dsbB-null strain in maintaining β-Gal^{dbs} in the disulfide-bonded state, as indicated by the absence of blue color in agar growth assay. We then tested the complemented strains for their sensitivity to the collection of EcDsbB inhibitors and to the related non-inhibitory compounds. Several dilutions of these inhibitors were dropped onto the agar medium in 384-well plates with the complemented strains, thus allowing us to rank the different inhibitors in terms of their ability to inhibit each DsbB. Results are shown in FIG. 8.

For the most part, the compounds that did not inhibit EcDsbB also did not inhibit the DsbBs of the other Gram-negative bacteria, while at least one of those that inhibited EcDsbB also inhibited the other DsbBs. Interestingly, although one of the most effective inhibitors of several of the organisms was 16.6, in the agar assay other DsbBs were more effectively inhibited by other compounds of this group (FIG. 8). The only DsbB homolog that was not inhibited by any of these compounds, StDsbI, is an unusual homolog that appears to be involved in a specialized pathway of disulfide bond formation (Grimshaw et al., J. Mol. Bio. 380, 667-680 (2008); Lin et al., Microbiology 155, 4014-4024 (2009)). Although we can rank the inhibitors in terms of strong versus weak, it is not possible to compare the effectiveness of their action on different DsbBs without knowing for each DsbB the expression level and effectiveness of oxidizing EcDsbA.

Inhibition of DsbB Enzymes from Gram-Negative Bacteria Expressed in E. coli.

In order to test E. coli strains expressing the dsbB genes from other organisms, the 384-well plates were prepared in the same way as in the HTS only differing in that 0.2% maltose was included in the media to induce the expression of the β-Gal^{dbs}. In order to have effective complementation of A. baumannii DsbB and P. aeruginosa DsbH (strains CL377 and CL378 respectively), 50 μM of IPTG (Isopropyl β-D-thiogalactoside, Promega) was added to the agar media. For S. typhimurium DsbI (strain CL368), 75 μM of IPTG was required. For F. tularensis DsbB (strain CL370) 2 mM of IPTG was added to the media. For P. aeruginosa DsbB, K. pneumoniae DsbB, S. typhimurium DsbB, V. cholerae DsbB and H. influenzae DsbB (strains CL315, CL320, CL369, CL373 and CL371, respectively) basal expression levels (no IPTG) were sufficient for effective complementation of dsbB mutant strain.

Two compound plates (Corning 384-well storage plates, polypropylene round bottom) were prepared with the entire collection of 30 compounds purchased as a result of SAR (12 to 17 and 16.1 to 16.24) and four compound plates with the recent collection of 20 compounds obtained by custom synthesis (Sundia MediTech Company). Dilutions of the compounds were dispensed in the 384-well plate and 100 nL aliquot of the compounds were transferred to solidified-agar plates by pin transfer (EPSON compound transfer robot) in order to have a final concentration ranging from 50 μM to 0.001 μM for compounds 12-17 and 16.1-16.24 except compounds 16.7 and 16.8 which highest concentration started at 28.9 μM and 26.4 μM, respectively. Compounds 16.25 to 16.27, 16.35 to 16.37, 16.39 to 16.42 ranging from 30 μM to 0.00001 μM and for compounds 16.28 to 16.34, 16.38, 16.43 to 16.44 ranging from 100 μM to 0.00001 μM. Then, 10 μL of the bacterial cultures at 0.05 of OD₆₀₀ were added to the agar plates with a Matrix Wellmate (Thermo Scientific). Plates were sealed with breathable sealing film (Axygen BF-400) and incubated at 30° C. for one day in humidified boxes and one day at 4° C. to analyze the results. MER672 was used as EcDsbB expressing strain and CL379 was included as a positive control in each plate. The minimal concentration (MIC) of each compound that caused the bacteria to turn light blue was registered for all of the strains expressing dsbB genes from pathogenic bacteria. Since the MIC is related to the expression of each DsbB in E. coli we decided to use these MICs to rank the compounds from strong to weak inhibitors in each DsbB-expressing strain normalizing the data for each strain. Thus, we ranked compounds by dividing the MIC observed for each compound between the lowest MIC observed for that particular strain expressing DsbB. In this way we obtained the average of three independent experiments for the first collection of 30 compounds and one experiment for the last collection of 20 compounds. The ratios were plotted in a color-coded table, using conditional formatting (3-color rule) in Excel, the results of which is shown in grayscale in FIG. 8. (Black areas of the table have a value of “1E+08”).

Inhibition of DsbB Homologs in Pseudomonas aeruginosa PA14

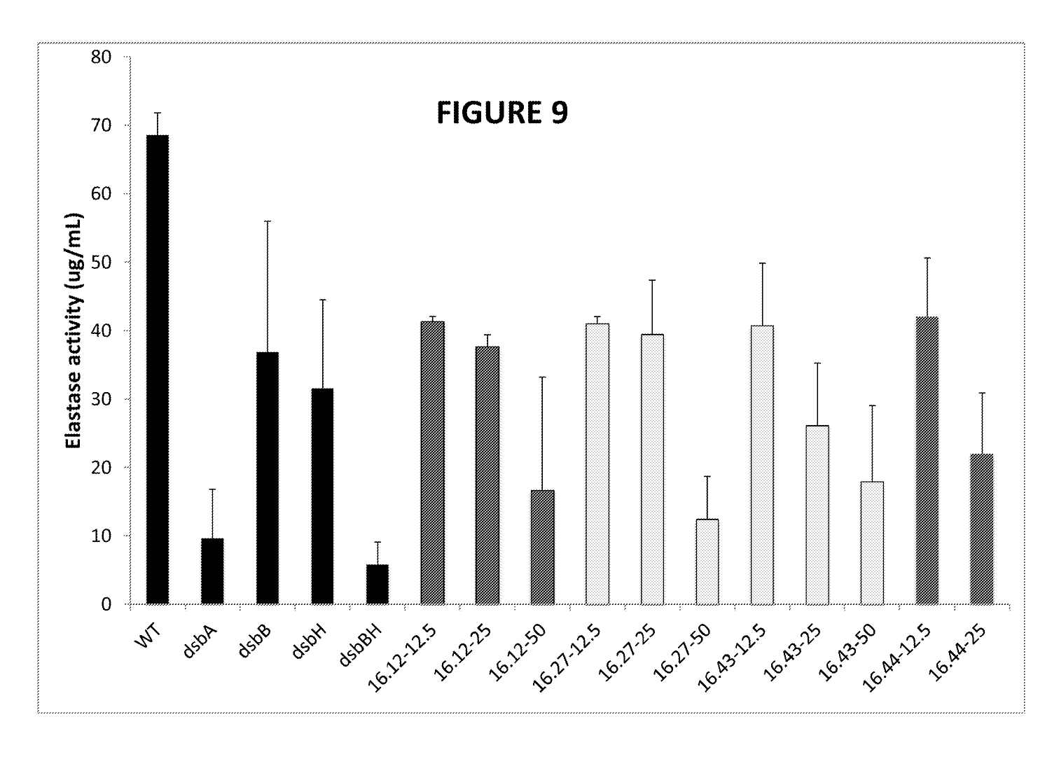

The opportunistic pathogen Pseudomonas aeruginosa secretes many proteins into the extracellular medium. For several proteins that are secreted via a type II mechanism, including elastase (encoded by lasB) of P. aeruginosa, it has been demonstrated that folding in the periplasm is essential for the subsequent translocation across the outer membrane to occur. This metalloprotease is produced as a preproprotein. The propeptide is essential for the folding of elastase in the periplasm, and this folding allows for further processing of the proenzyme by autoproteolytic cleavage. The propeptide of elastase does not contain any cysteines, whereas the mature polypeptide contains four of them, which together form two disulfide bonds in the folded enzyme. Both bonds are not localized in close proximity of the active center of the protein. One disulfide bond, between Cys-30 and Cys-57, is located in the N-terminal part of the mature enzyme and connects two β-strands. The other disulfide bond, between Cys-270 and Cys-297, is located close to the C terminus and connects two a-helices. One disulfide bond is formed in the proenzyme and is essential for subsequent autoproteolytic processing to occur. The other disulfide bond is formed only after autocatalytic processing and appeared to be required for the full proteolytic activity of the enzyme and contributes to its stability. It has also been shown that DsbA is involved in the formation of these disulfide bonds, hence mutations in DsbA are defective in elastase activity (Braun P. et al., 2001). Given that DsbB is necessary for the re-oxidation of DsbA, the knock out of the two DsbB homologs (DsbB and DsbH) present in P. aeruginosa is also defective in the formation of disulfide bonds and hence elastase activity. Elastase activities in the supernatant of P. aeruginosa cultures were quantified in the presence or absence of increasing amounts of several compounds (16.12, 16.27, 16.43, 16.44). The results are presented in FIG. 9.

Quantifying Elastase Activity in P. aeruginosa Cultures.

In order to quantify elastase activity in the supernatant of P. aeruginosa cultures, an overnight culture in LB medium was grown and diluted to and OD600 of 0.001 in M63 minimal medium. The cultures were incubated during 24 h in the presence (12.5, 25 and 50 μM) or absence of test compound at 37° C. and shaken at 200 rpms. Then, the supernatant of 1 mL of culture was obtained by centrifugation at 13,000 rpms and 10 μL of it was placed in a 96-well plate (black with clear bottom) and diluted with 85 μL of buffer A (50 mM Tris-HCl pH 7, 2.5 mM CaCl₂). The purified enzyme was used as a standard of the amount of activity:

| Enzyme | | Elastase (PaLasB 100 ng/μL) |
|---|---|---|
| concentration (ng) | Buffer A | (EMD Millipore, Calbiochem #324676) |
| Blank | 95 | — |
| 500 | 90 | 5 |
| 1000 | 85 | 10 |
| 2500 | 70 | 25 |
| 5000 | 45 | 50 |
| 7500 | 20 | 75 |

All reactions were started by the addition of 5 μL of 5 mM substrate (benzyloxycarbonyl Z-ala-gly-leu-ala-OH, Sigma #SC00185) to get a final concentration of 250 μM. Finally, the proteolysis was followed by measuring the fluorescence of the reaction during 1 h at λₑₓ 320 nm/λₑₘ 430 nm and at 37° C. The amount of elastase in the supernatant aliquots was calculated by interpolation of the velocities and the concentration of standards using hyperbola function (Prism, GraphPad). The mean and standard error were calculated from at least two independent experiments with two replicas each.

### Example 3

Synthesis of exemplary compounds is described below. Table 10 below correlates the compound numbering scheme used in Example 3 to the compound numbering scheme used in Table 1.

TABLE 10

| Table 1 | | Example 2 |
|---|---|---|
| number | Compound | number |
| 16.25 | 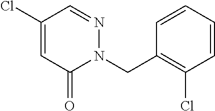 [SMILES: O=c1cc(Cl)cnn1Cc1ccccc1Cl] | G1-1 |
| 16.26 | 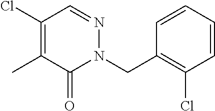 [SMILES: Cc1c(Cl)cnn(Cc2ccccc2Cl)c1=O] | G1-2 |
| 16.43 | 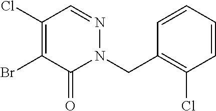 [SMILES: O=c1c(Br)c(Cl)cnn1Cc1ccccc1Cl] | G1-3 |
| 16.27 | 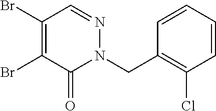 [SMILES: O=c1c(Br)c(Br)cnn1Cc1ccccc1Cl] | G1-4 |
| 16.44 | 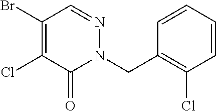 [SMILES: O=c1c(Cl)c(Br)cnn1Cc1ccccc1Cl] | G1-7 |
| 16.35 | 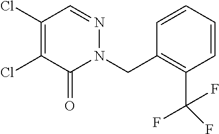 [SMILES: O=c1c(Cl)c(Cl)cnn1Cc1ccccc1C(F)(F)F] | G2-1 |
| 16.36 | 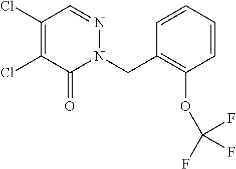 [SMILES: O=c1c(Cl)c(Cl)cnn1Cc1ccccc1OC(F)(F)F] | G2-2 |
| 16.37 | 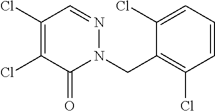 [SMILES: O=c1c(Cl)c(Cl)cnn1Cc1c(Cl)cccc1Cl] | G2-3 |
| 16.39 | 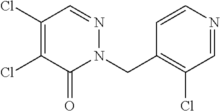 [SMILES: O=c1c(Cl)c(Cl)cnn1Cc1ccncc1Cl] | G3-1 |
| 16.40 | 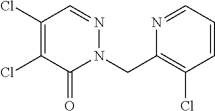 [SMILES: O=c1c(Cl)c(Cl)cnn1Cc1ncccc1Cl] | G3-2 |
| 16.41 | 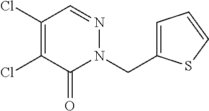 [SMILES: O=c1c(Cl)c(Cl)cnn1Cc1cccs1] | G3-3 |
| 16.42 | 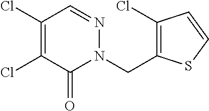 [SMILES: O=c1c(Cl)c(Cl)cnn1Cc1sccc1Cl] | G3-4 |

### General Experimental Methods

1H NMR spectra were recorded on Bruker Avance III 400 MHz and Varian Mercury plus 300 MHz and TMS was used as an internal standard.

LCMS was taken on a quadrupole Mass Spectrometer on Agilent LC/MSD 1200 Series (Column: C18 (50×4.6 mm, 5 μm) operating in ES (+) or (−) ionization mode; T=30 OC; flow rate=1.5 mL/min; detected wavelength: 214 nm.

### Synthesis of 5-chloro-2-(2-chlorobenzyl)pyridazin-3(2H)-one (G1-1)

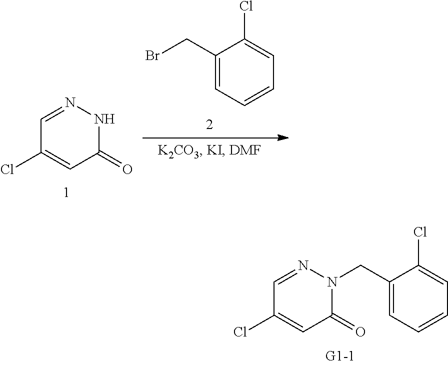 [SMILES: O=c1cc(Cl)cn[nH]1 ; Clc1ccccc1CBr ; O=c1cc(Cl)cnn1Cc1ccccc1Cl]

To a solution of compound 1 (200 mg, 1.53 mmol), compound 2 (378 mg, 1.84 mmol) and K2CO3 (423 mg, 3.06 mmol) in DMF (3 mL) was added KI (25 mg, 0.15 mmol). The solution was stirred at 90° C. for 2 h. The mixture was cooled to room temperature and quenched with water, extracted with EtOAc for 3 times, combined the organic layer, washed with brine, dried over Na2SO4, filtered, concentrated under reduced pressure, purified by column chromatography [eluting with PE to PE/EtOAc (4:1)] to give compound G1-1 (210 mg, 54%) as a yellow solid.

¹H NMR (DMSO-d6, 300 MHz): δ 5.31 (s, 2H), 7.14 (d, J=7.2 Hz, 1H), 7.27-7.37 (m, 3H), 7.49 (d, J=7.8 Hz, 1H), 8.10 (d, J=2.1 Hz, 1H); LCMS [mobile phase: 10-95% Acetonitrile+0.02% NH4OAc] purity is >95%, Rt=4.239 min; MS Calcd.: 254; MS Found: 255 (M+1)⁺.

### Synthesis of 4,5-dibromo-2-(2-chlorobenzyl)pyridazin-3(2H)-one (G1-4)

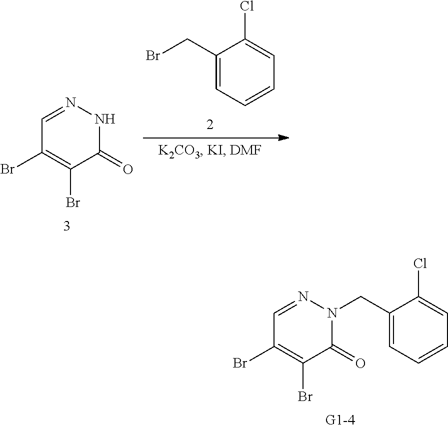 [SMILES: O=c1[nH]ncc(Br)c1Br ; Clc1ccccc1CBr ; O=c1c(Br)c(Br)cnn1Cc1ccccc1Cl]

To a solution of compound 3 (150 mg, 0.59 mmol), compound 2 (121 mg, 0.59 mmol) and K2CO3 (163 mg, 1.18 mmol) in DMF (3 mL) was added KI (10 mg, 0.06 mmol). The solution was stirred at 90° C. for 2 h. The mixture was cooled to room temperature and quenched with water, extracted with EtOAc for 3 times, combined the organic layer, washed with brine, dried over Na2SO4, filtered, concentrated under reduced pressure to give the crude product and washed with CH3OH to give compound G1-4 (80 mg, 36%) as a gray solid.

¹H NMR (DMSO-d₆, 300 MHz): δ 5.36 (s, 2H), 7.19 (d, J=11.1 Hz, 1H), 7.27-7.38 (m, 2H), 7.50 (d, J=8.1 Hz, 1H), 8.20 (s, 1H); LCMS [mobile phase: 30-95% Acetonitrile+0.02% NH₄OAc] purity is >95%, Rt=3.849 min; MS Calcd.: 377; MS Found: 378 (M+1)⁺.

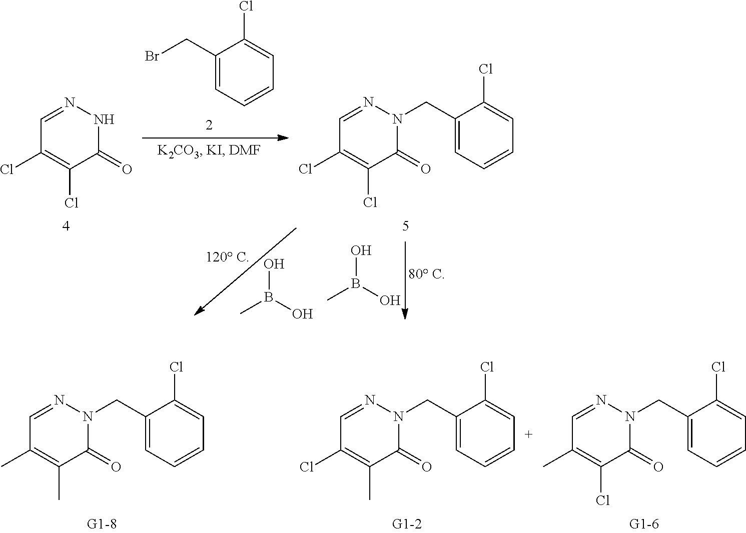

### Synthesis of 4,5-dichloro-2-(2-chlorobenzyl)pyridazin-3(2H)-one (5)

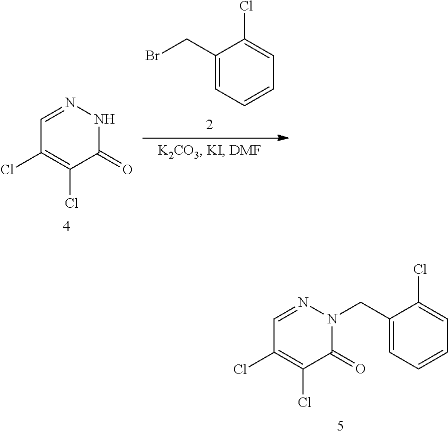 [SMILES: O=c1[nH]ncc(Cl)c1Cl ; Clc1ccccc1CBr ; O=c1c(Cl)c(Cl)cnn1Cc1ccccc1Cl]

To a solution of compound 4 (3 g, 18.2 mmol), compound 2 (4.5 g, 21.8 mmol) and _{K2CO3} (5 g, 36.4 mmol) in DMF (30 mL) was added KI (0.3 g, 1.8 mmol). The solution was stirred at 90° C. for 2 h. The mixture was cooled to room temperature and quenched with water, extracted with EtOAc for 3 times, combined the organic layer, washed with brine, dried over Na₂SO₄, filtered, concentrated under reduced pressure, purified by column chromatography [eluting with PE to PE/EtOAc (10:1)] to give compound 5 (5 g, 96%) as a white solid.

### Synthesis of 5-chloro-2-(2-chlorobenzyl)-4-methylpyridazin-3(2H)-one (G1-2) and 4-chloro-2-(2-chlorobenzyl)-5-methylpyridazin3 (2H)-one (G1-6)

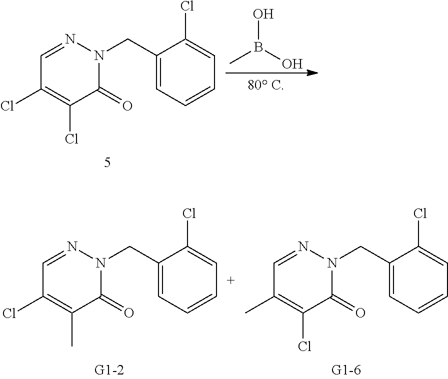

To a solution of compound 5 (900 mg, 3.1 mmol), Methylboronic acid (187 mg, 3.1 mmol), TBAB (100 mg, 0.3 mmol) and _{K2CO3} (1074 mg, 7.8 mmol) in Dioxane/H₂O (10 mL/3 mL) was added Pd(PPh₃)₂Cl₂ (219 mg, 0.3 mmol). The solution was stirred at 80° C. overnight. The mixture was cooled to room temperature and quenched with water, extracted with EtOAc for 3 times, combined the organic layer, washed with brine, dried over Na₂SO₄, filtered, concentrated under reduced pressure, purified by HPLC to give G1-2 (115 mg) and G1-6 (130 mg) as a white solid.

G1-2: ¹H NMR (DMSO-d₆, 400 MHz): δ 2.19 (s, 3H), 5.33 (s, 2H), 7.12 (d, J=7.2 Hz, 1H), 7.287.36 (m, 2H), 7.49 (d, J=7.8 Hz, 1H), 8.07 (s, 1H); LCMS [mobile phase: 30-95% Acetonitrile+0.02% NH₄OAc] purity is >95%, Rt=3.718 min; MS Calcd.: 269; MS Found: 270 (M+1)⁺.

G1-6: ¹H NMR (DMSO-d₆, 400 MHz): δ 2.29 (s, 3H), 5.37 (s, 2H), 7.11 (d, J=7.5 Hz, 1H), 7.297.37 (m, 2H), 7.49 (d, J=7.8 Hz, 1H), 7.98 (s, 1H); LCMS [mobile phase: 30-95% Acetonitrile+0.02% NH₄OAc] purity is >95%, Rt=3.237 min; MS Calcd.: 269; MS Found: 270 (M+1)⁺.

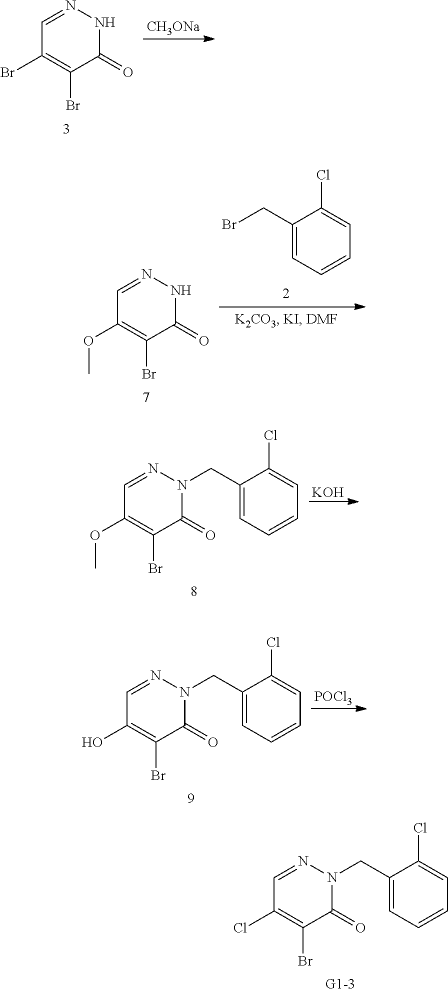

### Synthesis of 4-bromo-5-methoxypyridazin-3(2H)-one (7)

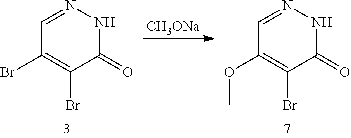

To a solution of compound 3 (4 g, 15.7 mmol) in CH3OH (50 mL) was added CH3ONa (2.6 g, 47.2 mmol). The solution was stirred at 80° C. overnight. The mixture was concentrated under reduced pressure, purified by column chromatography [eluting with PE to PE/DCM/MeOH (10:1:1)] to give compound 7 (1.1 g, 35%) as a white solid.

### Synthesis of 4-bromo-2-(2-chlorobenzyl)-5-methoxypyridazin-3(2H)-one (8)

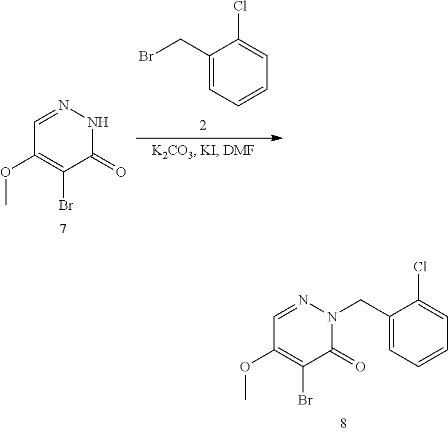

To a solution of compound 7(1 g, 4.88 mmol), compound 2 (1.1 g, 5.37 mmol) and _{K2CO3} (1.3 g, 9.76 mmol) in DMF (15 mL) was added KI (81 mg, 0.49 mmol). The solution was stirred at 90° C. for 2 h. The mixture was cooled to room temperature and quenched with water, extracted with EtOAc for 3 times, combined the organic layer, washed with brine, dried over Na2SO4, filtered, concentrated under reduced pressure, purified by column chromatography [eluting with PE to PE/EtOAc (5:1)] to give compound 8 (350 mg, 22%) as a white solid.

### Synthesis of 4-bromo-2-(2-chlorobenzyl)-5-hydroxypyridazin-3(2H)-one (9)

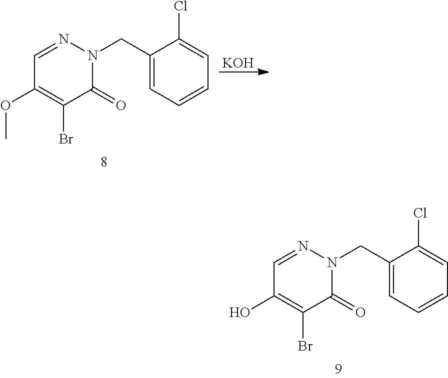

To a solution of compound 8 (350 mg, 1.06 mmol) in H2O (3 mL) was added KOH (119 mg, 2.12 mmol). The solution was stirred at reflux overnight. The mixture was cooled to room temperature and neutralized with concentrated HCl and extracted with EtOAc, washed with brine, dried over Na2SO4, filtered, concentrated under reduced pressure to give compound 9 (322 mg, 91%) as a white solid.

### Synthesis of 4-bromo-5-chloro-2-(2-chlorobenzyl)pyridazin-3(2H)-one (G1-3)

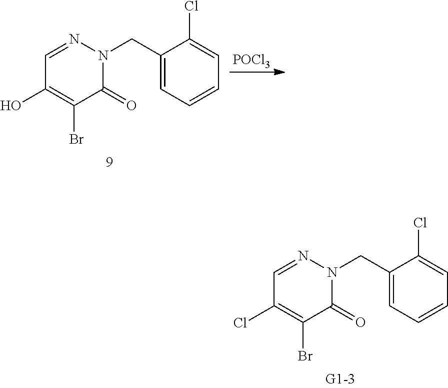

A solution of compound 9 (322 mg, 1.02 mmol) in POCl3 (3 mL) was stirred at 100° C. overnight. The mixture was cooled to room temperature and quenched with water and sat. NaOH, extracted with EtOAc, washed with brine, dried over Na2SO4, filtered, concentrated under reduced pressure, purified by HPLC to give compound G1-3 (122 mg, 36%) as a white solid.

¹H NMR (CDCl3, 300 MHz): δ 5.52 (s, 2H), 7.12-7.28 (m, 3H), 7.38-7.41 (m, 1H), 7.71 (d, J=4.5 Hz, 1H); LCMS [mobile phase: 10-95% Acetonitrile+0.02% NH4OAc] purity is >95%, Rt=4.050 min; MS Calcd.: 333; MS Found: 334 (M+1)⁺.

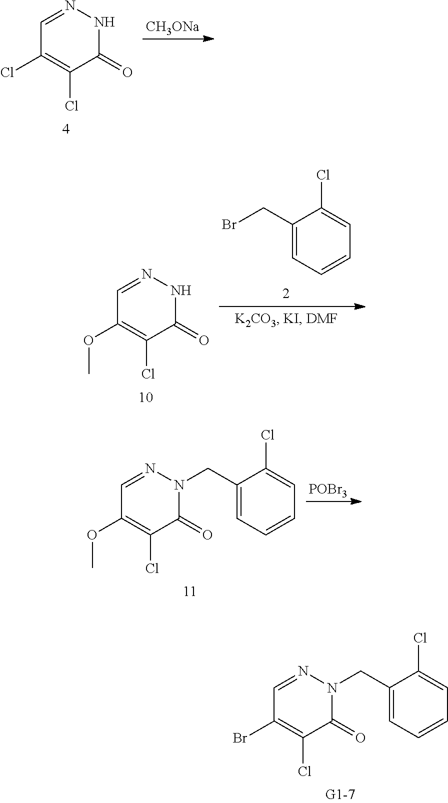

### Synthesis of 4-chloro-5-methoxypyridazin-3(2H)-one (10)

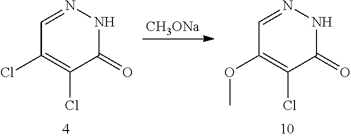

To a solution of compound 4 (4.1 g, 24.8 mmol) in CH₃OH (50 mL) was added CH₃ONa (2.6 g, 74.5 mmol). The solution was stirred at 80° C. overnight. The mixture was concentrated under reduced pressure, purified by column chromatography [eluting with PE to PE/DCM/MeOH (10:1:1)] to give 10 (1.2 g, 30%) as a white solid.

### Synthesis of 4-chloro-2-(2-chlorobenzyl)-5-methoxypyridazin-3(2H)-one (11)

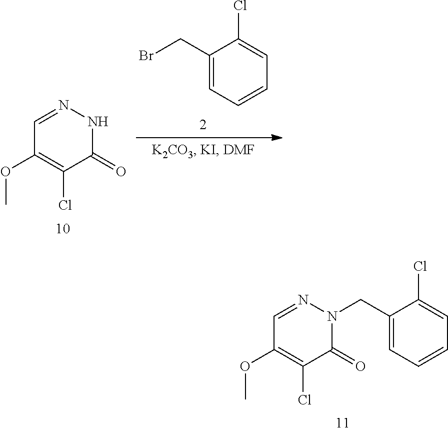

To a solution of compound 10 (1 g, 6.25 mmol), compound 2 (1.5 g, 7.50 mmol) and _{K2CO3} (1.7 g, 12.5 mmol) in DMF (15 mL) was added KI (104 mg, 0.63 mmol). The solution was stirred at 90° C. for 2 h. The mixture was cooled to room temperature and quenched with water, extracted with EtOAc for 3 times, combined the organic layer, washed with brine, dried over Na₂SO₄, filtered, concentrated under reduced pressure, purified by column chromatography [eluting with PE to PE/EtOAc (4:1)] to give compound 11 (700 mg, 44%) as a white solid.

### Synthesis of 5-bromo-4-chloro-2-(2-chlorobenzyl)pyridazin-3(2H)-one (G1-7)

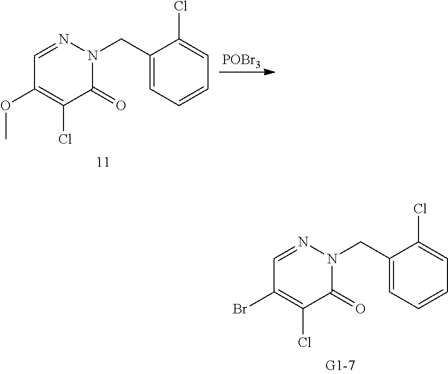

A solution of compound 11 (322 mg, 1.02 mmol) and POBr₃ (4.2 g, 14.7 mmol) was stirred at 100° C. overnight. The mixture was cooled to room temperature and quenched with water and sat. NaOH, extracted with EtOAc, washed with brine, dried over Na₂SO₄, filtered, concentrated under reduced pressure, purified by HPLC to give compound G1-7 (35 mg, 4.3%) as a white solid.

¹H NMR (CDCl₃, 300 MHz): δ 5.47 (s, 2H), 7.23-7.29 (d, J=15.9 Hz, 3H), 7.39-7.41 (d, J=7.2 Hz, 1H), 7.88 (s, 1H); LCMS [mobile phase: 30-95% Acetonitrile+0.02% NH₄OAc] purity is >95%, Rt=4.050 min; MS Calcd.: 333; MS Found: 334 (M+1)⁺.

### Synthesis of 4,5-dichloro-2-(2-(trifluoromethyl)benzyl)pyridazin-3(2H)-one (G2-1)

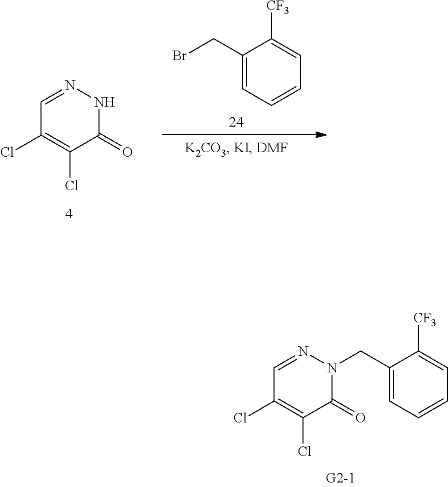 [SMILES: O=c1[nH]ncc(Cl)c1Cl ; FC(F)(F)c1ccccc1CBr ; O=c1c(Cl)c(Cl)cnn1Cc1ccccc1C(F)(F)F]

To a solution of compound 4 (200 mg, 1.21 mmol), compound 24 (348 mg, 1.45 mmol) and K₂CO₃ (335 mg, 2.42 mmol) in DMF (3 mL) was added KI (20 mg, 0.12 mmol). The solution was stirred at 90° C. for 2 h. The mixture was cooled to room temperature and quenched with water, extracted with EtOAc for 3 times, combined the organic layer, washed with brine, dried over Na₂SO₄, filtered, concentrated under reduced pressure, purified by HPLC to give G2-1 (230 mg, 59%) as a white solid.

¹H NMR (DMSO-d₆, 300 MHz): δ 5.46 (s, 2H), 7.23 (d, J=7.5 Hz, 1H), 7.51-7.66 (m, 2H), 7.79 (d, J=7.8 Hz, 1H), 8.29 (s, 1H); LCMS [mobile phase: 30-95% Acetonitrile+0.02% NH₄OAc] purity is >95%, Rt=3.901 min; MS Calcd.: 323; MS Found: 324 (M+1)⁺.

### Synthesis of 4,5-dichloro-2-(2-(trifluoromethoxy)benzyl)pyridazin-3(2)-one (G2-2)

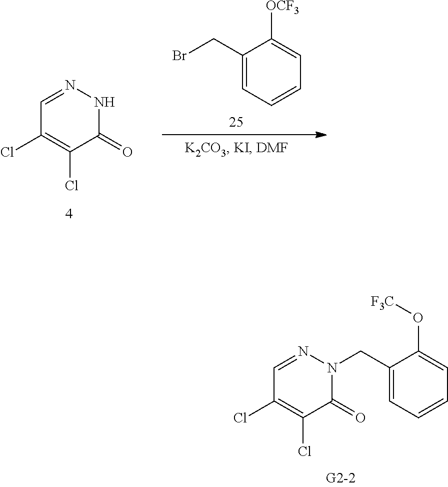 [SMILES: O=c1[nH]ncc(Cl)c1Cl ; FC(F)(F)Oc1ccccc1CBr ; O=c1c(Cl)c(Cl)cnn1Cc1ccccc1OC(F)(F)F]

To a solution of compound 4 (200 mg, 1.21 mmol), compound 25 (371 mg, 1.45 mmol) and K₂CO₃ (335 mg, 2.42 mmol) in DMF (3 mL) was added KI (20 mg, 0.12 mmol). The solution was stirred at 90° C. for 2 h. The mixture was cooled to room temperature and quenched with water, extracted with EtOAc for 3 times, combined the organic layer, washed with brine, dried over Na₂SO₄, filtered, concentrated under reduced pressure, purified by HPLC to give G2-2 (220 mg, 54%) as a white solid.

¹H NMR (DMSO-d₆, 300 MHz): δ 5.36 (s, 2H), 7.36-7.41 (m, 3H), 7.44-7.51 (m, 1H), 8.25 (s, 1H); LCMS [mobile phase: 30-95% Acetonitrile+0.02% NH₄OAc] purity is >95%, Rt=3.989 min; MS Calcd.: 339; MS Found: 340 (M+1)⁺.

### Synthesis of 4,5-dichloro-2-(2,6-dichlorobenzyl)pyridazin-3(2H)-one (G2-3)

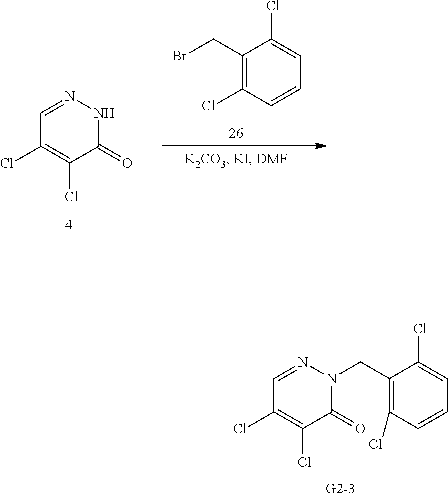 [SMILES: O=c1[nH]ncc(Cl)c1Cl ; Clc1cccc(Cl)c1CBr ; O=c1c(Cl)c(Cl)cnn1Cc1c(Cl)cccc1Cl]

To a solution of compound 4 (200 mg, 1.21 mmol), compound 26 (349 mg, 1.45 mmol) and K₂CO₃ (335 mg, 2.42 mmol) in DMF (3 mL) was added KI (20 mg, 0.12 mmol). The solution was stirred at 90° C. for 2 h. The mixture was cooled to room temperature and quenched with water, extracted with EtOAc for 3 times, combined the organic layer, washed with brine, dried over Na₂SO₄, filtered, concentrated under reduced pressure to give the crude product and washed with CH₃OH to give compound G2-3 (220 mg, 56%) as a brown solid.

¹H NMR (CDCl₃, 300 MHz): δ 5.61 (s, 2H), 7.23-7.28 (m, 1H), 7.35-7.38 (m, 2H), 7.67 (s, 1H); LCMS [mobile phase: 30-95% Acetonitrile+0.02% NH₄OAc] purity is >95%, Rt=3.953 min; MS Calcd.: 324; MS Found: 325 (M+1)⁺.

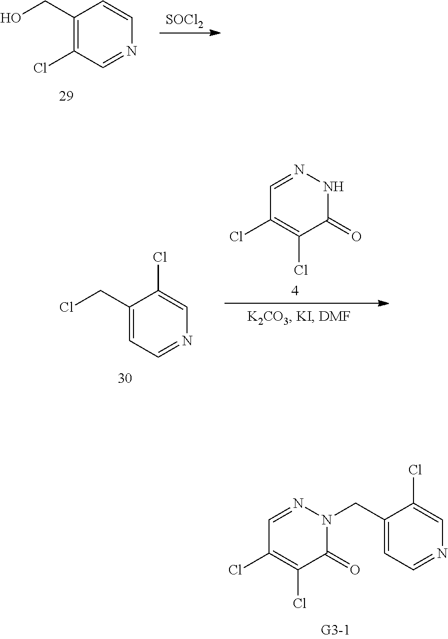

### Synthesis of 3-chloro-4-(chloromethyl)pyridine (30)

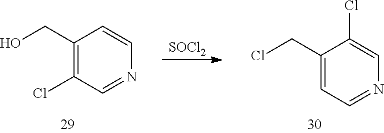

To a solution of compound 29 (250 mg, 1.74 mmol) in DCM (5 mL) was added SOCl₂ (249 mg, 2.09 mmol) and DMF (cat) at 0° C. The mixture was stirred at room temperature for 3 h. Water was added, and the mixture was extracted with DCM, washed with sat. NaHCO₃ and brine, dried over Na₂SO₄, filtered, concentrated under reduced pressure to give compound 30 (240 mg, 85%) as a yellow oil.

### Synthesis of 4,5-dichloro-2-((3-chloropyridin-4-yl)methylpyridazin-3(2H)-one (G3-1)

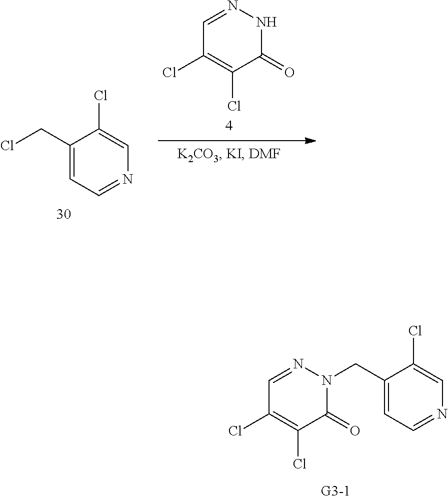 [SMILES: ClCc1ccncc1Cl ; O=c1[nH]ncc(Cl)c1Cl ; O=c1c(Cl)c(Cl)cnn1Cc1ccncc1Cl]

To a solution of compound 4 (200 mg, 1.21 mmol), compound 30 (235 mg, 1.45 mmol) and K2CO3 (335 mg, 2.42 mmol) in DMF (3 mL) was added KI (20 mg, 0.12 mmol). The solution was stirred at 90° C. for 2 h. The mixture was cooled to room temperature and quenched with water, extracted with EtOAc for 3 times, combined the organic layer, washed with brine, dried over Na2SO4, filtered, concentrated under reduced pressure, purified by HPLC to give G3-1 (135 mg, 47%) as a brown solid.

¹H NMR (DMSO-d₆, 300 MHz): δ 5.40 (s, 2H), 7.23 (d, J=5.1 Hz, 1H), 8.31 (s, 1H), 8.47 (d, J=4.8 Hz, 1H), 8.67 (s, 1H); LCMS [mobile phase: 20-95% Acetonitrile+0.02% NH₄OAc] purity is >95%, Rt=3.163 min; MS Calcd.: 291; MS Found: 292 (M+1)⁺.

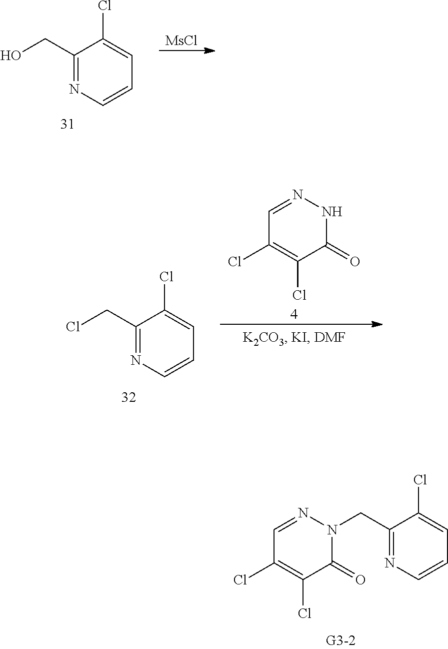

### Synthesis of 3-chloro-2-(chloromethyl)pyridine (32)

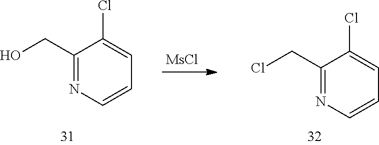 [SMILES: "MsCl" ; OCc1ncccc1Cl ; ClCc1ncccc1Cl]

To a solution of compound 31 (300 mg, 2.1 mmol) in DCM (4 mL) was added DIEA (539 mg, 4.2 mmol) at 0° C., then MsCl (263 mg, 2.3 mmol) was added dropwise at 0° C. The mixture was stirred at room temperature for 31. Water was added, and the mixture was extracted with DCM, washed with sat. NaHCO₃ and brine, dried over Na₂SO₄, filtered, concentrated under reduced pressure to give compound 32 (240 mg, 69%) as a yellow oil.

### Synthesis of 4,5-dichloro-2-((3-chloropyridin-2-yl)methyl)pyridazin-3(2H)-one (G3-2)

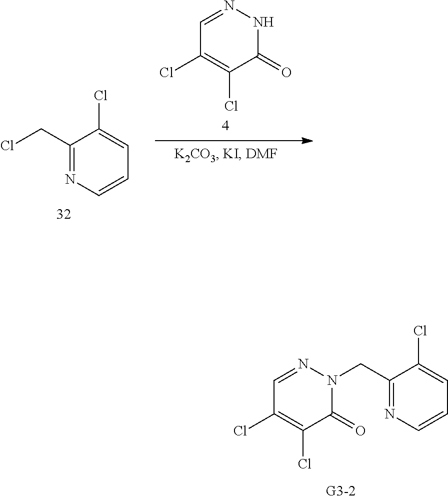 [SMILES: ClCc1ncccc1Cl ; O=c1[nH]ncc(Cl)c1Cl ; O=c1c(Cl)c(Cl)cnn1Cc1ncccc1Cl]

To a solution of compound 4 (200 mg, 1.21 mmol), compound 32 (235 mg, 1.45 mmol) and K₂CO₃ (335 mg, 2.42 mmol) in DMF (3 mL) was added KI (20 mg, 0.12 mmol). The solution was stirred at 90° C. for 2 h. The mixture was cooled to room temperature and quenched with water, extracted with EtOAc for 3 times, combined the organic layer, washed with brine, dried over Na₂SO₄, filtered, concentrated under reduced pressure, purified by HPLC to give G3-2 (126 mg, 42%) as a yellow solid.

¹H NMR (DMSO-d₆, 300 MHz): δ 5.53 (s, 2H), 7.38-7.42 (m, 1H), 7.99 (d, J=8.1 Hz, 1H), 8.27 (s, 1H), 8.41 (d, J=4.5 Hz, 1H); LCMS [mobile phase: 20-95% Acetonitrile+0.02% NH₄OAc] purity is >95%, Rt=3.365 min; MS Calcd.: 290; MS Found: 291 (M+1)⁺.

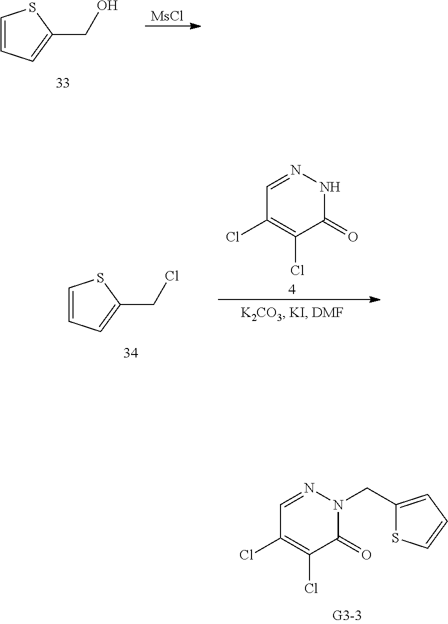

### Synthesis of 2-(chloromethyl)thiophene (34)

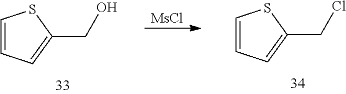 [SMILES: "MsCl" ; OCc1cccs1 ; ClCc1cccs1]

To a solution of compound 33 (300 mg, 2.62 mmol) in DCM (4 mL) was added DIEA (1.02 g, 7.88 mmol) at 0° C., then MsCl (330 mg, 2.89 mmol) was added by dropwise at 0° C. The mixture was stirred at room temperature for 3 h. Water was added, and the mixture was extracted with DCM, washed with sat. NaHCO₃ and brine, dried over Na₂SO₄, filtered, concentrated under reduced pressure to give compound 34 (220 mg, 63%) as a colorless oil.

### Synthesis of 4,5-dichloro-2-(thiophen-2-ylmethyl)pyridazin-3(2H)-one (G3-3)

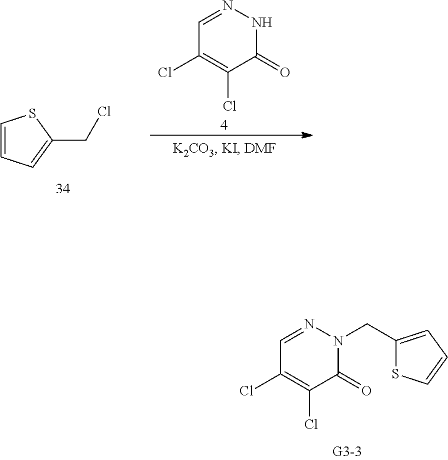

To a solution of compound 4 (200 mg, 1.21 mmol), compound 34 (193 mg, 1.45 mmol) and K₂CO₃ (335 mg, 2.42 mmol) in DMF (3 mL) was added KI (20 mg, 0.12 mmol).

The solution was stirred at 90° C. for 2 h. The mixture was cooled to room temperature and quenched with water, extracted with EtOAc for 3 times, combined the organic layer, washed with brine, dried over Na₂SO₄, filtered, concentrated under reduced pressure, purified by HPLC to give G3-3 (210 mg, 66%) as a yellow solid.

¹H NMR (DMSO-d₆, 300 MHz): δ 5.44 (s, 2H), 6.98-7.00 (m, 1H), 7.16-7.17 (m, 1H), 7.50 (d, J=11.7 Hz, 1H), 8.26 (s, 1H); LCMS [mobile phase: 10-95% Acetonitrile+0.02% NH₄Ac] purity is >95%, Rt=4.210 min; MS Calcd.: 261; MS Found: 262 (M+1)⁺.

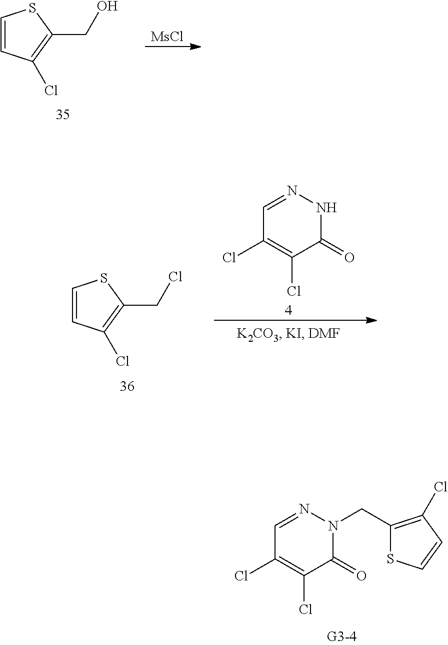

### Synthesis of 3-chloro-2-(chloromethyl)thiophene (36)

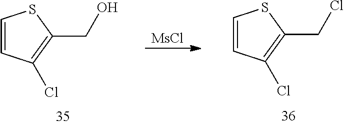 [SMILES: "MsCl" ; OCc1sccc1Cl ; ClCc1sccc1Cl]

To a solution of compound 35 (300 mg, 2.0 mmol) in DCM (4 mL) was added DIEA (521 mg, 4.0 mmol) at 0° C., then MsCl (254 mg, 2.2 mmol) was added by dropwise at 0° C. The mixture was stirred at room temperature for 3 h. Water was added, and the mixture was extracted with DCM, washed with sat. NaHCO₃ and brine, dried over Na₂SO₄, filtered, concentrated under reduced pressure to give compound 36 (250 mg, 74%) as a yellow oil

### Synthesis of 4,5-dichloro-2-((3-chlorothiophen-2-yl)methyl)pyridazin-3(2H)-one (G3-4)

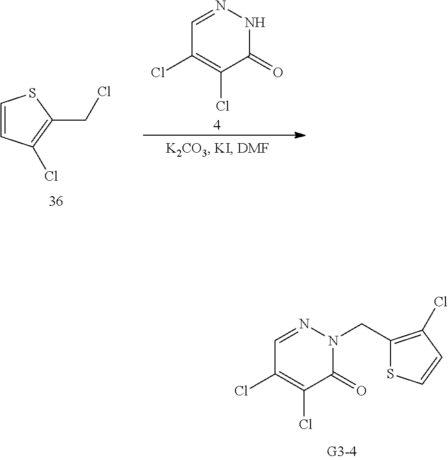

To a solution of compound 4 (200 mg, 1.21 mmol), compound 36 (243 mg, 1.45 mmol) and K₂CO₃ (335 mg, 2.42 mmol) in DMF (3 mL) was added KI (20 mg, 0.12 mmol). The solution was stirred at 90° C. for 2 h. The mixture was cooled to room temperature and quenched with water, extracted with EtOAc for 3 times, combined the organic layer, washed with brine, dried over Na₂SO₄, filtered, concentrated under reduced pressure, purified by HPLC to give G3-4 (125 mg, 41%) as a white solid.

¹H NMR (DMSO-d₆, 300 MHz): δ 5.43 (s, 2H), 7.07 (d, J=5.7 Hz, 1H), 7.06-7.08 (d, J=5.7 Hz, 1H), 7.66-7.68 (d, J=5.4 Hz, 1H), 8.26 (s, 1H); LCMS [mobile phase: 30-95% Acetonitrile+0.02% NH₄OAc] purity is >95%, Rt=3.608 min; MS Calcd.: 296; MS Found: 297 (M+1)⁺.

1. A composition comprising:
a) a compound of Formula I: 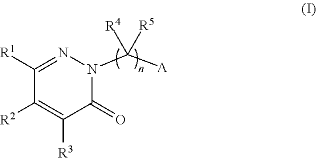
or a pharmaceutically acceptable salt thereof, wherein:
R¹, R² and R³ are independently selected from the group consisting of hydrogen, deuterium, halogen, cyano, optionally substituted alkyl, optionally substituted cyclyl, optionally substituted heterocyclyl, optionally substituted aryl, optionally substituted heteroaryl, OR⁶, CO₂R⁶, C(O)NR⁶R⁷, OC(O)R⁶, N(R⁶)C(O)R⁶, NR⁶R⁷, SR⁶, S(O)—R⁶, SO₂R⁶, OS(O)₂R⁶, SO₂NR⁶NR⁷, and NO₂;
R⁴ and R⁵ are independently hydrogen, deuterium, optionally substituted alkyl, or halogen, or R⁴ and R⁵ together with the carbon they are attached to form an optionally substituted cyclic alkyl or optionally substituted heterocyclic;
R⁶ and R⁷ are independently for each occurrence hydrogen, optionally substituted alkyl, optionally substituted cyclyl, optionally substituted heterocyclyl, optionally substituted aryl, or optionally substituted heteroaryl;
A is aryl, heteroaryl, cyclyl, heterocyclyl, or alkyl, each of which can be optionally substituted; and
n is 0, 1, or 2; and
b) a pharmaceutically acceptable carrier.

2. The composition of claim 1, wherein R¹ is hydrogen.

3. The composition of claim 1, wherein R² is a halogen, NO₂, OS(O)₂R⁶, cyano, hydroxyl, alkoxy, or alkylthio.

4. The composition of claim 1, wherein R³ is halogen, heterocyclyl, alkoxy, or alkylamino.

5. The composition of claim 1, wherein R² is a halogen; hydroxyl, alkoxy, or alkylthio; and R³ is a halogen; heterocyclyl; hydroxyl, alkoxy, or alkylthio.

6. The composition of claim 5, wherein R² is Cl, Br, I, F, NO₂, OH, methoxy (—OCH₃), ethoxy (—OEt), mesylate (—OS(O)₂Me), triflate (—OS(O)₂CF₃), besylate (—OS(O)₂Ph), tosylate (—OS(O)₂C6H₄CH₃), methylthio (—SCH₃), or ethylthio (—SCH₂CH₃).

7. The composition of claim 1, wherein R³ is Cl, Br, optionally pyrrolidinyl, methoxy, ethoxy (—OCH₂CH₃) or butylamino (—NH(CH₂)₃CH₃).

8. The composition of claim 7, wherein R² is Cl, and R³ is Cl, methoxy, ethoxy, pyrrolidinyl, or butylamino; R² is hydroxyl, methoxy, or ethylthio, and R³ is Cl; R² and R³ are both Br; or R² and R³ are both methylthio.

9. The composition of claim 1, wherein R¹ is hydrogen, and R² is Cl, and R³ is Cl, methoxy, ethoxy, pyrrolidinyl, or butylamino; R¹ is hydrogen, and R² is hydroxyl, methoxy, or ethylthio, and R³ is Cl; R¹ is hydrogen, and R² and R³ are both Br; or R¹ is hydrogen, and R² and R³ are both methylthio

10. The composition of claim 1, wherein R⁴ and R⁵ are both hydrogen.

11. The composition of claim 1, wherein n is 0 or 1.

12. The composition of claim 1, wherein A is an optionally substituted C₁-C₆alkyl, optionally substituted aryl or optionally substituted heteroaryl.

13. The composition of claim 12, wherein A is an optionally substituted aryl of structure 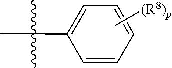
wherein R⁸ is independently for each occurrence deuterium, halogen, cyano, optionally substituted alkyl, optionally substituted cyclyl, optionally substituted heterocyclyl, optionally substituted aryl, optionally substituted heteroaryl, OR⁹, C(O)OR⁹, C(O)NR⁹R¹⁰, OC(O)R⁹, N(R⁹)C(O)R⁹, NR⁹R¹⁰, SR⁹, S(O)R⁹, SO₂R⁹, SO₂NR⁹NR¹⁰, and NO_{2;} and p is 0, 1, 2, 3, 4, or 5, wherein R⁹ and R¹⁰ are independently for each occurrence hydrogen, optionally substituted alkyl, optionally substituted cyclyl, optionally substituted heterocyclyl, optionally substituted aryl, or optionally substituted heteroaryl.

14. The composition of claim 13, wherein p is 0, 1, 2, or 3.

15. The composition of claim 14, wherein R⁸ is halogen, C₁-C₆alkyl, NO₂, hydroxyl, alkoxy, alkylthio, CF₃, OCF₃, C(O)OR⁹, C(O)NR⁹R¹⁰, or CN.

16. The composition of any claim 15, wherein optionally substituted aryl is phenyl; 2-substituted phenyl; 3-substituted phenyl; 2,6-disubstituted phenyl, wherein substituents at the 2-position and 6-position are independently selected; 4-substituted phenyl;’ or 2,3,6-trisubstituted phenyl, wherein substituents at the 2-, 3-, and 6-positions are independently selected.

17. The composition of claim 12, wherein A is an optionally substituted naphthalene.

18. The composition of claim 17, wherein the optionally substituted naphthalene is 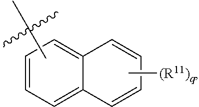
wherein R¹¹ independently for each occurrence deuterium, halogen, cyano, optionally substituted alkyl, optionally substituted cyclyl, optionally substituted heterocyclyl, optionally substituted aryl, optionally substituted heteroaryl, OR¹², C(O)OR¹³, C(O)NR¹²R¹³, OC(O)R¹², N(R¹²)C(O)R¹², NR¹²R¹³, SR¹², S(O)R¹², SO₂R¹², SO₂NR¹²NR¹³, and NO_{2;} and q is 0, 1, 2, 3, 4, 5, 6, or 7, wherein R¹² and R¹³ are independently for each occurrence are independently for each occurrence hydrogen, optionally substituted alkyl, optionally substituted cyclyl, optionally substituted heterocyclyl, optionally substituted aryl, or optionally substituted heteroaryl.

19. The composition of claim 18, wherein the optionally substituted naphthalene is

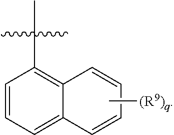

20. The composition of claim 19, wherein q is 0 or 1.

21. The composition of claim 12, wherein A is an optionally substituted heteroaryl containing 1-2 sulfur, 1-4 nitrogen, or 1-2 oxygen atoms.

22. The composition of claim 21, wherein the optionally substituted heteroaryl is an optionally substituted thiophene, optionally substituted pyridine or optionally substituted pyrimidine.

23. The composition of claim 1, wherein A is selected from the group consisting of methyl, phenyl; 2-bromophenyl; 2-fluorophenyl; 2-chlorophenyl; 2-methylphenyl; 3-methylphenyl; 2-nitrophenyl; 2-cyanophenyl; 2-chloro-6-fluorophenyl; 4-nitrophenyl; 4-chlorophenyl; 4-bromophenyl; 3-methoxyphenyl; 3-cyanophenyl; 2,3,6-trichlorophenyl; 4-aminoformylphenyl; 4-methoxycarbonylphenyl; thiophen-2-yl; 3-chlorothiophen-2-yl; pyridin-2-yl; 3-chloropyridin-2-yl, pyridine-4-yl; 3-chloropyridin-4-yl; naphthalen-1-yl; or 4,6-dimethylpyrimidin-2-yl.

24. The composition of claim 1, wherein the compound of Formula I is a compound from Table 1.

25. The composition of claim 1 further comprising an antibiotic.

26-50. (canceled)

51. A matrix impregnated with a composition of claim 1.

52. The matrix of claim 51 that is a gel coating specifically formulated for slow release of the antibacterial composition into a surrounding aqueous environment.

53. A method comprising administering a therapeutically effective amount of a pharmaceutical composition of claim 1 to a subject with a bacterial infection.

54-56. (canceled)

57. A method of inhibiting the development of resistance to an antibiotic by a bacteria comprising, contacting the bacteria with an effective amount of a composition of claim 1 and with an effective amount of the antibiotic.

58-69. (canceled)
